(19) United States
(12) Patent Application Publication
(10) Pub. No.: US 2023/0190613 A1
(43) Pub. Date: Jun. 22, 2023

(54) SKIN PERFECTING COSMETIC COMPOSITIONS AND METHODS OF USE

(71) Applicant: L'OREAL, Paris (FR)

(72) Inventors: Prabhjot K. SAINI, Avenel, NJ (US); Anne-Laure BERNARD, New York, NY (US); Yixin YANG, Union, NJ (US)

(73) Assignee: L'OREAL, Paris (FR)

(21) Appl. No.: 17/967,305

(22) Filed: Oct. 17, 2022

Related U.S. Application Data
(60) Provisional application No. 63/287,650, filed on Dec. 9, 2021.

(30) Foreign Application Priority Data
Jan. 31, 2022 (FR) 22 00807

(51) Int. Cl. A61K 8/58; A61K 8/34; A61K 8/31; A61K 8/06; A61Q 19/00; A61K 8/81; A61K 8/891; A61K 8/02

(52) U.S. Cl. CPC A61K 8/585; A61K 8/31; A61K 8/062; A61K 8/0241; A61K 8/345; A61K 8/891; A61K 8/8152; A61Q 19/00; A61K 2800/10; A61K 2800/43; A61K 2800/262

(57) ABSTRACT

Skin tightening compositions for application to the skin for providing an instantaneous and dramatic improvement to the appearance of skin. The skin tightening compositions typically include a dispersion (A) comprising i) at least two particle consisting of an ethylenic polymer, ii) at least stabilizers comprising at least one (C3-C12)cycloalkyl group, iii) at least one hydrocarbon-based fatty substance which is liquid at 20° C. and 1 atmosphere, iv) at least one polyol which is liquid at 20° C. and 1 atmosphere and optionally v) water, one ore more polyols, in addition to the one or more polyol(s) from A)iv), which may be the same or different than the one from A)iv); one or more volatile hydrocarbon, in addition to the one or more volatile hydrocarbon(s) from A)iii), which may be the same or different than the one from A)iii); one or more fillers.

### CROSS-REFERENCE TO RELATED APPLICATIONS

This application claims priority to U.S. Provisional Pat. Application No. 63/287,650 filed on Dec. 09, 2021, and French Application No. 22 00807 filed on Jan. 31, 2022, both of which are titled SKIN PERFECTING COSMETIC COMPOSITIONS AND METHODS OF USE, the entirety of which are incorporated herein by reference.

### FIELD OF THE DISCLOSURE

The instant disclosure relates to skin tightening compositions for application to the skin for providing an instantaneous and dramatic improvement to the appearance of skin, for example, by reducing the appearance of wrinkles, eye bags, pores, and skin imperfections such as scarring, dark spots (and uneven skin tone), dark circles, and roughness.

### BACKGROUND

Skin produces less collagen and elastin as it ages. For example, after the age of twenty, a person (human) produces about 1 percent less collagen in the skin each year. As a result, the skin becomes thinner and more fragile. Inevitably, wrinkles, crow’s feet, age-spots, eye bags, and the like, begin to form.

Consumers often wish to improve the appearance of such age-related skin imperfections, preferably with instantaneous results. Many consumer products and procedures devoted to hiding and reducing wrinkles are available. Some products and procedures are simple and inexpensive, for example, applying make-up, particularly a primer or colored foundation, to cover the skin (and thereby cover and/or fill the wrinkles and provide a smoother look). Far more expensive and drastic procedures, such as surgical face lifts and Botox® injections, are also used to reduce the appearance of wrinkles. However, many consumers either cannot afford, or do not wish, to undergo such drastic cosmetic procedures. There are a number of lotions and creams which are formulated to hydrate the skin and make it more supple, thereby reducing the appearance of wrinkles. Some of these products contain active ingredients, for example, niacinamide, that help repair and rejuvenate skin over time. Unfortunately, however, all of these products and procedures have drawbacks.

Make-up products are often visible, offer minimal texture benefits, and have no long-term lasting effect on the skin. After removal of the make-up, the skin looks the same as before the make-up was applied. Common skin care products can have chronic, acute or both effects on the skin. Hydration and optical effects are common acute benefits, but these benefits quickly wear-off over time.

Attempts have been made to develop new categories of products to improve the appearance of skin without the drawbacks of existing products and procedures. One such family of products can be generally classified as “adhesive, contractile film formers”. Film formers are chemical compositions that when applied to skin, leave a pliable, cohesive and continuous covering. A select group of film formers are also adhesive to the skin and contractile.

Cosmetic products often require the use of a film-forming polymer to obtain a deposit of the product on keratin materials that has good cosmetic properties. In particular, it is necessary for the film-forming deposit to have good persistence, in particular for the deposit not to transfer during contact with the fingers or clothing, and also good resistance on contact with water, notably rain or during showering and for the deposit to be insensitive to perspiration or sebum, and also food fats, notably dietary fats such as oils.

It is known practice to use dispersions of polymer particles, in organic media such as hydrocarbon-based oils. Polymers are notably used as film-forming agents in makeup products such as mascaras, eyeliners, eyeshadows or lipsticks. EP 0 749 747 describes in the examples dispersions in hydrocarbon-based oils (liquid paraffin, isododecane) of acrylic polymers stabilized with polystyrene/copoly(ethylene-propylene) diblock copolymers. The film obtained after application of the dispersion to the skin is sparingly glossy. FR 1 362 795 also describes the use of dispersions of surface-stabilized polymer particles containing hydrocarbon-based oils for making up the lips and the eyelashes. WO 2010/046 229 describes dispersions in isododecane of stabilized acrylic polymers. FR 1 362 795 describes the use of dispersions of surface-stabilized polymer particles containing hydrocarbon-based oils for making up the lips and the eyelashes.

### SUMMARY OF THE DISCLOSURE

The instant disclosure relates to skin tightening compositions comprising a dispersion (A) comprising at least two particles i) consisting of a polymer of ethylenic monomers, ii) at least one stabilizing agent comprising at least one (C₃-C₁₂) cycloalkyl group , iii) at least one hydrocarbon-based fatty substance which is liquid at 20° C. and 1 atmosphere, iv) at least one polyol which is liquid at 20° C. and 1 atmosphere and optionally v) water, one ore more polyols, in addition to the one or more polyol(s) from A)iv), which may be the same or different than the one from A)iv); one or more volatile hydrocarbon, in addition to the one or more volatile hydrocarbon(s) from A)iii), which may be the same or different than the one from A)iii); one or more fillers; and wherein the weight ratio of the total amount of i) of the dispersion A to the total amount of the amount of polyols B is between around 1:1 to 4:1; wherein the weight ratio of the total amount of i) of the dispersion A to the total amount of filler D (dispersion A: filler) is between around 1:1 to 10:1; wherein all percentage by weight are based on the total weight of the skin tightening composition.

The instant disclosure also relates to a process for treating keratin materials, notably human keratin materials such as the skin, the eyelashes or the eyebrows, involving the application to said materials of at least one dispersion (A); to a process for preparing the dispersion (A), to the process for preparing the polymer material (B) obtained from the dispersion (A), to the polymer material (C).

Users are in search of water-based presentation forms, containing as few volatile compounds as possible. Environmental challenges are also tending towards minimizing the use of volatile compounds. Thus, one of the aims of the instant disclosure is to provide a composition with a reduced content of volatile compounds. Another aim is to provide a film-forming composition containing an aqueous phase, affording access to a wider formulation range. It is also sought to include as few surfactants as possible, or even not to include any, in the cosmetic composition for environmental reasons, but also to avoid potential long-term impairment of the keratin materials. Cosmetic compositions containing few or no surfactants in products intended to be applied to keratin materials are thus increasingly sought by users.

Also, it would be advantageous to provide an emulsifying composition, which is capable of forming a stable emulsion in the presence of water, of hydrogel type with a very small amount (<5% by weight relative to the total weight of the composition) or even in the absence of surfactant, notably nonionic and/or ethoxylated surfactant which are of petrochemical origin.

A hydrogel is a gel in which the swelling agent is usually water. The matrix of a hydrogel is generally a network of water-insoluble polymers which are capable of swelling substantially in the presence of a large amount of water or of aqueous composition. Hydrogels based on polymers of poly(acrylic acid) polymer type are used, for example, in beauty masks for the purpose of moisturizing the skin, restoring its elasticity or as anti-ageing agents (Journal of Materials Science: Materials in Medicine, S. Mitura et al. 31:50, pages 1-14 (2020), https://doi.org/10.1007/s10856-020-06390-w). For beauty masks, use is notably made of tacky hydrogels in the presence of crosslinking agents (see, for example, WO 2014/077 519 A1 and FR 2 924 615). Moreover, acrylate-based hydrogels are widely applied in hygiene products for absorbing fluids as they can do so to keep moisture far from the skin, promoting healthy skin, preventing nappy/buttock rash and affording comfort (Journal of Materials Science: Materials in Medicine, S. Mitura et al. 31:50 (2020)).

Another aim of the instant disclosure is to provide a skin tightening composition for treating keratin materials, in particular the skin, preferably human skin and more preferentially facial skin, which is adhesive and if possible not tacky, which has good persistence with respect to external attacking factors, and over time, does not leach, and is resistant to sweat and sebum and insensitive to oils such as dietary oils. Furthermore, the dispersion may comprise cosmetic active agents such as those for obtaining a skin-tensioning effect, for caring for the body, the face and the hair, for protecting against ultraviolet (UV), or for making up the face, the lips, the eyelashes, the eyebrows and the hair. Said composition may be intended in particular for care and / or make-up, in particular for the face and / or the neck.

It is also sought to obtain polymer materials which, when applied to the substrate, have elastic mechanical strength properties, are flexible and can espouse the surface, or even fill the surface irregularities or imperfections of the substrate such as wrinkles, fine lines, comedones, blackheads, scars, etc. It would also be advantageous for said materials to have sufficient affinity for the substrate so that they do not readily detach from the substrate but so that they can, nevertheless, be removed if a sufficient force is exerted by hand, for example with a nail, without this being painful. It is very difficult to find materials which offer this good compromise of substrate adhesion/removal from the support, notably when the support is flexible and when it bears imperfections, such as the skin.

In order to address the above technical problems, the instant disclosure describes skin tightening compositions that have a unique composition, which surprisingly do not compromise the performance of the resultant film, including but not limited to mechanical strength, elasticity, appearance when compared to un-tinted formula.

Skin tightening compositions according to an aspect of the instant disclosure typically include:
A. a dispersion (A), which comprises:
   i) at least two particle (s) of different chemical structures consisting of polymer (s) obtained by polymerization of monomers chosen from ethylenic monomers of:
      a₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino,
      a₂) poly[oxy(C₁-C₄)alkylene] (C₁-C₄)(alkyl)acrylate,
      a₃) ethylenic monomers comprising one or more groups selected from carboxy, anhydride, phosphoric acid, and sulfonic acid;
      it being understood that each particle results from the polymerization of one or more ethylenic monomer(s) a3); and
   ii) one or more polymeric stabilizing agent (s) chosen from:
      d) ethylenic polymers of (C1-C6) (alkyl) acrylate of (C3-C12) cycloalkyl monomers, preferably ethylenic homopolymers of (C1-C6) (alkyl) acrylate of (C3-C12) cycloalkyl, more preferably ethylenic homopolymers of (C3-C12) cycloalkyl (meth) acrylate; and
      e) copolymers of ethylenic monomers of:
         e1) (C1-C6) (C3-C12) cycloalkyl (alkyl) acrylate, and
         e2) (C₁-C₄)alkyl (C₁-C₄)(alkyl)acrylate, preferably copolymers of (C3-C12) cycloalkyl (meth) acrylate and (C1- C4) alkyl (meth) acrylate; and
   iii) one or more liquid fatty substances at 20° C. and 1 atmosphere, preferably volatile hydrocarbon (s); and iv) one or more polyol (s) liquid (s) at 20° C. and 1 atmosphere; and v) possibly water; and
   vi) optionally one or more cosmetic active (s) other than iii) and iv), chosen from f) dyes, g) pigments; h) active agents for caring for keratin materials, in particular the skin, in particular antioxidants and moisturizers other than iv), i) UV filters (A) and / or (B), or j) mixtures of f) to i).
B. one ore more polyols, in addition to the one or more polyol(s) from A)iv), which may be the same or different than the one from A)iv);
C. one or more volatile hydrocarbon, in addition to the one or more volatile hydrocarbon(s) from A)iii), which may be the same or different than the one from A)iii);
D. one or more fillers; and
wherein the weight ratio of the total amount of i) of the dispersion A to the total amount of the amount of polyols B is between around 1:1 to 4:1;
wherein the weight ratio of the total amount of i) of the dispersion A to the total amount of filler D (dispersion A: filler) is between around 1:1 to 10:1;
wherein all percentages by weight are based on the total weight of the skin tightening composition.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

It is to be understood that both the foregoing general description and the following detailed description are exemplary and explanatory only and are not restrictive of the disclosure.

### DETAILED DESCRIPTION OF THE DISCLOSURE

The instant disclosure is directed to skin tightening compositions for providing an instantaneous and dramatic improvement to the appearance of skin by instantly tighten, and/or compress, and/or blur and provide optimal color coverage to facial skin imperfections such as fine lines and deep wrinkles, eye bags, pores, depressed scars such as acne scars, pimples, dark circles and other skin color imperfections, with good wear and comfort throughout the day.

Suitable components, such as those listed below, may be included or excluded from the formulations for the skin-tightening compositions depending on the specific combination of other components, the form of the skin-tightening compositions, and/or the use of the formulation.

These technical problems have been solved by the use of skin tightening compositions comprising a dispersion (A) in particular in hydrogel form for treating keratin materials, in particular α) keratin fibres, notably human keratin fibres such as the eyelashes and the eyebrows or β) human skin, in particular of the face, in which the dispersion (A) comprises:
A. a dispersion (A), which comprises:
   i) at least two particle (s) of different chemical structures consisting of polymer (s) obtained by polymerization of monomers chosen from ethylenic monomers of:
      a₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino,
      a₂) poly[oxy(C₁-C₄)alkylene] (C₁-C₄)(alkyl)acrylate,
      a₃) ethylenic monomers comprising one or more groups selected from carboxy, anhydride, phosphoric acid, and sulfonic acid;
      it being understood that each particle results from the polymerization of one or more ethylenic monomer(s) a3); and
   ii) one or more polymeric stabilizing agent (s) chosen from:
      d) ethylenic polymers of (C1-C6) (alkyl) acrylate of (C3-C12) cycloalkyl monomers, preferably ethylenic homopolymers of (C1-C6) (alkyl) acrylate of (C3-C12) cycloalkyl, more preferably ethylenic homopolymers of (C3-C12) cycloalkyl (meth) acrylate; and
      e) copolymers of ethylenic monomers of:
         e1) (C1-C6) (C3-C12) cycloalkyl (alkyl) acrylate, and
         e2) (C₁-C₄)alkyl (C₁-C₄)(alkyl)acrylate, preferably copolymers of (C3-C12) cycloalkyl (meth) acrylate and (C1- C4) alkyl (meth) acrylate; and
   iii) one or more liquid fatty substances at 20° C. and 1 atmosphere, preferably volatile hydrocarbon (s); and iv) one or more polyol (s) liquid (s) at 20° C. and 1 atmosphere; and v) possibly water; and
   vi) optionally one or more cosmetic active (s) other than iii) and iv), chosen from f) dyes, g) pigments; h) active agents for caring for keratin materials, in particular the skin, in particular antioxidants and moisturizers other than iv), i) UV filters (A) and / or (B), or j) mixtures of f) to i).
B. one ore more polyols, in addition to the one or more polyol(s) from A)iv), which may be the same or different than the one from A)iv);
C. one or more volatile hydrocarbon, in addition to the one or more volatile hydrocarbon(s) from A)iii), which may be the same or different than the one from A)iii);
D. one or more fillers; and
wherein the weight ratio of the total amount of i) of the dispersion A to the total amount of the amount of polyols B is between around 1:1 to 4:1;
wherein the weight ratio of the total amount of i) of the dispersion A to the total amount of filler D (dispersion A: filler) is between around 1:1 to 10:1;
wherein all percentages by weight are based on the total weight of the skin tightening composition.

More particularly, the subject of the instant disclosure relates to the use of the skin tightening composition comprising the dispersion (A) as defined previously for treating keratin materials, in particular α) keratin fibres, notably human keratin fibres such as the hair, the eyelashes and the eyebrows, and/or for shaping keratin fibres such as the hair, or for treating β) human skin, in particular of the face, preferably for filling skin imperfections such as wrinkles and fine lines or scars while at the same time making up said keratin materials, or for filling skin imperfections such as wrinkles and fine lines or scars while at the same time caring for said keratin materials such as moisturizing the skin. The dispersions (A) of the skin tightening compositions, notably the hydrogels, are stable. Furthermore, after application of the skin tightening compositions comprising the dispersions (A) to the keratin materials, the material obtained is film-forming, adheres to said materials and has a very significant volumizing effect on the keratin materials once water is added. The water may originate from the addition or else from the support, keratin materials, which is wet. Furthermore, the active agents such as pigments are not impaired in terms of colours during the filling of imperfections, wrinkles, fine lines or scars.

Thus, the skin tightening compositions according to the instant disclosure is applied to the skin for the purpose of obtaining a thightening effect on the imperfections of the keratin materials, notably facial skin, the residual polymer material (C) of which after application to said materials and evaporation of the solvents is film-forming and adheres to said keratin materials. Mention may notably be made of applications in the field of makeup (lipsticks, mascaras, filling of wrinkles and fine lines, and hiding of imperfections).

Thus the sking tightening composition comprising the dispersion (A) according to the invention is applied to the skin with the aim of obtaining an effect of filling the imperfections of keratin materials in particular of the skin of the face, and the resulting polymeric material (C) of the application of the dispersion (A) after application to said materials and after evaporation of the solvents of the fatty substance (s) iii) and of the water v) if present, is film-forming and adhesive to said keratin materials. Mention may in particular be made of applications in the field of make-up (lipstick, mascaras, filling in wrinkles, fine lines, and masking of imperfections).

The dispersion (A), and the process for treating keratin materials as defined above make it possible to obtain a treatment of said materials resistant in particular to shampoos, sebum, sweat, and / or water but also to fatty substances, in particular food such as oils. In addition, the dispersion is easy to use in compositions, in particular cosmetic compositions, is easy to manufacture and remains stable over time. In fact, the dispersion (A) in accordance with the present invention makes it possible to obtain deposits of polymeric materials (C) on said keratin materials (substrate) which are very resistant to external attacks, in particular to sebum and to fatty substances which are found in food, in particular liquid fatty substances such as vegetable oils and in particular olive oil. It appears that makeup produced with at least one dispersion (A), in particular lip makeup, is particularly resistant to external attacks such as liquid fatty substances, in particular vs. vegetable oils like olive oil. In addition, the make-up obtained with the dispersions (A) are very aesthetic. These dispersions of polymer particles are found at a high solids content in the liquid hydrocarbon fatty substance (s) iii) which makes them easy to formulate.

Furthermore, the polymeric materials (C) of the instant disclosure obtained after application to the substrate and evaporation of the solvents [water v) and / or fatty substances iii)] of the dispersion (A) are flexible and match the surface, significantly reduce, or even fill in the irregularities and surface imperfections of said particularly flexible substrate, in particular the skin such as the filling of wrinkles, fine lines, scars, and other skin imperfections. The polymeric materials of the invention have a good affinity with the substrate, do not come off the substrate easily but enough so that they can be removed if necessary.

A subject of the instant disclosure is also a process for preparing the dispersion (A) as defined previously, and after evaporation of the water v) if present in the medium and of the fatty substances iii), the polymer material (C) obtained via said process.

A subject of the instant disclosure is also a composition, preferably a cosmetic composition, comprising the dispersion (A) as defined previously, and the process for applying said composition to keratin materials.

A subject of the instant disclosure is also a process for treating keratin materials, preferably α) keratin fibres, notably human keratin fibres such as the hair, the eyelashes or the eyebrows, or β) human skin, in particular of the face and notably of the lips, comprising the application to said materials of at least one dispersion (A) as defined previously.

A subject of the instant disclosure is also the use of a dispersion (A) as defined previously for treating keratin materials, notably α) keratin fibres, notably human keratin fibres such as the eyelashes or the eyebrows, or β) human skin, in particular of the lips, comprising the application to said materials of at least one dispersion (A), for filling skin imperfections such as wrinkles and fine lines or scars, for caring for the skin and/or for making up the skin.

The dispersions (A) of the instant disclosure, notably the hydrogels, are stable. Furthermore, after application of the dispersions (A) to the keratin materials, the material obtained (C) is film-forming, adheres to said materials and has a very significant volumizing effect on the keratin materials once water is added. The water may originate from the addition or else from the support, keratin materials, which is wet. Furthermore, the active agents such as pigments are not impaired in terms of colours during the filling of imperfections, wrinkles, fine lines or scars.

Thus, the dispersion (A) according to the instant disclosure is applied to the skin for the purpose of obtaining a tightening effect on the imperfections of the keratin materials, notably facial skin, the residual polymer material (C) of which after application to said materials and after evaporation of the solvents of the fatty substance (s) iii) and of the water v) if present, is film-forming and adheres to said keratin materials. Mention may notably be made of applications in the field of makeup (lipsticks, mascaras, filling of wrinkles and fine lines, and hiding of imperfections).

The dispersion A, and the process for the treatment of keratin materials as defined above make it possible to obtain a treatment for said materials which is notably resistant to shampoo washing, to sebum, to sweat and/or to water, but also to fatty substances, notably dietary fatty substances such as oils. Furthermore, the dispersion is easy to use in compositions, notably cosmetic compositions, is easy to manufacture and remains stable over time. Specifically, the dispersion (A) in accordance with the instant disclosure makes it possible to obtain deposits of polymer materials (C) on said keratin materials (substrate) that are very resistant to external attacking factors, notably to sebum and to the fatty substances found in food, in particular liquid fatty substances such as plant oils and in particular olive oil. It appears that the skin tightening composition produced with at leat one dispersion (A) is particularly resistant to external attacking factors such as liquid fatty substances, in particular with respect to plant oils such as olive oil. It appears that the makeup produced with at least one dispersion (A), notably lip makeup, is particularly resistant to external attacking factors such as liquid fatty substances, in particular with respect to plant oils such as olive oil. Furthermore, the makeup results obtained with the dispersions (A) are very aesthetic. These dispersions of polymer particles are found at a high solids content in the hydrocarbon-based liquid fatty substance(s) iii). It appears that the application of the dispersions (A) of the instant disclosure to keratin fibres makes it possible to obtain coatings that are persistent with respect to external attacking factors (sunlight, water, shampoo washing, perspiration, sebum, etc.).

The dispersion (A) according to the instant disclosure is emulsifying. It may advantageously comprise organic or inorganic particles, which are compatible with keratin materials, notably the skin, such as silicas and polysaccharides (such as starches) optionally substituted with at least one group chosen from (poly)carboxy(C1-C20)alkyl and (poly)carboxy(C1-C20)alkenyl which may be in granular form, and/or in the form of salts such as octenyl succinates, in the form of salts notably of an alkaline metal such as calcium or aluminium.

Moreover, the polymer materials (C) of the instant disclosure obtained after application to the substrate and evaporation of the solvents [the water v) and/or the fatty substances iii)] from the dispersion (A) have elastic mechanical strength properties, are flexible and espouse the surface, significantly reduce or even fill the surface irregularities and imperfections of said substrate which is in particular flexible, notably the skin, such as the filling of fine lines, wrinkles, comedones, blackheads, scars and other skin imperfections. The polymer materials of the instant disclosure have good affinity for the substrate, do not readily detach from the substrate, but do so sufficiently so that they can be removed, if necessary, by hand, for example with a nail, without this being painful.

For the purposes of the present invention and unless otherwise indicated:
an “alkyl radical” is a linear or branched saturated C1-C8, in particular C1-C6, preferably C1 C4 hydrocarbon-based group such as methyl, ethyl, n-propyl, isopropyl, n-butyl, isobutyl and tert-butyl;
a “(C9-C22)alkyl” radical is a linear or branched, preferably linear, saturated C9-22, in particular C10-C20, preferentially C12-C18, more preferentially C12-C16 hydrocarbon-based group, such as lauryl (C12), myristyl (C14), hexadecyl (C16), stearyl (C18), arachidyl (C20) or behenyl (C22); more particularly, (C9-C18)alkyl is a linear or branched, preferably linear, saturated C9-C18 hydrocarbon-based group;
an “alkylene” radical is a linear or branched divalent saturated C1-C8, in particular C1-C6, preferably C1-C4 hydrocarbon-based group such as methylene, ethylene or propylene;
By “(di) (C1-C4) (alkyl) amino” is an amino radical —NH2; a (C1-C4) alkylamino radical such as methylamino ethylamino; a di (C1-C4) alkylamino radical such as dimethylamino, diethylamino, preferably dimethylamino;
a “cycloalkyl” radical is a cyclic saturated hydrocarbon-based group comprising from 1 to 3 rings, preferably 2 rings, and comprising from 3 to 12 carbon atoms, preferably between 5 and 10 carbon atoms, such as cyclopentyl, cyclohexyl, cycloheptyl, norbornyl, or isobornyl, the cycloalkyl radical possibly being substituted with one or more (C1-C4)alkyl groups such as methyl; preferably, the cycloalkyl radical is an isobornyl group;
a “cyclic” radical is a cyclic saturated or unsaturated, aromatic or non-aromatic hydrocarbon-based group comprising from 1 to 3 rings, preferably 1 ring, and comprising from 3 to 10 carbon atoms, such as cyclohexyl or phenyl;
an “aryl” radical is a monocyclic or fused or non-fused bicyclic, unsaturated cyclic aromatic radical comprising from 6 to 12 carbon atoms; preferably, the aryl group comprises 1 ring and contains 6 carbon atoms, such as phenyl;
an “aryloxy” radical is an aryl-oxy, i.e. aryl-O-, radical, with aryl as defined previously, preferably phenoxy;
an “aryl(C1-C4)alkoxy” radical is an aryl-(C1-C4)alkyl-O- radical, preferably benzoxy;
the term “keratin materials” particularly means the skin (keratinized epithelium) and human keratin fibres such as head hair, the eyelashes, the eyebrows and bodily hair, preferentially head hair, the eyebrows and the eyelashes, even more preferentially head hair;
in the instant disclosure, the term “soluble” monomer refers to any monomer of which the polymer, notably the homopolymer, is soluble, to 5% by weight, at 20° C. and at atmospheric pressure in the liquid hydrocarbon-based medium consisting of liquid hydrocarbon-based fatty substances iii) of the dispersion. The polymer, notably the homopolymer, is completely dissolved in the liquid carbon-based medium, visually at 20° C., i.e. no insoluble deposit or precipitate or agglomerate or sediment is visually noted;
the term “insoluble” monomer refers to any monomer of which the polymer, notably the homopolymer, is not in soluble form, i.e. not fully dissolved at a concentration of greater than 5% by weight at room temperature (20° C.) in said liquid hydrocarbon-based medium consisting of iii) liquid hydrocarbon-based fatty substances. However, the “insoluble” monomers may, as monomers, be soluble in the liquid hydrocarbon-based medium consisting of liquid hydrocarbon-based fatty substances iii) of the dispersion, it being understood that they become insoluble after polymerization;
the term “ethylenic homopolymer” means a polymer derived from the polymerization of identical monomers;
the term “ethylenic copolymer” means a polymer derived from the polymerization of different monomers, in particular at least two different monomers. Preferably, the ethylenic copolymer of the invention is derived from two or three different monomers, more preferentially derived from two different monomers;
the term “ethylenic monomer” means an organic compound including one or more conjugated or non-conjugated unsaturations of >C═C< type, which is capable of polymerizing;
the term “fatty substance” means an organic compound that is immiscible in water at ordinary room temperature (25° C.) and at atmospheric pressure (760 mmHg) (solubility of less than 5%, preferably 1% and even more preferentially 0.1 %). They bear in their structure at least one hydrocarbon-based chain including at least 6 carbon atoms or a sequence of at least two siloxane groups. In addition, the fatty substances are generally soluble in organic solvents under the same temperature and pressure conditions, for instance ethanol, ether, liquid petroleum jelly or decamethylcyclopentasiloxane. These fatty substances are neither polyoxyethylenated nor polyglycerolated. They are different from fatty acids, since salified fatty acids constitute soaps that are generally soluble in aqueous media;
the term “liquid” fatty substance notably refers to a fatty substance that is liquid at 25° C. and 1 atmosphere; preferably, said fatty substance has a viscosity of less than or equal to 7000 centipoises at 20° C.;
the term “hydrocarbon-based” fatty substance means a fatty substance which comprises at least 50% by weight, notably from 50% to 100% by weight, for example from 60% to 99% by weight, or even from 65% to 95% by weight, or even from 70% to 90% by weight, relative to the total weight of said fatty substance, of carbon-based compound, having a global solubility parameter in the Hansen solubility space of less than or equal to 20 (MPa)½, or a mixture of such compounds;
the global solubility parameter δ according to the Hansen solubility space is defined in the article “Solubility parameter values” by Grulke in the book “Polymer Handbook”, 3rd Edition, Chapter VII, pages 519-559, by the relationship δ = ( dD2 + dP2 + dH2)½ in which: - dD characterizes the London dispersion forces arising from the formation of dipoles induced during molecular impacts, - dP characterizes the Debye interaction forces between permanent dipoles, - dH H characterizes the forces of specific interactions (such as hydrogen bonding, acid/base, donor/acceptor, etc.); The definition of solvents in the Hansen three-dimensional solubility space is described in the article by Hansen: The three-dimensional solubility parameters, J. Paint Technol. 39, 105 (1967);
the term “oil” means a fatty substance that is liquid at room temperature (25° C.) and at atmospheric pressure;
the term “hydrocarbon-based oil” means an oil formed essentially from, or even constituted of, carbon and hydrogen atoms, and optionally of oxygen and nitrogen atoms, and not containing any silicon or fluorine atoms. It may contain hydroxy, ester, ether, carboxylic acid, amine and/or amide groups;
the term “volatile oil” means an oil (or non-aqueous medium) that can evaporate on contact with keratin materials, in particular the skin, in less than one hour, at room temperature and at atmospheric pressure. The volatile oil is a volatile cosmetic oil, which is liquid at room temperature, notably having a non-zero vapour pressure, at room temperature and at atmospheric pressure, in particular having a vapour pressure ranging from 0.13 Pa to 40 000 Pa (10-3 to 300 mmHg), preferably ranging from 1.3 Pa to 13 000 Pa (0.01 to 100 mmHg) and preferentially ranging from 1.3 Pa to 1300 Pa (0.01 to 10 mmHg);
the term “non-volatile oil” means an oil with a vapour pressure of less than 0.13 Pa at room temperature and at atmospheric pressure;
the term “silicone oil” means an oil comprising at least one silicon atom and notably at least one Si—O group. The silicone oil may be volatile or non-volatile;
the term “dispersant” refers to a compound which can protect the dispersed particles from agglomerating or flocculating. This dispersant may be a surfactant, an oligomer, a polymer or a mixture of several thereof, bearing one or more functionalities with strong affinity for the surface of the particles to be dispersed; in particular, they can attach physically or chemically to the surface of the pigments. These dispersants also contain at least one functional group that is compatible with or soluble in the continuous medium. Said agent may be charged: it may be anionic, cationic, zwitterionic or neutral;
the term “pigment” refers to any pigment that gives colour to keratin materials, of synthetic or natural origin, the solubility of the pigments in water at 25° C. and at atmospheric pressure (760 mmHg) being less than 0.05% by weight and preferably less than 0.01%;
the term “lake” refers to dyes adsorbed onto insoluble particles, the assembly thus obtained remaining insoluble during use. The inorganic substrates onto which the dyes are adsorbed are, for example, alumina, silica, calcium sodium borosilicate or calcium aluminium borosilicate and aluminium. Among the organic dyes, mention may be made of cochineal carmine;
the term “hair dyes” refers to the oxidation dyes and direct dyes used for dyeing keratin fibres, notably human keratin fibres such as the skin or the hair;
the term “anhydrous” dispersion or composition means a dispersion or composition containing less than 2% by weight of water, or even less than 0.5% of water, and notably free of water; where appropriate, such small amounts of water may notably be provided by ingredients of the composition which may contain residual amounts thereof;
the term “pigments with special effects” refers to pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semitransparent or transparent shade; and
the term “submicron” or “submicronic” refers to pigments having a particle size that has been micronized by a micronization method and having a mean particle size of less than a micrometer (µm), in particular between 0.1 and 0.9 µm, and preferably between 0.2 and 0.6 µm;
the “aryl” or “heteroaryl” radical or the aryl or heteroaryl part of a radical may be substituted with at least one substituent borne by a carbon atom, chosen from:
   a C1-C6 and preferably C1-C4 alkyl radical optionally substituted with one or more radicals chosen from hydroxyl, C1-C2 alkoxy, C2-C4 (poly)hydroxyalkoxy, acylamino, amino substituted with two C1-C4 alkyl radicals, which may be identical or different, optionally bearing at least one hydroxyl group, or the two radicals possibly forming, with the nitrogen atom to which they are attached, a saturated or unsaturated, optionally substituted 5- to 7 membered and preferably 5- or 6-membered heterocycle optionally comprising another nitrogen or non-nitrogen heteroatom;
   halogen;
   hydroxyl;
   C1-C2 alkoxy;
   C2-C4 (poly)hydroxyalkoxy;
   amino;
   an amino radical substituted with one or two identical or different C1-C6 alkyl radicals, optionally bearing at least one hydroxyl group;
   acylamino (—NR—C(O)—R′) in which the radical R is a hydrogen atom or a C1-C4 alkyl radical optionally bearing at least one hydroxyl group and the radical R′ is a C1-C2 alkyl radical;
   carbamoyl ((R)2N—C(O)—) in which the radicals R, which may be identical or different, represent a hydrogen atom or a C1-C4 alkyl radical optionally bearing at least one hydroxyl group;
   alkylsulfonylamino (R′—S(O)2—N(R)—) in which the radical R represents a hydrogen atom or a C1-C4 alkyl radical optionally bearing at least one hydroxyl group and the radical R′ represents a C1-C4 alkyl radical, or a phenyl radical;
   an aminosulfonyl radical ((R)2N—S(O)2—) in which the radicals R, which may be identical or different, represent a hydrogen atom or a C1-C4 alkyl radical optionally bearing at least one hydroxyl group;
   carboxylic in acid or salified (preferably with an alkali metal or a substituted or unsubstituted ammonium) form;
   cyano;
   nitro or nitroso;
   polyhaloalkyl, preferentially trifluoromethyl;
   the cyclic or heterocyclic part of a non-aromatic radical may be substituted with at least one substituent chosen from the following groups: a) hydroxyl; b) C1-C4 alkoxy, C2-C4 (poly)hydroxyalkoxy; c) C1-C4 alkyl;
   alkylcarbonylamino (R—C(O)—N(R′)—) in which the radical R′ is a hydrogen atom or a C1-C4 alkyl radical optionally bearing at least one hydroxyl group, and the radical R is a C1-C2 alkyl radical or an amino radical optionally substituted with one or two C1-C4 alkyl groups, which may be identical or different, themselves optionally bearing at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom;
   alkylcarbonyloxy (R—C(O)—O—) in which the radical R is a C1-C4 alkyl radical or an amino group optionally substituted with one or two identical or different C1-C4 alkyl groups themselves optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
   alkoxycarbonyl (R—G—C(O)—) in which the radical R is a C1-C4 alkoxy radical, G is an oxygen atom or an amino group optionally substituted with a C1-C4 alkyl group itself optionally bearing at least one hydroxyl group, said alkyl radical possibly forming with the nitrogen atom to which they are attached a saturated or unsaturated, optionally substituted 5- to 7-membered heterocycle, optionally comprising at least one other nitrogen or non-nitrogen heteroatom;
a cyclic or heterocyclic radical, or a non-aromatic part of an aryl or heteroaryl radical, may also be substituted with one or more oxo groups;
a hydrocarbon-based chain is unsaturated when it includes one or more double bonds and/or one or more triple bonds;
the term “aryl” means a monocyclic or fused or non-fused polycyclic carbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl;
the term “heteroaryl” means an optionally cationic, 5- to 22-membered, monocyclic or fused or non-fused polycyclic group, comprising from 1 to 6 heteroatoms chosen from nitrogen, oxygen, sulfur and selenium, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof;
the term “heterocyclic” means a 5- to 22-membered, monocyclic or fused or non-fused polycyclic aromatic or non-aromatic radical that may contain one or more unsaturations, including from 1 to 6 heteroatoms chosen from nitrogen, oxygen and sulfur;
the term “heterocycloalkyl” means a saturated heterocyclic radical such as morpholinyl, piperazinyl or piperidyl;
the term “organic or mineral acid salt” more particularly means salts chosen from a salt derived from a halogenated acid such as i) hydrochloric acid HCl, ii) hydrobromic acid HBr, iii) sulfuric acid H2SO4, iv) alkylsulfonic acids: Alk-S(O)2OH such as methylsulfonic acid and ethylsulfonic acid; v) arylsulfonic acids: Ar-S(O)2OH such as benzenesulfonic acid and toluenesulfonic acid; optionally hydroxylated carboxylic acids such as vi) citric acid; vii) succinic acid; viii) tartaric acid; ix) lactic acid; x) acetic acid CH3C(O)OH; xi) alkoxysulfinic acids: Alk-O-S(O)OH such as methoxysulfinic acid and ethoxysulfinic acid; xii) aryloxysulfinic acids such as tolueneoxysulfinic acid and phenoxysulfinic acid; xiii) phosphoric acid H3PO4; xiv) triflic acid CF3SO3H and xv) tetrafluoroboric acid HBF4; more preferentially, the organic or mineral acid salts are chosen from the salts of halogenated acids such as HCl and HBr, and of optionally hydroxylated carboxylic acids such as vi) citric acid; vii) succinic acid; viii) tartaric acid; ix) lactic acid; x) acetic acid CH3C(O)OH;
the term “anionic counterion” means an anion or an anionic group derived from an organic or mineral acid salt which counterbalances the charge of the molecule in question; more particularly, the anionic counterion is chosen from: i) halides such as chloride or bromide; ii) nitrates; iii) sulfonates, including C1-C6 alkylsulfonates: Alk-S(O)2O- such as methylsulfonate or mesylate and ethylsulfonate; iv) arylsulfonates: Ar-S(O)2O- such as benzenesulfonate and toluenesulfonate or tosylate; v) citrate; vi) succinate; vii) tartrate; viii) lactate; ix) alkyl sulfates: Alk-O-S(O)O- such as methyl sulfate and ethyl sulfate; x) aryl sulfates: Alk-O-S(O)O- such as benzene sulfate and toluene sulfate; xi) alkoxy sulfates: Alk-O-S(O)2O- such as methoxy sulfate and ethoxy sulfate; xii) aryloxy sulfates: Ar—O—S(O)2O—, xiii) phosphates O═P(OH)2—O—, O═P(O—)2—OH, O═P(O—)3, HO—[P(O)(O—)]w—P(O)(O—)2 with w being an integer; xiv) acetate; xv) triflate; and xvi) borates such as tetrafluoroborate, xvii) disulfate (O═)2S(O—)2 or SO42-and monosulfate HSO4—; the anionic counterion, derived from an organic or mineral acid salt, ensures the electrical neutrality of the molecule; thus, it is understood that when the anion comprises several anionic charges, then the same anion can serve for the electrical neutrality of several cationic groups in the same molecule or else may serve for the electrical neutrality of several molecules; for example, a molecule which contains two cationic groups may contain either two “singly charged” anionic counterions or contain a “doubly charged” anionic counterion such as (O═)2S(O—)2 or O═P(O—)2—OH;
moreover, the addition salts that may be used in the context of the invention are notably chosen from addition salts with a cosmetically acceptable base such as the basifying agents as defined below, for instance alkali metal hydroxides such as sodium hydroxide, potassium hydroxide, aqueous ammonia, amines or alkanolamines.
the expression “at least one” is equivalent to “one or more”; and
the limits of a range of values are included in that range, notably in the expressions “between... and ...” and “ranging from ... to ...”; and
the expression “inclusive” for a range of concentrations means that the limits of the range are included in the defined interval;
for the purposes of the present invention, the term “skin” means the skin of the body and of the scalp, in particular the skin of the face, the body and the hands;
according to the invention, the term “mature skin” notably means the skin of people at least 40 years old;
according to the invention, the term “very mature skin” notably means the skin of people at least 50, in particular at least 60 or even 65 years old;
the term “wrinkle” means a static skin fold or groove that is visible to the eye, which may be the result of ageing of the keratin materials, notably of the skin, which may be caused by exposure or overexposure to UVA and UVB, smoking, notably of cigarettes, air pollution, a decrease in the oestrogen hormone, oxidative or non-oxidative stress, or a decrease in fatty mass. The wrinkles may be on the face or on the body, preferably on the face. Mention may be made of a) forehead wrinkles (across the forehead), b) frown wrinkles (between the eyebrows or the glabella, running vertically between the two eyebrows), c) crow’s feet wrinkles (periorbital wrinkles, in the outer corner of the eyes in the direction of the ears), d) bitterness wrinkles (cheek-chin or lip-chin groove, wrinkles located between the corners of the mouth down to the chin), e) nasal grooves (vertical wrinkles running from the edges of the nostrils to the corners of the mouth), f) perioral wrinkles, or e) the other wrinkles of the face other than a) to f). In particular, the size of the groove is between 2.5 and 3 mm, the height of the groove is preferably between 0.6 mm and 1 mm, and preferably the angle of the groove is between 20° and 40°, particularly between 30° and 40°;
the term “fine line” means small wrinkles, i.e. the groove size of which is particularly between 0.5 and 1, preferably the height of the groove is between 0.4 and 1 mm, more particularly between 0.5 and 0.8 mm, even more particularly between 0.6 and 0.8 mm and preferably the angle of the groove is between 10° and 40°, particularly between 20° and 30°;
the term “scar” means the part that is visible to the eye of dermal damage following repair of the dermal tissue as a result of an injury, a cut, a burn or an incision, for example;
the term “filling” means the visual reduction of the size of the groove or scar of between % and %;
the term “oxidative stress” refers to all of the damage caused by an increase in oxygen-based free radicals in a subject.

### The Dispersion (A)

The dispersion (A) of the instant disclosure comprises i) at least two polymer particles of different chemical composition preferably stabilized at the surface by ii) at least one stabilizing agent in a preferably anhydrous medium, further containing iii) at at least one fatty substance which is liquid at 20° C. and 1 atmosphere, preferably volatile, hydrocarbon, iv) at least one polyol which is liquid at 20° C. and 1 atmosphere, preferably chosen from (C2-C6) alkanediol and (C2-C6) alkane-triol such as glycerol, and it may also contain v) water and optionally one or more cosmetic active agents (vi) different from iii) and from iv) chosen from ingredients f) to j) as defined previously.

The dispersion (A) thus comprises i) at least two particle (s) of different chemical structure consisting of polymer (s) obtained by polymerization of monomers chosen from ethylenic monomers of:
a₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino,
a₂) poly[oxy(C₁-C₄)alkylene] (C₁-C₄)(alkyl)acrylate,
a₃) ethylenic monomers comprising one or more carboxy, anhydride, phosphoric acid, sulfonic acid groups;
it being understood that each particle results from the polymerization of one or more ethylenic monomer (s) a3).

According to a particular embodiment of the instant disclosure, the dispersion (A) comprises at least one particle i1) consisting of copolymer derived from monomers a1) and / or a2) and a3) and at least one particle i2) of chemical composition different from i1) chosen from a copolymer derived from monomers a1) and / or a2) and a3), a copolymer derived from monomers a3), a homopolymer derived from monomers a3).

According to a particular form of the invention, the dispersion (A) according to the instant disclosure comprises i) at least two particle (s) of different chemical composition comprising one or more polymer (s) chosen from:
a) copolymers of ethylenic monomers of:
   a1) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino; and or
   a2) poly[oxy(C₁-C₄)alkylene] (C₁-C₄)(alkyl)acrylate, and
   a3) ethylenic monomers comprising one or more carboxy, anhydride, phosphoric acid, sulfonic acid groups; or
b) mixtures of:
   b1) at least one copolymer of ethylenic monomers of:
      a1) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino; and or
      a 2) poly[oxy(C₁-C₄)alkylene] (C₁-C₄)(alkyl)acrylate, and
      a3) ethylenic monomers comprising one or more carboxy, anhydride, phosphoric acid, sulfonic acid groups; and
   b2) at least one polymer of ethylenic monomers comprising one or more carboxy, anhydride, phosphoric acid, sulfonic acid groups

In order to obtain the dispersion (A), it is proposed to polymerize particular monomers that are capable of forming the polymer “core” i) in the presence of a polymeric statistical stabilizer ii) comprising in minor amount a part that is soluble ii) and in major amount a part that is insoluble i) in the dispersion medium, i.e. in the mixture iii) of liquid hydrocarbon-based fatty substance(s) and iv) at least one polyol that is liquid at 20° C. and 1 atm, preferably chosen from (C2-C6)alkanediol and (C2-C6)alkanetriol such as glycerol and v) optionally water.

According to a particular embodiment of the instant disclosure the dispersion (A) is such that the particles i) consist of i several copolymers derived from monomers a1), and / or a2) and a3) a) of chemical composition different or mixtures of copolymers b1) with at least one polymer b2), as defined above, and ii) of one or more polymeric stabilizing agent (s) derived from polymer d) and / or copolymers e) as defined above, and iv) one or more polyol (s) liquid at 20° C. and 1 atmosphere, preferably chosen from (C2-C6) alkane-diol and (C2-C6) alkane-triol such as glycerol, said dispersion possibly further comprising one or more cosmetic active agent (s) vi) as defined above.

According to a particular embodiment of the invention, the dispersion (A) is such that the particle(s) i) consist of an ethylenic polymeric core obtained from homopolymers a) or from copolymers b) or c), as defined previously, and ii) of one or more polymeric surface stabilizers obtained from homopolymer d), and the copolymers e) as defined previously, and iv) one or more polyols that are liquid at 20° C. and 1 atm, preferably chosen from (C2 C6)alkanediol and (C2-C6)alkanetriol such as glycerol.

The dispersions (A) according to the invention consist of particles, which are generally spherical, and ii) of at least one surface-stabilized polymer, in a mixture iii) of liquid hydrocarbon-based fatty substances, v) optionally of water, in the presence iv) of at least one polyol that is liquid at 20° C. and 1 atm, preferably chosen from (C2-C6)alkanediol and (C2-C6)alkanetriol such as glycerol. said dispersion possibly further comprising one or more cosmetic active (s) (vi) as defined above.

According to another particular embodiment of the instant disclosure the dispersion (A) is such that the particles i) consist of several copolymers derived from monomers a1), and / or a2) and a3) a) of chemical composition different or mixtures of copolymers b1) with at least one polymer b2), as defined above, and ii) of one or more polymeric stabilizing agent (s) derived from polymer d) and / or copolymers e) as defined above, and iv) one or more polyol (s) liquid at 20° C. and 1 atmosphere, preferably chosen from (C2-C6) alkane-diol and (C2-C6) alkane-triol such as glycerol, as well as v) water, said dispersion possibly further comprising one or more cosmetic active (s) (s) vi) as defined above.

According to another particular embodiment of the instant disclosure the dispersion (A) is present from at least about 5 wt.% based on the total weight of the skin tightening composition.

The stabilizer(s) are particularly present in the dispersion (A) in an amount of between 0.01 % and 30% by weight relative to the total weight of the dispersion (A), more particularly between 0.1% and 20% by weight, preferably between 0.5% and 10% by weight, more preferentially between 0.7% and 4.5% by weight relative to the total weight of said dispersion (A).

According to a particular embodiment of the instant disclosure, the amount of stabilizer(s) ii) is between 0.05% and 30% by weight relative to the total weight of said composition (A) without v) water, preferably between 0.2% and 20% by weight, notably between 2% and 15% by weight, more particularly between 2% and10% by weight, more preferentially between 2.5% and 5% by weightrelative to the total weight of said dispersion (A) without v) water.

According to a particular embodiment of the instant disclosure, the amount of stabilizer(s) ii) is between0.01% and 20% by weight relative to the total weight of said composition (A) comprising water, preferably between0.1% and 15% by weight, notably between 0.9% and 10% by weight, more particularly between 1% and 5% by weight, more preferentially between 2 and 3.5% by weight relative to the total weight of said dispersion (A) comprising v) water.

According to a particular embodiment of the instant disclosure, ii) the stabilizer(s) are present in an amount of between 5% and 40% by weight relative to the total weight of the ingredients i) + ii), more particularly between 8% and 30% by weight, preferably between 9.8% and 12.5% by weight relative to the total weight of the ingredients i) + ii).

Preferably, the monomers i) that are capable of forming the polymeric core of the particle are chosen from monomers that are insoluble in the liquid hydrocarbon-based medium consisting of iii) liquid hydrocarbon-based fatty substances. The insoluble monomers notably represent 100% by weight relative to the total weight of the monomers forming the polymeric core of the particle.

Preferably, the constituent monomers of the particles i) are chosen from monomers insoluble in the liquid hydrocarbon-based medium consisting of iii) liquid fatty substance (s) hydrocarbon (s). The insoluble monomers represent in particular 80% to 100% by weight, preferably 90% to 100% by weight, more preferably 95% to 100% by weight, even more preferably 100% by weight, of the total weight of the monomers forming the particles(s).

### Polymer Particles I

According to a particular embodiment of the instant disclosure the particles i) of the dispersion (A) is (are) made of at least two, preferably two polymer (s) chosen from
a) the copolymers ethylenic monomers of:
   a₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino; and / or
   a₂) poly[oxy(C₁-C₄)alkylene] (C₁-C₄)(alkyl)acrylate, and
   a₃) ethylenic monomers comprising one or more carboxy, anhydride, phosphoric acid, sulfonic acid groups
   According to another particular embodiment of the instant disclosure, the particles i) of the dispersion (A) is (are) made up of several polymer (s) chosen from among
b₁) at least one copolymer of ethylenic monomers of:
   a₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino; and / or
   a₂) poly[oxy(C₁-C₄)alkylene] (C₁-C₄)(alkyl)acrylate, and
   a₃) ethylenic monomers comprising one or more carboxy, anhydride, phosphoric acid, sulfonic acid groups; and
b₂) at least one polymer of ethylenic monomers comprising one or more carboxy, anhydride, phosphoric acid or sulfonic acid groups.

Preferably, the ethylenic monomers comprising one or more carboxy, anhydride, phosphoric acid, sulfonic acid a3) groups are chosen from ethylenic monomers comprising one or more carboxy groups, more preferably (C1-C4) (alkyl) acrylic acids such as (meth) acrylic acid, in particular acrylic acid. Preferably, the ethylenic monomers of (C1-C4)alkyl (C1-C4)(alkyl)acrylate in which (C1-C4) alkyl denote (meth) acrylate of (C1-C4) alkyl such as ethyl (meth) acrylate or ( methyl meth) acrylate, in particular methyl acrylate and ethyl acrylate.

Preferably, the ethylenic monomers of (C₁-C₄)alkyl (C₁-C₄)(alkyl)acrylate in which the (C1-C4) alkyl group or groups are substituted by one or more groups chosen from hydroxy, (di) ((C1-C4) alkyl) amino are chosen from ethylenic monomers of (C1-C4) (C₁-C₄) alkyl (alkyl) acrylate substituted with a hydroxy group or with a (di) ((C1-C4) group ) alkyl) amino.According to one embodiment, the ethylenic monomers of (C1-C4) (C₁-C₄) alkyl (alkyl) acrylate in which the (C1-C4) alkyl group (s) are substituted by one or more hydroxy groups , are substituted by a hydroxy group such as 2-hydroxyethyl (meth) acrylate, in particular 2-hydroxyethyl acrylate (HEA).

According to another embodiment, the ethylenic monomers of (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino, are substituted by a di (C1-C4) alkyl) amino group such as a dimethylamino group such as 3-(dimethylamino) propyl (meth) acrylate, 2- (dimethylamino) ethyl (meth ) acrylate.

According to a particular embodiment of the instant disclosure, the particles i) of the dispersion (A) contain particles A1 consisting of copolymers of ethylenic monomers:
a₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino; and or
a₂) poly[oxy(C₁-C₄)alkylene] (C₁-C₄)(alkyl)acrylat, and
a₃) ethylenic monomers comprising one or more carboxy, anhydride, phosphoric acid, sulfonic acid,
preferably consisting of copolymers of ethylenic monomers a₁) and a₃), or a₂) and a₃), more preferably a₁) and a₃).

According to a particular embodiment of the instant disclosure, the particles i) of the dispersion (A) contain particles A′1 consisting of copolymers of ethylenic monomers a′₁) (C₁-C₄)alkyl (C₁-C₄)(alkyl)acrylate and a₃) ethylenic monomers comprising one or more carboxy, anhydride, phosphoric acid, sulfonic acid,

According to another particular embodiment of the instant disclosure, the particles i) of the dispersion (A) contain particles A′2 consisting of copolymers of ethylenic monomers a″₁) (C1-C4) (alkyl) (C1-C4) alkyl acrylate substituted with one or more groups chosen from hydroxy, (di) ((C1-C4) alkyl) amino, preferably substituted with a hydroxy group such as 2-hydroxyethylacrylate (HEA) and a₃) monomers ethylenic comprising one or more carboxy, anhydride, phosphoric acid, sulfonic acid.

According to a preferred embodiment, the particles i) of the dispersion (A) contain particles A′1 and particles A′2 as described above and more preferably the particles i) of the dispersion (A) are a mixture of particles A′1 and A′2, in particular in a mass ratio (mass of particles A′1 / mass of particles A′2) of between 0.3 and 3, more particularly between 0.5 and 2, 8, even more preferably between 0.6 and 2.

According to another particular embodiment of the instant disclosure the particles i) of the dispersion (A) is (are) made up of a mixture of b₁) at least one copolymer of ethylenic monomers of a₁) and / or a₂) and a₃) as described above and b₂) at least one polymer of ethylenic monomers comprising one or more carboxy, anhydride, phosphoric acid or sulfonic acid groups.

According to another particular embodiment of the instant disclosure the particles i) of the dispersion (A) is (are) made up of a mixture of b₁) at least one copolymer of ethylenic monomers of a₁) and a₃) as described above and b₂) at least one polymer of ethylenic monomers comprising one or more carboxy, anhydride, phosphoric acid or sulfonic acid groups.

According to a particular form of the instant disclosure, b₂) denotes a homopolymer of ethylenic monomers comprising one or more carboxy, or anhydride, or phosphoric acid or sulfonic acid groups, preferably comprising a carboxy group, more preferably acid ( meth) acrylic and even more preferably acrylic acid.

The particles i) can be crosslinked or uncrosslinked.

According to one embodiment of the instant disclosure, the particles i) contain ethylenic copolymers A′1 resulting from the polymerization of monomer of formula (I) with ethylenic monomers comprising one or more carboxy, anhydride, phosphoric acid, sulfonic acid a3 groups. ).

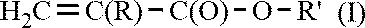

in which formula (I):
R represents a hydrogen atom or a (C1-C4)alkyl group such as methyl, and
R′ represents a (C1-C4)alkyl group such as methyl or ethyl, preferably a C1-C4 alkyl acrylate such as methyl acrylate.

According to a particular embodiment of the instant disclosure the (C1-C4) (C1-C4) (alkyl) acrylate monomers denote the C1-C4 alkyl (meth) acrylate monomers preferably chosen from methyl (meth) acrylate, ethyl (meth) acrylate, n-propyl (meth) acrylate, isopropyl (meth) acrylate, n-butyl (meth) acrylate, (meth) acrylate isobutyl, tert-butyl (meth) acrylate and more preferably chosen from methyl (meth) acrylate, ethyl (meth) acrylate and even more preferably chosen from methyl acrylate and ethyl acrylate.

According to an advantageous embodiment of the instant disclosure, the dispersion (A) comprises from 60% to 99% by weight, in particular from 70% to 99% by weight, preferably from 80% to 95% by weight, in particular from 85% to 93% by weight of monomers constituting the particles i) relative to the total weight of polymers contained in said dispersion (A).

Preferably, the monomers capable of forming the particles i) are chosen from monomers which are insoluble in the hydrocarbon-based liquid fatty substance (s) iii) of the dispersion (A).

The insoluble monomers preferably represent from 80 to 100% by weight, more preferably from 90 to 100% by weight, even more preferably from 95% to 100% by weight and even more particularly 100% by weight, of the total weight. monomers forming the particles i).

### Ethylenic Monomers Bearing an Acid, Anhydride or Aryl Function

The particles i) comprise ethylenic polymers comprising or consisting of ethylenic monomers a₃) comprising one or more carboxy, anhydride, phosphoric acid or sulfonic acid groups.

More particularly the ethylenic monomers a₃) comprising one or more carboxy, anhydride, phosphoric acid or sulfonic acid groups are chosen from (1), (2), (3) and (4):
(1) R1(R2)C═C(R3)—Acid with R¹, R² and R³ representing a hydrogen atom or a CO₂H, H₂PO₄ or SO₃H group, and Acid representing a carboxyl, phosphoric acid or sulfonic acid group, preferably carboxyl, and also the organic or mineral base salts thereof such as the alkali metal or alkaline-earth metal salts, such as C(O)ONa or C(O)OK, preferably (1) represents (5) H2C═C(R)—C(O)—O—H with R representing a hydrogen atom or a (C₁-C₄)alkyl group such as methyl, and also the organic or mineral base salts thereof such as the alkali metal or alkaline-earth metal salts, such as Na or K;
(2) H2C═C(R)—C(O)—N(R′)—Alk—Acid with R and R′, which may be identical or different, representing a hydrogen atom or a (C₁-C₄)alkyl group; Alk represents a (C₁-C₆)alkylene group optionally substituted with at least one group chosen from Acid as defined previously and hydroxyl; and Acid is as defined previously, preferably carboxyl or sulfonic acid, and also the organic or mineral base salts thereof such as the alkali metal or alkaline-earth metal salts such as C(O)ONa or C(O)OK;
(3) Ar—(Ra)C═C(R^{b})—R^{c} with R^{a}, R^{b} and R^{c}, which may be identical or different, representing a hydrogen atom or a (C₁-C₄)alkyl group, and Ar representing an aryl group, preferably benzyl, optionally substituted with at least one acid group CO₂H, H₂PO₄ or SO₃H, preferably substituted with a CO₂H or SO₃H group, and also the organic or mineral base salts thereof such as the alkali metal or alkaline-earth metal salts such as C(O)ONa or C(O)OK;
(4) maleic anhydride of formulae (4a) and (4b):

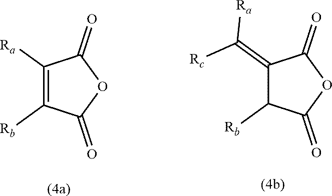

in which formulae (4b) and (4b) Rₐ, R_{b} and R_{c}, which may be identical or different, represent a hydrogen atom or a (C₁-C₄)alkyl group; preferably, Rₐ, R_{b}, and R_{c} represent a hydrogen atom. Preferentially, the ethylenically unsaturated anhydride monomer of the invention is of formula (4b) and more preferentially is maleic anhydride; and
more particularly, the ethylenic monomer a3) comprising one or more carboxy, anhydride, phosphoric acid or sulfonic acid groups is chosen from (1) and (4), in particular (5) and more particularly (5) and even more preferably acrylic acid.

According to another particular embodiment of the instant disclosure, the polymer constituting the particles i) and notably A′1 and/or b₁) is an ethylenic acrylate copolymer derived from the polymerization:
at least one monomer a₁) of formula (I) as defined previously, preferably a C₁-C₄ alkyl acrylate such as methyl acrylate or ethyl acrylate; and
at least one monomer a₃) of formula (5) and also the organic or mineral base salts thereof such as the alkali metal or alkaline-earth metal salts such as Na or K:

According to this embodiment, the amount of acrylic acid ranges from 0.01 % to 30% by weight relative to the total weight of copolymer A′1 or b₁, preferably between 0.1 % and 20% by weight relative to the weight of the polymer(s) of the particles i). More particularly A′1 or b₁), is in particular a copolymer resulting from the copolymerization of acrylic acid with one or more C1-C4 alkyl (meth) acrylate monomers, in particular chosen from methyl (meth) acrylate, and ethyl (meth) acrylate.

According to another preferred embodiment of the invention, the polymer constituting the particles i) and notably A′1) and /or b₁) is an ethylenic acrylate copolymer derived from the polymerization:
of at least two different monomers a1): of formula (I) as defined previously, preferably a C1-C4 alkyl (meth)acrylate such as methyl (meth)acrylate or ethyl (meth)acrylate; and
of a monomer a₃) of formula (5) as defined previously, as well as its salts of organic or inorganic bases such as alkalis or alkaline-earth metals such as Na or K, preferably acrylic acid. Even more preferably A′1 and / or b₁) results from the polymerization of methyl acrylate, ethyl acrylate and acrylic acid. According to one embodiment, the ethylenic monomer a₃) comprising one or more carboxy, anhydride, phosphoric acid, sulfonic acid groups is chosen from crotonic acid, maleic anhydride, itaconic acid, fumaric acid, maleic acid, styrenesulfonic acid, vinylbenzoic acid, vinylphosphoric acid, acrylic acid, methacrylic acid, acrylamidopropanesulfonic acid, acrylamidoglycolic acid, and their salts, even more preferably the monomer ethylenic comprising one or more carboxy, anhydride, phosphoric acid, sulfonic acid represents acrylic acid and its salts.

According to one embodiment of the invention, the dispersion comprises at least 2 types of different particles, preferably 2 to 5 types of particles, more preferably two types of different particles.

According to one embodiment of the invention, the dispersion comprises at least 2 types of particles i) different, preferably 2 types of particles which are copolymers of different chemical structure derived from monomers a₁) and / or a₂) and a₃), in particular which are copolymers of different chemical structure obtained by polymerization of monomers a₁) and a₃).

According to this embodiment, the particles i) different, preferably comprise ethylenic copolymers (IA) of:
a″₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino, preferably hydroxy and / or
a₂) oly[oxy(C₁-C₄)alkylene] (C₁-C₄)(alkyl)acrylate, and
a₃) ethylenic monomers comprising one or more carboxy, anhydride, phosphoric acid, sulfonic acid, preferably carboxy groups; preferably ethylenic copolymers of a′1) and a₃).

More particularly the ethylenic monomers a₃) comprising one or more carboxy, anhydride, phosphoric acid, sulfonic acid groups are chosen from (1), (2), (3), and (4) as defined above and denote in particular (5) as well as its salts of organic or inorganic bases such as alkalis or alkaline earth metals such as Na or K.

According to this particular embodiment of the invention, the particles i) different, preferably comprise i) ethylenic copolymers (I′A) resulting from the polymerization:- a‴₁ of at least one monomer of formula (I′) H₂C═C(R)—C(O)—O—R″, in which formula (I′):
R represents a hydrogen atom or a (C₁-C₄)alkyl group such as methyl, and
R″ represents a (C₁-C₄)alkyl group substituted with one or more hydroxyl groups (preferably, the alkyl group is substituted at the end of the chain with a hydroxyl group), such as hydroxymethyl or 2-hydroxyethyl; preferably, (I′) represents a C₁-C₄ hydroxyalkyl (meth)acrylate such as hydroxyethyl acrylate; and
a₃″) of at least one monomer of formula (5) as defined previously, as well as its salts of organic or inorganic bases such as alkalis or alkaline-earth metals such as Na or K,

According to yet another particular embodiment of the invention, the different particle (s) i) preferably comprise an ethylenic copolymer (IB) resulting from the polymerization:
a₂″) of at least one monomer of formula (I′) H₂C═C(R)—C(O)—O—[ALK—O]ₚ—R′, in which formula (I″):
R represents a hydrogen atom or a (C₁-C₄)alkyl group such as methyl, and
R′ represents a (C₁-C₄)alkyl group such as methyl or ethyl, preferably a C₁-C₄ alkyl acrylate such as methyl acrylate;
ALK represents a (C₁-C₆)alkylene group optionally substituted with at least one group chosen from Acid as defined previously and hydroxyl; preferably, ALK represents a (C₁-C₄)alkylene group such as ethylene, propylene, butylene or isobutylene; more preferentially, ALK represents an ethylene group;
p represents an integer greater than or equal to 1 and less than or equal to 100; and
a₃″) of at least one monomer of formula (5) as defined previously, as well as its salts of organic or inorganic bases such as alkalis or alkaline-earth metals such as Na or K, preferably acrylic acid..

According to this embodiment, the different particle (s) i) preferably comprise ethylenic copolymers (IC) obtained from the polymerization of:
a ″ ₁) (C1-C4) (alkyl) acrylate (C1-C4) alkyl monomers, preferably of formula (I) defined above; and / or
a₂) poly[oxy(C₁-C₄)alkylene] (C₁-C₄)(alkyl)acrylate, and
a₃) ethylenic monomers comprising one or more carboxy, anhydride, phosphoric acid, sulfonic acid groups, preferably chosen from (1), (2), (3), and (4) as defined above, more particularly of formula (5) ) as well as its salts of organic or inorganic bases such as alkalis or alkaline-earth metals such as Na or K and even more preferably acrylic acid, preferably ethylenic copolymers (IC) resulting from the polymerization of monomers a ″ ‘1) and a3), the monomers a’″ 1) preferably denoting C1-C4 alkyl (meth) acrylates, in particular ethyl (meth) acrylate, methyl (meth) acrylate more preferably methyl acrylate, ethyl acrylate.

More preferably, the copolymers (IC) result from the polymerization of several different monomers a″ ‘₁) and of monomers a₃), even more preferably from ethyl (meth) acrylate with methyl (meth) acrylate and monomers a₃), more particularly methyl acrylate with ethyl acrylate and monomers a₃).

According to another embodiment of the invention, the dispersion comprises at least 2 types of different particles, preferably 2 types of different particles which are chosen from copolymers obtained by polymerization of monomers a₁) and a₃) as defined. previously, and b₂) polymers of ethylenic monomers comprising one or more carboxy, anhydride, phosphoric acid, sulfonic acid, preferably carboxy groups.

According to this embodiment, the copolymers are preferably chosen from copolymers (IC) as described above.

The polymers of ethylenic monomers b₂) preferably denote the homopolymer (ID), more preferably a homopolymer of monomers chosen from (1), (2), (3), and (4) as defined above, more particularly of monomers of formula (5) as well as its salts of organic or inorganic bases such as alkalis or alkaline-earth metals such as Na or K and even more preferably of acrylic acid.

The polymer particle (s) i), of the dispersion (A) a (have) preferably a number-average size is greater than 50 nm.

The final size of the particles i) of structure (ID) included in the dispersion (A) is preferably greater than 50 nm. In particular, a number-average size ranging from 50 nm to 600 nm; more particularly ranging from 100 nm to 500 nm, even more particularly ranging from 150 nm to 400 nm.

The final size of the particles i) of structure (IA), (IB), (IC) or (IE) included in dispersion (A) is preferably greater than 100 nm. In particular, a number-average size ranging from 100 nm to 100 µm; more particularly ranging from 500 nm to 10 µm.

The mean size of the particles is determined via conventional methods known to those skilled in the art. A Malvern brand NanoZS model laser particle size analyser (which is particularly suitable for submicron dispersions) makes it possible to measure the size distribution of these samples. The operating principle of this type of machine is based on dynamic light scattering (DLS), also known as quasi-elastic light scattering (QELS) or photon correlation spectroscopy (PCS). It is also possible to determine the particle size by transmission microscopy.

The sample is pipetted into a disposable plastic tank (four transparent faces, side length of 1 cm and volume of 4 mL) placed in the measuring cell. The data are analysed on the basis of a cumulant fit method which leads to a monomodal particle size distribution characterized by an intensity-weighted mean diameter d (nm) and a size polydispersity factor Q. The results may also be expressed in the form of statistical data such as D10; D50 (median), D90 and the mode.

Other particle size techniques make it possible to obtain this type of information, such as analysis of the individual tracking of particles (Nanoparticle Tracking Analysis, NTA), laser scattering (LS), acoustic extinction spectroscopy (AES) spatial-filter Doppler velocimetry or image analysis.

### The Stabilizer(s) II

The dispersion (A) according to the instant disclosure also comprises one or more stabilizers ii). Preferably, only one type of stabilizer ii) is used in the invention.

According to another particular embodiment of the invention, the stabilizer(s) ii) are chosen from d) ethylenic homopolymers of (C3-C12)cycloalkyl (C1-C6)(alkyl)acrylate monomers, preferably (C3-C12)cycloalkyl (meth)acrylate ethylenic homopolymers; more particularly ethylenic homopolymers derived from the polymerization of monomers of formula: H₂C═C(R)—C(O)—O—R″ with R as defined previously, and R″ representing a (C₅-C₁₀)cycloalkyl group such as norbornyl or isobornyl, preferably isobornyl.

More particularly, the stabilizer(s) ii) consist of ethylenic polymers chosen from ethylenic homopolymers d) or d′) derived from the polymerization of monomers having the following formula:

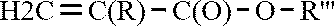

with R representing a hydrogen atom or a (C₁-C₄)alkyl group such as methyl, and R‴ representing a (C₅-C₁₀)cycloalkyl group such as norbornyl or isobornyl, preferably isobornyl

According to a particular embodiment of the invention, the stabilizer(s) ii) are chosen from e) copolymers of ethylenic monomers of e₁) (C₃-C₁₂)cycloalkyl (C₁-C₆)(alkyl)acrylate, and of e₂) (C₁-C₄)alkyl (C₁-C₄)(alkyl)acrylate, preferably copolymers of (C₃-C₁₂)cycloalkyl (meth)acrylate and of (C₁-C₄)alkyl (meth)acrylate.

According to another embodiment, the stabilizer(s) ii) are chosen from e′) ethylenic copolymers of e₁) (C3-C12)cycloalkyl (C1-C6)(alkyl)acrylate, and of e₂) (C1-C4)alkyl (C1-C4)(alkyl)acrylate, preferably copolymers e1) of (C3-C12)cycloalkyl (meth)acrylate and e₂) of (C1-C4)alkyl (meth)acrylate.

More particularly, the stabilizer(s) ii) are chosen from the ethylenic copolymers e) of monomers of formula (IV) and of monomers of formula (III):

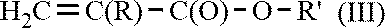

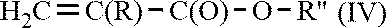

in which formulae (III) and (IV):
R, which may be identical or different, represents a hydrogen atom or a (C₁-C₄)alkyl group such as methyl;
R′, which may be identical or different, represents a (C₁-C₄)alkyl group such as methyl or ethyl; and
R″ represents a (C₅-C₁₀)cycloalkyl group such as norbornyl or isobornyl, preferably isobornyl.

According to another particular embodiment of the instant disclosure, R″ represents a (C5-C10)cycloalkyl group such as norbornyl or isobornyl, preferably isobornyl.

According to a particular embodiment, the stabilizer(s) ii) of the invention consist of ethylenic copolymers e) chosen from e₁) polymers of (C₃-C₁₂)cycloalkyl (C₁-C₆)(alkyl)acrylate monomers notably of formula (IV) and of formula (III) as defined previously.

More particularly, the stabilizer(s) ii) of the invention consist of ethylenic copolymers e) chosen from e′₁) norbornyl (meth)acrylate or isobornyl (meth)acrylate, preferably isobornyl (meth)acrylate, and e′₂) methyl (meth)acrylate or ethyl (meth)acrylate.

According to another embodiment, the stabilizer(s) ii) are chosen from the ethylenic copolymers e) derived from the polymerization e1) of a monomer of formula (IV) as defined previously and e₂) of two different monomers of formula (III) as defined previously.

Preferentially, the stabilizer(s) ii) are chosen from copolymers derived from the polymerization of e₁) one monomer of formula (IV) as defined previously notably chosen from isobornyl (meth)acrylate and e₂) of two different monomers of formula (III) as defined previously, notably different C1-C4 alkyl (meth)acrylates, preferably methyl and ethyl acrylate.

Particularly, the stabilizer ii) is chosen from d) homopolymers of (C3 C12)cycloalkyl (C1-C6)(alkyl)acrylate monomers; and e) statistical copolymers of e1) (C3 C12)cycloalkyl (C1-C6)(alkyl)acrylate and e2) (C1-C4)alkyl (C1-C4)(alkyl)acrylate with a weight ratio e₁/e₂ of greater than 4. Advantageously, said weight ratio ranges from 4.5 to 19. More advantageously, said weight ratio e₁/e₂ ranges from 5 to 15 and more preferentially said weight ratio ranges from 5.5 to 12.

More particularly, the stabilizer ii) is a polymer chosen from d′) isobornyl (meth)acrylate homopolymer and e) statistical copolymers of e1′) isobornyl (meth)acrylate and of e2) C1-C4 alkyl (meth)acrylate preferably present in an isobornyl (meth)acrylate/C1 C4 alkyl (meth)acrylate weight ratio (e1/e2) of greater than 4. Advantageously, said weight ratio e1′/e2′ ranges from 4.5 to 19. Advantageously, said weight ratio e1′/e2′ ranges from 5 to 15 and more preferentially said weight ratio e1′/e2′ ranges from 5.5 to 14.

The stabilizing agent (s) ii) as defined above preferably comprise 80% to 100% by weight of monomer soluble in liquid hydrocarbon fatty substances iii), in particular from 85% to 95% by weight of soluble monomer, alone or as a mixture, relative to the total weight of monomers of the stabilizing agent (s). The stabilizer (co) polymer (s) ii) comprises (s) particularly between 0% and 20% by weight, in particular between 5% and 15% by weight of monomer insoluble in liquid hydrocarbon fatty substances iii) , alone or as a mixture relative to the total weight of monomers of the stabilizing agent (s).

Preferably, the stabilizer(s) ii) and the particle(s) i) have a number-average molecular weight (Mn) of between 1000 and 1 000 000 g/mol, notably between 5000 and 500 000 g/mol and better still between 10 000 and 300 000 g/mol.

The dispersion (A) according to the instant disclosure is finally formed from polymer particles, of relatively large diameter, i.e. preferably greater than 50 nm, and leads to deposits of film-forming compounds (C) which are resistant to fatty substances at room temperature (25° C.), and which are notably advantageous for makeup and/or haircare applications.

According to a particular embodiment of the invention, the dispersion (A) includes from 5% to 40% by weight, in particular from 7% to 20% by weight, notably from 8% to 15% by weight and preferably from 9% to 13% by weight of (C3-C12)cycloalkyl (C1 C6)(alkyl)acrylate monomers d) or e1), relative to the total weight of polymers contained in said dispersion.

### The Hydrocarbon-Based Liquid Fatty Substance(s) III

The dispersion of polymer particles (A) according to the instant disclosure also comprises iii) one or more hydrocarbon-based liquid fatty substances in which said particles are dispersed.

According to a particular embodiment, the hydrocarbon-based liquid fatty substance(s) iii) of the invention are chosen from hydrocarbons, in particular alkanes, oils of animal origin, oils of plant origin, glycerides or fluorinated oils of synthetic origin, fatty alcohols, esters of fatty acids and/or of fatty alcohols, non-silicone waxes, and silicones; in particular, the liquid hydrocarbon-based fatty substance(s) are hydrocarbon-based oils, which are preferably volatile, or are a mixture of different volatile oils, , more particularly isododecane.

The hydrocarbon-based liquid fatty substances iii) are notably chosen from C₆ -C₁₆ hydrocarbons or hydrocarbons comprising more than 16 carbon atoms and up to 50 carbon atoms, preferably between C₆ and C₁₆, and in particular alkanes, oils of animal origin, oils of plant origin, glycerides, fatty alcohols, fatty acid and/or fatty alcohol esters, and silicones.

It is recalled that, for the purposes of the instant disclosure, the fatty alcohols, fatty esters and fatty acids more particularly contain one or more linear or branched, saturated or unsaturated hydrocarbon-based groups comprising 6 to 50 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ alkanes, they are linear or branched, and possibly cyclic. Examples that may be mentioned include hexane, decane, undecane, dodecane, tridecane or isoparaffins, such as isohexadecane, isodecane or isododecane, and their mixtures such as the combination of undecane and tridecane such as for example CETIOL UT®, or mixtures of C₉-C₁₂ alkanes, preferably of natural origin, in particular linear or branched alkanes, C₉-C₁₂. This latter mixture is in particular known under the name INCI C9-C12 ALCANE E511470, CAS 68608-12-8, VEGELIGHT SILK® marketed by BioSynthls. This mixture of volatile, biodegradable, volatile oils obtained from coconut oil (viscosity is 0.9-1.1 cSt (40° C.) and a flash point at 65° C.).

The linear or branched hydrocarbons containing more than 16 carbon atoms may be chosen from liquid paraffins, petroleum jelly, liquid petroleum jelly, polydecenes, and hydrogenated polyisobutene such as Parleam®.

Among the hydrocarbon-based liquid fatty substances iii) having an overall solubility parameter according to the Hansen solubility space of less than or equal to 20 (MPa)½, mention may be made of oils, which may be chosen from natural or synthetic, hydrocarbon-based oils, which are optionally branched, alone or as a mixture.

According to a very advantageous embodiment, the dispersion (A) according to the invention comprises one or more liquid fatty substances which are one or more hydrocarbon-based oils. The hydrocarbon-based oil(s) may be volatile or non-volatile.

According to a preferred embodiment of the instant disclosure, the liquid hydrocarbon-based fatty substance(s) are hydrocarbon-based oils which are volatile or are a mixture of different volatile oils.

According to a preferred embodiment of the instant disclosure, the fatty substance (s) are linear or branched hydrocarbon oils which are volatile in particular chosen from undecane, dodecane, isododecane, tridecane, and their mixture of ‘different, volatile oils preferably comprising isododecane in the mixture, or a mixture of undecane and tridecane

According to another particular embodiment, the liquid fatty substance (s) c) are a mixture of a volatile hydrocarbon oil and a non-volatile hydrocarbon oil, the mixture of which preferably comprises dodecane or isododecane as volatile oil.

According to another advantageous embodiment of the instant disclosure, the fatty substance (s) of the invention liquid (s) hydrocarbon (s) is (are) a mixture of non-volatile oil (s) and the volatile oil (s) preferably the mixture comprises, as volatile oil, undecane, dodecane, isododecane, tridecane, more preferably isododecane. As volatile and non-volatile oil mixture, mention may be made of a mixture of isododecane and isononyl isononanoate.

More preferably when the fatty substance (s) are a mixture of volatile and non-volatile oil, the amount of volatile oil is greater than the amount of non-volatile oil

According to another particular embodiment, the hydrocarbon-based liquid fatty substance(s) iii) are a mixture of a volatile oil and a non-volatile oil such as an isododecane/octyldodecanol mixture or an isododecane / isononyl isononanoate mixture..

The hydrocarbon-based oil may be chosen from:
hydrocarbon-based oils containing from 8 to 14 carbon atoms, and notably:
   branched C8-C14 alkanes, for instance C8-C14 isoalkanes of petroleum origin (also known as isoparaffins), for instance isododecane (also known as 2,2,4,4,6-pentamethylheptane), isodecane and, for example, the oils sold under the trade names Isopar or Permethyl,
   linear alkanes, for instance n-dodecane (C12) and n-tetradecane (C14) sold by Sasol under the references, respectively, Parafol 12-97 and Parafol 14-97, and also mixtures thereof, the undecane-tridecane mixture, mixtures of n-undecane (C11) and of n-tridecane (C13) obtained in examples 1 and 2 of patent application WO 2008/155 059 from the company Cognis, and mixtures thereof,
   hydrocarbon-based oils of plant origin such as triglycerides constituted of fatty acid esters of glycerol, the fatty acids of which may have chain lengths ranging from C4 to C24, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil or musk rose oil; shea butter; or else caprylic/capric acid triglycerides, for instance those sold by the company Stearinerie Dubois or those sold under the names Miglyol 810®, 812® and 818®,
   synthetic ethers containing from 10 to 40 carbon atoms,
   linear or branched hydrocarbons of mineral or synthetic origin, such as petroleum jelly, polydecenes, hydrogenated polyisobutene such as Parleam®, squalane and liquid paraffins, and mixtures thereof,
   esters such as oils of formula R1C(O)—O—R2 in which R1 represents a linear or branched fatty acid residue including from 1 to 40 carbon atoms and R2 represents a, notably branched, hydrocarbon-based chain containing from 1 to 40 carbon atoms, on condition that R1 + R2 ≥ 10, for instance purcellin oil (cetostearyl octanoate), isopropyl myristate, isopropyl palmitate, C12 to C15 alkyl benzoates, hexyl laurate, diisopropyl adipate, isononyl isononanoate, 2-ethylhexyl palmitate, isostearyl isostearate, 2-hexyldecyl laurate, 2 octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
   fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2 undecylpentadecanol.

In addition to the liquid hydrocarbon-based fatty substance, the dispersion (A) may comprise a silicone oil. If the silicone oil is in the dispersion (A), it is preferably in an amount which does not exceed 10% by weight relative to the weight of the dispersion (A), more particularly in an amount of less than 5% and more preferentially 2%.

In particular, the dispersion (A) comprises at least one liquid hydrocarbon-based fatty substance iii) chosen from:
plant oils formed by fatty acid esters of polyols, in particular triglycerides, such as sunflower oil, sesame oil, rapeseed oil, macadamia oil, soybean oil, sweet almond oil, beauty-leaf oil, palm oil, grapeseed oil, corn oil, arara oil, cottonseed oil, apricot oil, avocado oil, jojoba oil, olive oil or cereal germ oil;
linear, branched or cyclic esters containing more than 6 carbon atoms, notably 6 to 30 carbon atoms; and notably isononyl isononanoate;
and more particularly the esters of formula Rd—C(O)—O—Re in which Rd represents a higher fatty acid residue including from 7 to 19 carbon atoms and Re represents a hydrocarbon-based chain including from 3 to 20 carbon atoms, such as palmitates, adipates, myristates and benzoates, notably diisopropyl adipate and isopropyl myristate;
hydrocarbons and notably volatile or non-volatile, linear, branched and/or cyclic alkanes, such as C5-C60 isoparaffins, which are optionally volatile, such as isododecane, Parleam (hydrogenated polyisobutene), isohexadecane, cyclohexane or Isopars; or else liquid paraffins, liquid petroleum jelly, or hydrogenated polyisobutylene; notably isododecane; ethers containing 6 to 30 carbon atoms;
aliphatic fatty monoalcohols containing 6 to 30 carbon atoms, the hydrocarbon-based chain not including any substitution groups, such as oleyl alcohol, decanol, dodecanol, octadecanol, octyldodecanol and linoleyl alcohol; notably octyldodecanol;
polyols containing 6 to 30 carbon atoms, such as hexylene glycol; and
their mixtures, such as the combination of undecane and tridecane such as for example CETIOL UT®, preferably isododecane, or mixtures of linear or branched C8-C10 fatty acid esters and C12 fatty alcohol -C18 and alkanes resulting from the hydrogenation / complete reduction of mixtures of fatty acids obtained from Cocos Nucifera oil (coconut), in particular dodecane such as mixtures of cococaprylate / caprate and dodecane, there may be mentioned those of the INCI name coconut alkanes (and) Coco-caprylate / caprate marketed under the name VEGELIGHT 1212LC® by Grant Industries; or mixtures of C9-C12 alkanes, the chains of which comprise 9 to 12 carbon atoms, preferably linear or branched, C9-C12 alkanes, in particular comprising dodecane, there may be mentioned the mixture of oil from INCI name C9-12 ALKANE, VEGELIGHT SILK® marketed by BioSynthls.
their mixtures..

Advantageously, the hydrocarbon-based liquid fatty substance(s) of the invention are apolar, i.e. formed solely of carbon and hydrogen atoms.

Preferably, the dispersion (A) comprises at least one apolar liquid hydrocarbon-based fatty substance iii) preferably chosen from:
linear or branched C8-C30, in particular C10-C20 and more particularly C10-C16 alkanes, which are volatile or non-volatile, preferably volatile;
non-aromatic cyclic C5-C12 alkanes, which are volatile or non-volatile, preferably volatile; and
mixtures thereof.

The liquid hydrocarbon-based fatty substance(s) are preferably chosen from hydrocarbon-based oils containing from 8 to 16 carbon atoms, in particular containing from 10 to 14 carbon atoms, which are preferably volatile, more particularly the apolar oils, described previously.

Among the branched C8-C16 and notably C10-C14 alkanes that are suitable for use as liquid hydrocarbon-based fatty substances iii) in the dispersion of the invention, mention may be made of:
isoalkanes of petroleum origin (also known as isoparaffins), such as isododecane (also known as 2,2,4,4,6-pentamethylheptane), isodecane and, for example, the oils sold under the Isopar or Permethyl trade names,
linear alkanes, for instance n-dodecane (C12) and n-tetradecane (C14) sold by Sasol under the references, respectively, Parafol 12-97 and Parafol 14-97, and also mixtures thereof, the undecane-tridecane mixture, mixtures of n-undecane (C11) and of n-tridecane (C13) from the company Cognis, and mixtures thereof.

Preferentially, the liquid hydrocarbon-based fatty substance(s) iii) of the invention are apolar, more particularly isododecane.

According to another advantageous embodiment of the invention, the hydrocarbon-based liquid fatty substance(s) are a mixture of non-volatile and volatile oil; preferably, the mixture comprises isododecane as volatile oil or a mixture of oils, notably of undecane and tridecane or else isononyl isononanoate or octyldodecanol.

Preferably, the liquid hydrocarbon-based fatty substance(s) iii) are present in the dispersion of the invention in an amount of between 1 % and 25% by weight, more preferentially between 2.5% and 20% by weight, even more preferentially between 4% and 15% and preferentially between 10% and 11.2% by weight relative to the total weight of said dispersion (A) with water v).

According to a particular embodiment of the invention, the liquid hydrocarbon-based fatty substance(s) iii) are present in the dispersion of the invention in an amount of between 1.5% and 30% by weight, more preferentially between 4% and 20% by weight, even more preferentially between 8% and 15% and notably between 12% and 13% by weight relative to the total weight of said dispersion (A) free of water.

According to a particular embodiment of the invention, the one or more volatile hydrocarbon is present from about 1 to about 85 wt.% based on the total weight of the skin tightening composition.

In some instances, the skin tightening composition includes at least one or more volatile hydrocarbon chosen from isoparaffin, isohexadecane, isododecane, isodecane, undecane, tridecane, dodecane, isohexyl, isodecyl, neopentanoate, or a combination thereof. In at least one instance, isododecane and/or isoparaffins (e.g., C₈-₉ isoparaffin) are preferred. The skin tightening composition may be formulated to include volatile hydrocarbons that contain no silicon atoms.

The total amount of the volatile hydrocarbon may vary but is typically about 1 to about 85 wt.% based on the total weight of the skin tightening composition. In some cases, the total amount of volatile hydrocarbons is about 5 to about 85 wt%, about 10 to about 85 wt%, about 12 to about 85 wt%, about 14 to about 85 wt%, about 15 to about 85 wt%, about 1 to about 80 wt. %, about 5 to about 80 wt. %, about 10 to about 80 wt. %, about 15 to about 80 wt. %, about 10 to about 85 wt. %, about 20 to about 85 wt.%, about 20 to about 80 wt.%, about 30 to about 85 wt.%, based on the total weight of the skin tightening composition. In some cases, the total amount of volatile hydrocarbons is about 30 to about 80 wt. %, about 30 to about 75 wt.%; about 35 to about 85 wt. %, about 35 to about 80 wt. %, about 35 to about 75 wt.%; about 40 to about 85 wt. %, about 40 to about 80 wt. %, about 40 to about 75 wt.%; about 45 to about 85 wt. %, about 45 to about 80 wt. %, about 45 to about 75 wt.%; about 50 to about 85 wt. %, about 50 to about 80 wt. %, about 50 to about 75 wt.%; about 55 to about 85 wt. %, about 55 to about 80 wt. %, about 55 to about 75 wt.%; about 60 to about 85 wt. %, about 60 to about 80 wt. %, about 60 to about 75 wt.%; about 65 to about 85 wt. %, about 65 to about 80 wt.%, or about 65 to about 75 wt.%, including ranges and subranges thereof, based on the total weight of the skin tightening composition.

According to a particular embodiment of the invention, the weight ratio of the sum of the ingredients [i) + ii)] / iii) is less than or equal to 3, more particularly [i) + ii)] / iii) the mass ratio is between 1 and 2.5, even more particularly between 1.5 and 2.4, preferentially between 1.7 and 2.2.

### The Polyol(s) IV

The dispersion (A) of the instant disclosure also comprises one or more polyols that are liquid at 20° C. and 1 atm.

The term “polyol” means a compound which is liquid at 20° C. and 1 atm, comprising at least 2 hydroxyl groups, preferably between 2 and 10 hydroxyl groups (OH), and comprising at least one carbon atom. Particularly, the polyol(s) of the invention are chosen from compounds comprising at least 2 OH groups, preferably between 2 and 8 OH groups, more preferentially 2 or 3 OH groups, even more preferentially 3 OH groups, and comprising a linear or branched, cyclic or acyclic, saturated or unsaturated hydrocarbon-based chain comprising from 1 to 10 carbon atoms, in particular between 2 and 8 carbon atoms.

More particularly, the polyol(s) of the instant disclosure have a molecular weight of between 50 g/mol and 300 g/mol, more particularly between 80 g/mol and 150 g/mol.

According to a particular embodiment of the instant disclosure, the polyol(s) iv) are chosen from:
hydrocarbon-based (C2-C6)alkanediol compounds and
(C2-C6)alkanetriol such as glycerol and butanetriols, and pentanetriols, and hexanetriols such as hexane-1,2,6-triol.

Preferentially, the polyol(s) iv) are chosen from (C2-C6)alkanetriol hydrocarbon-based compounds such as glycerol.

According to a particular embodiment of the instant disclosure, the dispersion (A) comprises one or more polyols in an amount of greater than or equal to 5% by weight and less than 95% by weight relative to the total weight of the dispersion free of water; in particular, the amount of polyol(s) in the dispersion is between 10% and 90% by weight relative to the total weight of the dispersion free of water, more particularly between 25% and 90% by weight, more particularly between 35% and 90% by weight, relative to the total weight of the dispersion (A) free of water; and more preferentially between 50% and 70%.

According to another particular embodiment of the instant disclosure, the dispersion (A) comprises one or more polyols in an amount of greater than or equal to 15% by weight and less than 90% by weight relative to the total weight of the dispersion with water; in particular, the amount of polyol(s) in the dispersion is between 20% and 85% by weight relative to the total weight of the dispersion with water, more particularly between 25% and 80% by weight, more particularly between 40% and 60% by weight, relative to the total weight of the dispersion (A) with water.

In some instances, the skin tightening composition includes one or more polyols, in addition to the one or more polyol(s) from A)iv), which may be the same or different than the one from A)iv).

According to a particular embodiment of the invention, the one or more polyols is present from about 1 to about 50 wt.% based on the total weight of the skin tightening composition.

The total amount of the polyols may vary but is typically about 1 to about 50 wt.% based on the total weight of the skin tightening composition. In some cases, the total amount of polyols is from about 1, 2, 3, 4, 5, 6, 7, 8, 9, 10,12,14,16,18, 20, 22, 24 to about 24, 25, 26, 27, 28, 29, 30, 31, 32, 33, 34, 35, 36, 38, 40, 42, 44, 46, 48, and to 50 wt.% based on the total weight of the skin tightening composition.

In some embodiments of the skin tightening composition, the weight ratio of the total amount of i) of dispersion A to the total amount of the amount of polyols B is between around 1:1 to 4:1. In one or more embodiments of the skin tightening composition, the weight ratio of the total amount of i) of dispersion A to the total amount of the amount of polyols B is 1:1 to 4:1, 1:1 to 3.5:1, 1:1 to 3:1, 1:1 to 2.5:1, 1:1 to 2:1, 1:1 to 1.5:1, or any ranges and subranges thereof.

### Optionally Water V

According to a particular embodiment of the instant disclosure, the dispersion (A), notably the hydrogel, comprises water v) in an amount of between 5% and 40% by weight relative to the total weight of the dispersion, more particularly between 7% and 35% by weight and preferentially between 10.8% and 26.0% by weight relative to the total weight of the dispersion (A).

According to a particular embodiment of the instant disclosure, the weight ratio of the hydrocarbon-based liquid fatty substance(s) iii)/the water v) is between 0.1 and 5, more particularly between 0.2 and 1, preferably between 0.3 and 0.7, more preferentially between 0.4 and 0.6.

The water that is suitable for use in the instant disclosure may be tap water, distilled water, spring water, a floral water such as cornflower water and/or a mineral water such as Vittel water, Lucas water or La Roche Posay water and/or a thermal water.

The dispersion (A) may also comprise one or more water-miscible solvents.

According to the instant disclosure, the term “water-miscible solvent” denotes a compound that is liquid at room temperature and water-miscible (miscibility with water of greater than 50% by weight at 25° C. and atmospheric pressure).

The water-miscible solvents that may be used in dispersion (A) of the instant disclosure may also be volatile.

Among the water-miscible solvents that may be used in the composition according to the invention, mention may notably be made of lower monoalcohols containing from 2 to 5 carbon atoms such as ethanol and isopropanol.

The water may also comprise any water-soluble or water-dispersible compound that is compatible with an aqueous phase, such as associated film-forming polymers, surfactants, and mixtures thereof.

The term “surfactant” means a “surface agent”, which is a compound that is capable of modifying the surface tension between two surfaces; surfactants are amphiphilic molecules, i.e. they contain two parts of different polarity, one lipophilic and apolar, and the other hydrophilic and polar. The surfactants may be nonionic, anionic, amphoteric or cationic active agents.

According to a preferred embodiment of the instant disclosure, the dispersion (A) of the instant disclosure does not comprise more than 3% by weight of surfactants relative to the total weight of the dispersion, preferentially not more than 2% by weight of surfactants relative to the total weight of the dispersion, more particularly not more than 1% by weight of surfactants relative to the total weight of the dispersion; even more preferentially, the composition does not comprise more than 0.5% by weight of surfactants relative to the total weight of the dispersion, and better still the mixture does not comprise any surfactant.

### Optionally One or More Cosmetic Active Agents VI

The skin tightening compositions may, optionally, include one or more skin active agents, such as anti-aging agent, anti-wrinkle actives, anti-depuffing agent, anti-dark circles actives, anti-oxidants, humectants, moisturizing ingredients, depigmenting agents, and/or agents for treating oily skin etc. The skin active agents may be included in the skin tightening composition in an amount ranging from greater than zero to about to about 10 wt.%, based on the total weight of the composition. For example, the total amount of skin active agents may be from greater than zero to about 9 wt.%, greater than zero to about 8 wt.%, greater than zero to about 7 wt.%, greater than zero to about 6 wt.%, greater than zero to about 5 wt.%, greater than zero to about 4 wt.%, greater than zero to about 3 wt.%, greater than zero to about 2 wt.%; about 10 ppm to about 10 wt.% (100,000 ppm), about 10 ppm to about 5 wt.% (50,000 ppm), about 10 ppm to about 2.5 wt.% (25,000 ppm), about 10 ppm to about 1 wt.% (10,000 ppm), about 10 ppm to about 0.5 wt.% (5,000 ppm), about 10 ppm to about 0.3 wt.% (3,000 ppm), about 10 ppm to about 0.2 wt.% (2,000 ppm), about 10 ppm to about 0.1 wt.% (1,000 ppm), about 10 ppm to 500 ppm; about 0.1 to about 10 wt.%, about 0.1 to about 5 wt.%, about 0.1 to about 2.5 wt.%, about 0.1 to about 1 wt.%, about 0.1 to about 0.5 wt.%; about 1 to about 10 wt.%, about 1 to about 8 wt.%, about 1 to about 6 wt.%, about 1 to about 5 wt.%, about 1 to about 4 wt.%, about 1 to about 3 wt.%; about 2 to about 10 wt.%, about 2 to about 8 wt.%, about 2 to about 6 wt.%, about 2 to about 5 wt.%, about 2 to about 4 wt.%; about 3 to about 10 wt.%, about 3 to about 8 wt.%, about 3 to about 6 wt.%, about 3 to about 5 wt.%; about 4 to about 10 wt.%, about 4 to about 8 wt.%, or about 4 to about 6 wt.%, including ranges and subranges there between, based on the total weight of the skin tightening composition.

According to a particular embodiment of the instant disclosure, the dispersion (A) of the invention comprises one or more cosmetic active agents vi) different from iii) and from iv), chosen from f) dyes, g) pigments; h) active agents for caring for keratin materials, and i) UV (A) and/or UV (B) screening agents, and also m) mixtures thereof.

According to a preferred embodiment of the instant disclosure, the cosmetic active agent(s) of the invention are chosen from g) pigments.

According to a particular embodiment of the instant disclosure, the cosmetic active agent(s) of the invention are chosen from h) active agents for caring for keratin materials, preferably skincare active agents, in particular antioxidants or moisturizers that are notably polar, different from iv).

The term “moisturizer” means a compound which fixes water, which enables the moisturization of keratin materials, making it possible to more efficiently prevent and/or combat the signs of drying-out of the skin. However, the term “moisturizer” does not cover substances which only have an emollient effect, such as petroleum jelly and plant oils or silicone oils, which are compounds that deposit an occlusive lipid film on the skin, preventing the evaporation of water.

In particular, the dispersion (A) comprises one or more moisturizers which are hygroscopic substances that bring about rehydration of the skin by capturing atmospheric water and by retaining water in keratin materials, notably the skin.

Moisturizers that may be mentioned include the constituents of NMF (Natural Moisturizing Factor) which are small water-soluble molecules with high hygroscopic power which play a moisturizing role.

NMF mainly consists of a) amino acids (preferably in a weight amount of between 50% and 80% such as 65%), b) organic acids (preferably in a weight amount of between 10% and 30% such as 21%) including pyrrolidonecarboxylic acid, c) ions (anions or cations, in a weight amount of between 3% and 10% such as 8%), d) urea (in a weight amount of between 1% and 6% such as 4%) and e) sugars (in a weight amount of between 1% and 4% such as 2%, it being understood that the percentages are relative to the total weight of a) + b) + c) + d) + e)).

Mention may be made in particular of organic acids and preferably (poly)hydroxylated carboxylic acids and salts thereof.

According to a particular embodiment of the invention, the ingredient(s) vi) are chosen from (poly)hydroxylated carboxylic acids. More particularly, the carboxylic acids are saturated or unsaturated, cyclic or acyclic hydrocarbon-based (poly)hydroxylated compounds comprising from 2 to 20 carbon atoms, including one or more carboxyl or carboxylate groups, and one or more hydroxyl groups, and optionally comprising one or more heteroatoms such as nitrogen.

Among the (poly)hydroxylated carboxylic acids, mention may notably be made of lactic acid and the lactate salt of an alkali metal such as sodium [72-17-3], optionally unsaturated (C1-C20) carboxylic acids such as linoleic acid [60-33-3], heteroarylcarboxylic acids such as pyrrolidonecarboxylic acid, notably 2-pyrrolidinone-5-carboxylic acid (PCA) [98-79-3],

The organic (poly)hydroxylated compound(s) that are solid at 20° C. and at 1 atm vi) are organic polyols different from the water-miscible polyols containing from 4 to 8 carbon atoms as defined previously.

According to another particular embodiment of the invention, the ingredient(s) vi) are chosen from amino acids, notably chosen from alanine, arginine, aspartic acid, citrulline, glutamic acid, urocanic acid, glycine, histidine, leucine, lysine, ornithine, phenylalanine, proline, serine, threonine, tryptophan, tyrosine, valine, salts thereof and mixtures thereof. Glycine and serine are preferred.

An example of a commercially available starting material comprising an amino acid mixture that may be used in the composition according to the invention is the product sold by Cognis under the trade name Hydrosmyl LS 4513, which notably comprises a mixture of arginine, serine, citrulline, alanine, threonine, glutamic acid and lysine hydrochloride.

As a variant, use may be made of acyl derivatives of amino acids, such as N-α-benzoyl-L-arginine.

According to another variant of the invention, the ingredient(s) vi) are chosen from glycosaminoglycans GAG and sulfates thereof. Mention may be made of: hyaluronic acid or hyaluronan (HA), heparan sulfate (HS), heparin (HP), chondroitin, chondroitin sulfate (CS), chondroitin 4-sulfate or chondroitin sulfate A (CSA), chondroitin 6-sulfate or chondroitin sulfate C (CSC), dermatan sulfate or chondroitin sulfate B (CSB) and keratan sulfate (KS), which differs from the other glycosaminoglycans by the presence of galactose in place of uronic acid, and dextran sulfate. Mention may also be made of chitosan. Hyaluronic acid and the sodium salt thereof are preferred.

Another category of moisturizers according to the invention consists of urea [57 13 6] and derivatives such as hydroxyalkyl ureas, notably of general formula (VII): [Chem. 7] R1R2N—C(O)—NR3R4 (VII) in which formula (VII) R₁, R₂, R₃ and R₄ each independently represent a hydrogen atom, a C₁-C₄ alkyl group or a C₂-C₆ hydroxyalkyl group which may contain from 1 to 5 hydroxyl groups, at least one of the radicals R₁-R₄ representing a hydroxyalkyl group, and the salts thereof, solvates thereof such as hydrates and optical isomers thereof.

Among the salts, mention may be made of the salts of mineral acids, such as sulfuric acid, hydrochloric acid, hydrobromic acid, hydriodic acid, phosphoric acid and boric acid. Mention may also be made of the salts of organic acids, which may include one or more carboxylic, sulfonic or phosphonic acid groups. They may be linear, branched or cyclic aliphatic acids, or alternatively aromatic acids. These acids may also include one or more heteroatoms chosen from O and N, for example in the form of hydroxyl groups. Mention may notably be made of propionic acid, acetic acid, terephthalic acid, citric acid and tartaric acid.

The term “solvate” means a stoichiometric mixture of said compound in question with one or more molecules of water or of organic solvent, such a mixture being derived from the synthesis of said compound.

According to a particular embodiment of the invention, the ingredient(s) vi) chosen from urea and hydroxyalkyl ureas notably of formula (VII) according to the invention notably make it possible to plasticize the network formed by the glycosaminoglycans and thus to reinforce the moisturization that they afford to the keratin materials.

More particularly, the ingredient(s) vi) in the present invention are chosen from urea; trehalose; ectoin; a phospholipid such as lecithin, or a polymer containing phosphorylcholine groups, such as poly-2-(methacryloyloxyethyl)phosphorylcholine sold under the trade name Lipidure HM by NOF Corporation; cholesterol; phytosterols such as sitosterol, campesterol, stigmasterol, cafesterol and ergosterol; essential fatty acids such as oleic acid, linoleic acid and linolenic acid; cinnamic acid; an extract of Theobroma cacao, in particular an extract of Theobroma cacao bean pods, obtained, for example, by aqueous-alcoholic extraction, such as the product sold by the company Solabia under the name Caobromine® and γ-aminobutyric acid.

According to a particular embodiment of the invention, the ingredient(s) vi) are chosen from dehydroepiandrosterone (DHEA) and the 7-oxide and 17-alkyl derivatives thereof, as described in patent applications EP-1 366 743 and EP-1 413 291, respectively, the content of which is incorporated herein by reference. 7-Hydroxy DHEA, 3-O-acetyl 7 benzoyloxy DHEA and 5-cholesten-3β,25-diol-7-one are preferred for use in the present invention.

According to another particular embodiment of the invention, the ingredient(s) vi) are chosen from the alkyl esters of (dihydro)jasmonic acid, and in particular methyl dihydrojasmonate, described in patent application EP-1 442 737, the content of which is incorporated herein by reference.

Moisturizers that may also be used include compounds which increase keratinocyte differentiation, chosen, for example, from: a peptide extract of lupin, such as the product sold by the company Silab under the trade name Structurine; sodium beta-sitosteryl sulfate, for example sold by the company Seporga under the trade name Phytocohesine; water-soluble extracts of corn, such as the product sold by the company Solabia under the trade name Phytovityl; niacinamide; a peptide extract of Voandzeia subterranea such as the product sold by the company Laboratoires Serobiologiques under the trade name Filladyn LS 9397.

According to a particular embodiment of the invention, the ingredient(s) vi) are chosen from heterogeneous polyholosides, polyols such as propylene glycol, glycerin, glycerol [56-81-5], sorbitol [50-70-4], ethoxylated polyols, esterified polyols and glycosaminoglycans (GAG) notably from soybean protein [68607-88-5], sulfated GAGs and hyaluronic acid or mucopolysaccharides, and hydrolysed proteins of hydrolysed soybean GAG, tocopheryl acetate [7695-91-2].

According to yet another particular embodiment of the present patent application, the cosmetic active agent(s) vi) of the invention are chosen from i) antioxidants.

The term “antioxidant” or AOX means a natural or synthetic compound which reduces, slows down or prevents the oxidation of other chemical substances with which it is in contact (see, for example, Antioxidants, C. Kröhnke et al. Ullmann’s Encyclopedia of Industrial Chemistry Wiley-VCH Verlag GmbH & Co. KGaA, Weinheim 10.1002/14356007.a03_091.pub2, ISBN: 9783527306732| DOI: 10.1002/14356007 (2015)).

According to a particular embodiment of the invention, the antioxidant(s) are chosen from the compounds of formulae (AOX-1), (AOX-2) and (AOX-3) below and also the organic or mineral acid or base salts thereof, the optical, geometric and tautomeric isomers thereof, and solvates thereof such as hydrates:

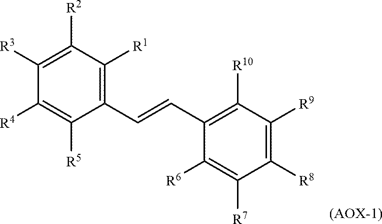

in which formula (AOX-1):
R¹ to R¹⁰ represent a hydrogen atom or a group chosen from hydroxyl, (di)(C₁-C₄)(alkyl)amino, (C₁-C₄)alkyl, (C₁-C₄)alkoxy, (C₁-C₄)alkylthio, —(Xₐ)ₚ—C(X_{b})—(X_{c})_{q}—R¹¹, -oside and -O-oside, such as glucopyranoside, with p and q equal to 0 or 1, Xₐ, X_{b} and X_{c}, which may be identical or different, represent O, S or NR¹², preferably O, and R¹¹ and R¹², which may be identical or different, represent a hydrogen atom, a (C₁-C₄)alkyl group or phenyl, it being understood that at least one of the radicals R¹ to R¹⁰ represents a hydroxyl group,
preferably, R¹, R³, R⁵, R⁶, R⁷, R⁹ and R¹⁰ represent a hydrogen atom or a (C₁-C₄)alkyl group, more preferentially a hydrogen atom.

Preferably, R², R⁴ and R⁸ represent a hydroxyl group, (C₁-C₄)alkoxy or -O-oside such as O-glucopyranoside;

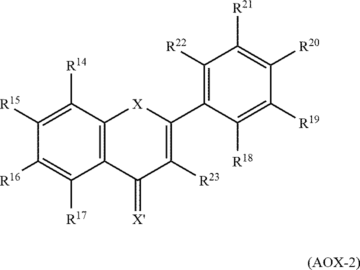

in which formula (AOX-2):
X and X′ independently denote O or S;
R¹⁴ to R²³ represent a hydrogen atom or a group chosen from hydroxyl, (di)(C₁-C₄)(alkyl)amino, (C₁-C₄)alkoxy, (C₁-C₄)alkylthio, —(Xₐ)ₚ—C(X_{b})—(X_{c})_{q}—R²⁴, -oside and -O-oside, such as glucuronopyranoside, with p and q equal to 0 or 1, Xₐ, X_{b} or X_{c}, which may be identical or different, represent O, S or NR²⁵, preferably O, and R²⁴ and R²⁵, which may be identical or different, represent a hydrogen atom, a (C₁-C₄)alkyl group or phenyl, it being understood that at least one of the radicals R¹⁴ to R²³ represents a hydroxyl group.

Preferably, R¹⁴, R¹⁸, R¹⁹, R²⁰, R²¹ and R²² represent a hydrogen atom or a (C₁-C₄)alkyl group, more preferentially a hydrogen atom.

Preferably, R¹⁵ represents a hydroxyl group, (C₁-C₄)alkoxy or -O-oside such as O-glucuronopyranoside.

Preferably, X and X′ are identical and denote an oxygen atom.

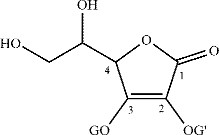

(AOX-3) in which formula (AOX-3):
G and G′, which may be identical or different, represent a hydrogen atom, an alkali metal such as Na⁺, an ammonium, an alkaline-earth metal such as Mg²⁺, or a group chosen from: i) (C₁-C₆)alkyl such as methyl or ethyl, ii) benzyl, iii) —C(X_{b})—(X_{c})_{q}—R²⁴; iv) —P(O)(O^{—})₂ M₂, v) —P(O)(O^{—})₂ M′, vi) -oside such as glucopyranoside or xylose, and with q, X_{b} and X_{c} as defined previously, M representing a hydrogen atom or an alkali metal such as Na⁺, or ammonium; and M′ represents an alkaline-earth metal such as Mg²⁺.

Preferably, G is chosen from hydrogen, an alkali metal such as sodium, a (C₁-C₄)alkyl group such as ethyl, and P(O)(O⁻)₂ M′ with M′ representing an alkaline-earth metal such as Mg²⁺.

According to a particular embodiment of the invention, G′ represents a hydrogen atom, an alkali metal such as sodium, a (C₁-C₄)alkyl group such as ethyl; —P(O)(O^{—})₂ M₂, —P(O)(O^{—})₂ M′, and -oside such as glucopyranoside or xylose. Preferably, the carbon atom in position 4 is of R configuration.

More particularly, the oside group is chosen from

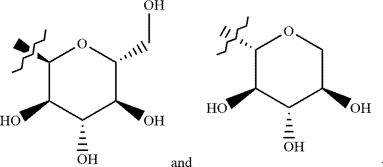

According to a particular embodiment of the invention, the antioxidant(s) are chosen from AOX-3, preferentially chosen from vitamin C (or ascorbic acid), iso-vitamin C (or erythorbic acid), ascorbyl phosphates of alkaline-earth metals, notably of magnesium, 3-O-ethylascorbic acid, ascorbyl glucoside and ascorbyl xyloside with the xylose group in position 2 or 3.

According to a particular embodiment of the invention, the antioxidant(s) are chosen from i) vitamin C (ascorbic acid), ii) resveratrol and also the glycosyl forms thereof such as polydatin, or the methoxyl forms thereof such as 3′,5′-dimethoxyresveratrol, and iii) baicalin.

Preferentially, the antioxidant(s) (a) of the invention are chosen from AOX-3, notably vitamin C.

The weight amount of antioxidant(s) is inclusively between 0.1% and 30%, in particular between 0.2% and 20%, more particularly between 0.4% and 15%, preferentially between 0.6% and 10%, such as 7% by weight relative to the total weight of the composition.

According to yet another particular embodiment of the present patent application, the cosmetic active agent(s) of the invention are chosen from i) UV(A) and/or UV(B) screening agents, and a mixture thereof.

According to a particular embodiment of the invention, the dispersion (A) comprises iv) one or more cosmetic active agents chosen from pigments.

The pigment(s) more particularly represent from 0.5% to 40% by weight and preferably from 1% to 20% by weight relative to the total weight of the dispersion (A).

The pigments are white or coloured solid particles which are naturally insoluble in the hydrophilic and lipophilic liquid phases usually employed in cosmetics or which are rendered insoluble by formulation in the form of a lake, where appropriate. More particularly, the pigments have little or no solubility in aqueous-alcoholic media.

The pigments that may be used are notably chosen from the organic and/or mineral pigments known in the art, notably those described in Kirk-Othmer’s Encyclopedia of Chemical Technology and in Ullmann’s Encyclopedia of Industrial Chemistry. Pigments that may notably be mentioned include organic and mineral pigments such as those defined and described in Ullmann’s Encyclopedia of Industrial Chemistry “Pigments, Organic”, 2005 Wiley-VCH Verlag GmbH & Co. KGaA, Weinheim 10.1002/14356007.a20 371 and ibid, “Pigments, Inorganic, 1. General” 2009 Wiley-VCH Verlag GmbH & Co. KGaA, Weinheim10.1002/14356007.a20_243.pub3.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann’s encyclopaedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment.

The term “organic pigment” refers to any pigment that satisfies the definition in Ullmann’s encyclopaedia in the chapter on organic pigments.

The organic pigment may notably be chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane and quinophthalone compounds.

In particular, the white or coloured organic pigments may be chosen from carmine, carbon black, aniline black, azo yellow, quinacridone, phthalocyanine blue, the blue pigments codified in the Colour Index under the references Cl 42090, 69800, 69825, 74100, 74160, the yellow pigments codified in the Colour Index under the references Cl 11680, 11710, 19140, 20040, 21100, 21108, 47000, 47005, the green pigments codified in the Colour Index under the references Cl 61565, 61570, 74260, the orange pigments codified in the Colour Index under the references Cl 11725, 45370, 71105, the red pigments codified in the Colour Index under the references Cl 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names: Cosmenyl Yellow IOG: Yellow 3 pigment (Cl 11710); Cosmenyl G yellow: Yellow 1 pigment (Cl 11680); Cosmenyl GR orange: Orange 43 pigment (Cl 71105); Cosmenyl R red: Red 4 pigment (Cl 12085); Cosmenyl FB carmine: Red 5 pigment (Cl 12490); Cosmenyl RL violet: Violet 23 pigment (Cl 51319); Cosmenyl A2R blue: Blue 15.1 pigment (Cl 74160); Cosmenyl GG green: Green 7 pigment (Cl 74260); Cosmenyl R black: Black 7 pigment (Cl 77266).

The pigments in accordance with the invention may also be in the form of composite pigments, as described in patent EP 1 184 426. These composite pigments may be composed notably of particles including a mineral core, at least one binder for attaching the organic pigments to the core, and at least one organic pigment which at least partially covers the core.

The organic pigment may also be a lake. The term “lake” means dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Among the dyes, mention may be made of carminic acid. Mention may also be made of the dyes known under the following names: D&C Red 21 (Cl 45 380), D&C Orange 5 (Cl 45 370), D&C Red 27 (Cl 45 410), D&C Orange 10 (Cl 45 425), D&C Red 3 (Cl 45 430), D&C Red 4 (Cl 15 510), D&C Red 33 (Cl 17 200), D&C Yellow 5 (Cl 19 140), D&C Yellow 6 (Cl 15 985), D&C Green 5 (Cl 61 570), D&C Yellow 10 (Cl 77 002), D&C Green 3 (Cl 42 053), D&C Blue 1 (Cl 42 090).

An example of a lake that may be mentioned is the product known under the following name: D&C Red 7 (Cl 15 850:1).

The pigment may also be a pigment with special effects. The term “pigments with special effects” means pigments that generally create a coloured appearance (characterized by a certain shade, a certain vivacity and a certain level of luminance) that is non-uniform and that changes as a function of the conditions of observation (light, temperature, angles of observation, etc.). They thereby differ from coloured pigments, which afford a standard uniform opaque, semi-transparent or transparent shade.

Several types of pigments with special effects exist: those with a low refractive index, such as fluorescent or photochromic pigments, and those with a higher refractive index, such as nacres, interference pigments or glitter flakes.

Examples of pigments with special effects that may be mentioned include nacreous pigments such as mica covered with titanium or with bismuth oxychloride, coloured nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. Nacreous pigments that may be mentioned include the nacres Cellini sold by BASF (mica-TiO2-lake), Prestige sold by Eckart (mica-TiO2), Prestige Bronze sold by Eckart (mica-Fe2O3) and Colorona sold by Merck (mica-TiO2-Fe2O3).

Mention may also be made of the gold-coloured nacres sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by the company Merck under the name Bronze fine (17384) (Colorona) and Bronze (17353) (Colorona) and by the company BASF under the name Super bronze (Cloisonne); the orange nacres sold notably by the company BASF under the name Orange 363C (Cloisonne) and Orange MCR 101 (Cosmica) and by the company Merck under the name Passion orange (Colorona) and Matte orange (17449) (Microna); the brown nacres sold notably by the company BASF under the name Nu-antique copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan opale G005 (Gemtone); the black nacres with a gold tint sold notably by the company BASF under the name Nu antique bronze 240 AB (Timica), the blue nacres sold notably by the company Merck under the name Matte blue (17433) (Microna), the white nacres with a silvery tint sold notably by the company Merck under the name Xirona Silver, and the golden-green pink-orange nacres sold notably by the company Merck under the name Indian summer (Xirona), and mixtures thereof.

Still as examples of nacres, mention may also be made of particles including a borosilicate substrate coated with titanium oxide.

Particles comprising a glass substrate coated with titanium oxide are notably sold under the name Metashine MC1080RY by the company Toyal.

Finally, examples of nacres that may also be mentioned include polyethylene terephthalate glitter flakes, notably those sold by the company Meadowbrook Inventions under the name Silver 1P 0.004X0.004 (silver glitter flakes). It is also possible to envisage multilayer pigments based on synthetic substrates, such as alumina, silica, calcium sodium borosilicate, calcium aluminium borosilicate and aluminium.

The pigments with special effects may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. more luminous points that contrast with their environment, making them appear to sparkle.

The reflective particles may be selected so as not to significantly alter the colouring effect generated by the colouring agents with which they are combined, and more particularly so as to optimize this effect in terms of colour rendition. They may more particularly have a yellow, pink, red, bronze, orange, brown, gold and/or coppery colour or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or mineral materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in JP-A-09188830, JP-A-10158450, JP-A-10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the names Crystal Star GF 550 and GF 2525 by this same company.

Use may also be made of particles comprising a metal substrate, such as silver, aluminium, iron, chromium, nickel, molybdenum, gold, copper, zinc, tin, magnesium, steel, bronze or titanium, said substrate being coated with at least one layer of at least one metal oxide, such as titanium oxide, aluminium oxide, iron oxide, cerium oxide, chromium oxide, silicon oxides and mixtures thereof.

Examples that may be mentioned include aluminium powder, bronze powder or copper powder coated with SiO2 sold under the name Visionaire by the company Eckart.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the composition according to the present invention is generally between 10 nm and 200 µm, preferably between 20 nm and 80 µm and more preferentially between 30 nm and 50 µm.

The pigments may be dispersed in the composition by means of a dispersant.

The dispersant serves to protect the dispersed particles against their agglomeration or flocculation. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of C8 to C20 fatty acid and of polyols such as glycerol or diglycerol are used, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments surface-treated beforehand that are useful in the context of the invention are pigments which have been completely or partially subjected to a surface treatment of chemical, electronic, electrochemical, mechanochemical or mechanical nature with an organic agent, such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polyethylene; (meth)acrylic polymers, for example polymethyl methacrylates; polymers and copolymers containing acrylate units; alkanolamines; silicone compounds, for example silicones, notably polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent US 4 578 266.

An organic agent covalently bonded to the pigments will preferably be used.

The agent for the surface treatment may represent from 0.1 % to 50% by weight relative to the total weight of the surface-treated pigment, preferably from 0.5% to 30% by weight and even more preferentially from 1% to 20% by weight relative to the total weight of the surface-treated pigment.

Preferably, the surface treatments of the pigments are chosen from the following treatments:
a PEG-silicone treatment, for instance the AQ surface treatment sold by LCW;
a methicone treatment, for instance the Sl surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a magnesium myristate treatment, for instance the MM surface treatment sold by LCW;
an aluminium dimyristate treatment, such as the Ml surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF + ITT surface treatment sold by Daito.

According to a particular embodiment of the invention, the dispersant is present with organic or mineral pigments in submicron-sized particulate form in the dye composition.

According to one embodiment, the dispersant and the pigment(s) are present in an amount (dispersant:pigment) of between 1:4 and 4:1, particularly between 1.5:3.5 and 3.5:1 or better still between 1.75:3 and 3:1.

The dispersant(s) may thus have a silicone backbone, such as silicone polyether and dispersants of amino silicone type other than the alkoxysilanes described previously. Among the suitable dispersants that may be mentioned are:
amino silicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxyl groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of amino silicone type other than the alkoxysilanes described previously and are cationic.

Preferably, the pigment(s) are chosen from mineral, mixed mineral-organic or organic pigments.

In one variant of the invention, the pigment(s) according to the invention are organic pigments, preferentially organic pigments surface-treated with an organic agent chosen from silicone compounds. In another variant of the invention, the pigment(s) according to the invention are mineral pigments.

The dispersion (A) may comprise one or more dyes f), in particular one or more direct dyes.

The term “direct dye” means natural and/or synthetic dyes, other than oxidation dyes. These are dyes that will spread superficially on the fibre.

They may be ionic or nonionic, preferably cationic or nonionic.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures; more preferentially red iron oxides.

According to a particular embodiment of the invention, the amount of pigments ranges from 0.5% to 40% and preferably from 1% to 20% relative to the weight of the dispersion (A) comprising them.

According to a particular embodiment of the invention, the skin tightening compositions may include one or more pigments, in addition to the one or more pigments from A)vi)g, which may be the same of different than the one from A)vi)g.

According to a particular embodiment of the invention, the one or more pigments is present from about 1 to about 20 wt.% based on the total weight of the skin tightening composition. In some other embodiments of the instant disclosure, the one or more pigments is present from about 1 to about 15 wt.% based on the total weight of the skin tightening composition.

The total amount of pigments present, may vary but is typically about 1.0 to about 40 wt.% based on the total weight of the skin tightening composition. The total amount of water dispersible pigments is typically from about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20, 20.5 to about 20.5, 21, 21.5, 22, 22.5, 23, 23.5, 24, 24.5, 25, 26, 28, 30, 32, 34, 36, 38, and wt.%, including ranges and subranges thereof, based on the total weight of the skin tightening composition.

### Optionally One or More Crosslinking Agents VII

According to a particular embodiment of the invention, the dispersion (A) of the invention comprises vii) one or more crosslinking agents.

According to a particular embodiment of the invention, the crosslinking agent(s) are chosen from the monomers of formulae (VI) and (VII) below:

TABLE 1

| | |
|---|---|
| 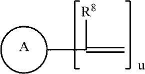 | 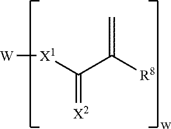 |
| (VI) | (VII) |

in which formulae (VI) and (VII):
· A represents an optionally substituted (hetero)aryl or optionally substituted (hetero)cycloalkyl group; preferably, A represents a phenyl group;
· R⁸, which may be identical or different, represents a hydrogen atom or a linear or branched, optionally substituted (C₁-C_{B})alkyl group, preferably a C₁-C₆ group such as methyl;
· X¹ and X², which may be identical or different, represent a heteroatom chosen from oxygen, sulfur and amino N(R″) with R″ being a hydrogen atom or a linear or branched (C₁-C₆)alkyl group; preferably, X¹ = O or NH, and X² = O; and more particularly X¹ = X² = an oxygen atom;
· W represents: i) either a group A as defined previously, in particular a 5- or 6-membered heteroaryl group such as pyridyl or a 5- to 8-membered heterocycloalkyl group comprising at least one oxygen atom such as tetrahydrofuryl, piperazinyl or hexahydrofuro[3,2-b]furyl, ii) or a group *—A—(CR⁹R¹⁰)ₓ—A—* when w is 2, with A as defined previously, R⁹ and R¹⁰, which may be identical or different, representing a hydrogen atom or a (C₁-C₆)alkyl group such as methyl; preferably, R⁹ and R¹⁰ = H, x represents an integer inclusively between 0 and 10, preferably x = 1 and * represents the point of attachment to the groups —X¹—C(X²)—C(═CH₂)—R⁸; iii) or a linear or branched, polyvalent, preferably divalent or trivalent, saturated or unsaturated, preferably saturated, hydrocarbon-based chain, which is optionally substituted, preferably with a hydroxyl group, optionally interrupted with one or more heteroatoms such as O, and comprising from 1 to 20 carbon atom
· p represents 0 or 1
· u and w represent an integer between 2 and 10 inclusive, and more particularly between 2 and 5, such that u = 2 and w = 2 or 3.

Preferably, the crosslinking agent(s) are of formula (VII), more particularly W represents a divalent C1-C10 alkyl group and w = 2 or a trivalent C1-C10 alkyl group and w = 3, said alkyl groups being interrupted with one or more heteroatoms such as O, more preferentially divalent C1-C8 alkyl and w = 2.

According to a preferred variant of the invention, the monomers of formulae (VI) and (VII) are chosen from the compounds from the table below, and also the optical and geometric isomers thereof, the tautomers thereof and the mineral or organic acid or base salts thereof:

TABLE 2

| | | |
|---|---|---|
| Name | Abbreviation | Structure |
| Styrene/divinylbenzene | (DVB) | 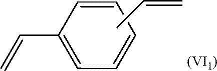 |
| Diisopropylbenzene | (DIB) | 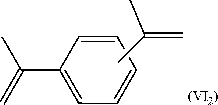 |
| 1,3-Phenylenediacrylamide; 1,4-Phenylenediacrylamide; | | 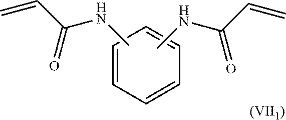 |
| N,N′-1,3-Phenylenebis(2-methyl-2-propenamide); N,N′-1,4-Phenylenebis(2-methyl-2-propenamide) | | 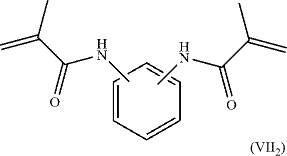 |
| 3,5-Bisacrylamidobenzoic acid with R″ identical and equal to H; and 3,5-bismethylacrylam idobenzoic acid with R″ identical and equal to CH₃ | | 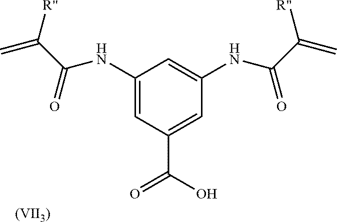 |
| | | R″ = H or (C₁-C₄)alkyl such as CH₃ |
| 2,6-Bisacryloylam idopyridine with R″ identical and equal to H; 2,6-Bismethylacryloylamidopyridin e with R″ identical and equal to CH₃ | | 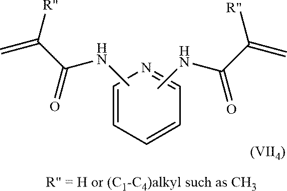 |
| 1,4-Diacryloylpiperazine with R″ identical and equal to H; 1,4-Dimethylacryloylpiperazine with R″ identical and equal to CH₃ | (DAP) | 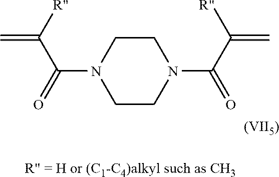 |
| Di Ethylene Glycol (n=2) Di Acrylate Diethylene Glycol Di Methacrylate (n = 2) n is an integer greater than or equal to 1 and preferably between 1 and 10 and preferably between 2 and 4 and preferably 2 | (DEGDA) (DEGDMA) | 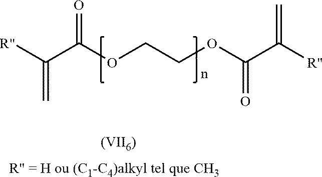 |
| Ethylene glycol dimethacrylate with R″ identical and equal to CH₃ (EGDMA); ethylene glycol diacrylate with R″ identical and equal to H | (EGDMA) | 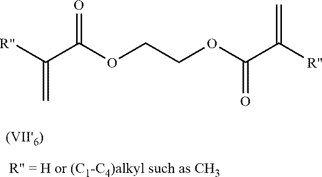 |
| Tetramethylene dimethacrylate with R″ identical and equal to CH₃; tetramethylene diacrylate with R″ identical and equal to H | (TDMA) | 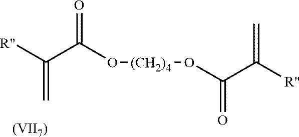 R″ = H or (C₁-C₄)alkyl such as CH₃ |
| Hexamethylene dimethacrylate with R″ identical and equal to CH₃ and hexamethylene diacrylate with R″ identical and equal to H | | 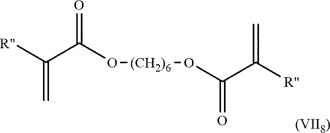 R″ = H or (C₁-C₄)alkyl such as CH₃ |
| Anhydroerythritol dimethacrylate with R″ identical and equal to CH₃ and anhydroerythritol diacrylate with R″ identical and equal to H | | 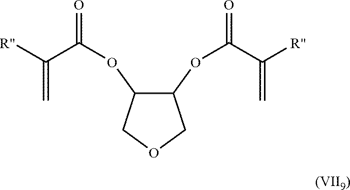 R″ = H or (C₁-C₄)alkyl such as CH₃ |
| 1,4:3,6-Dianhydro-p-sorbitol-2,5-dimethacrylate with R″ identical and equal to CH₃; 1,4-3,6-dianhydro-p-sorbitol-2,5-diacrylate with R″ identical and equal to H | | 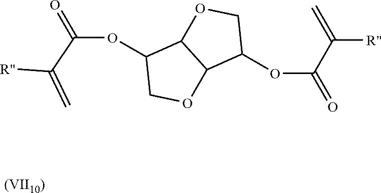 R″ = H or (C₁-C₄)alkyl such as CH₃ |
| Isopropenebis(1,4-phenylene) dimethacrylate with R″ identical and equal to CH₃; isopropenebis(1,4-phenylene) diacrylate with R″ identical and equal to H | | 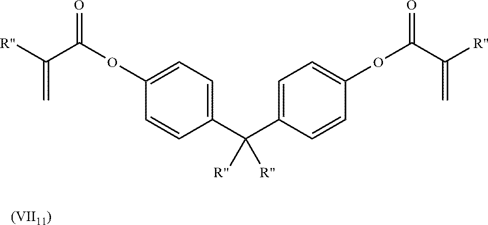 R″ = H or (C₁-C₄)alkyl such as CH₃ |
| 2,2-bis(Hydroxymethyl)butanol trimethacrylate with R″ identical and equal to CH₃ (TRIM); 2,2-bis(hydroxymethyl)butanol triacrylate with R″ identical and equal to H | (TRIM) | 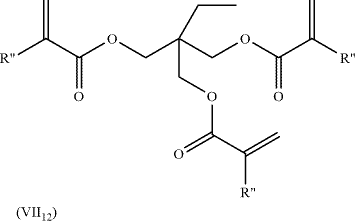 R″ = H or (C₁-C₄)alkyl such as CH₃ |
| Pentaerythrityl triacrylate with R″ identical and equal to H; Pentaerythrityl trimethacrylate with R″ identical and equal to CH₃ | | 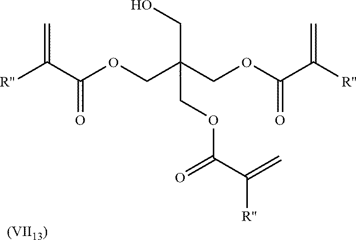 R″ = H or (C₁-C₄)alkyl such as CH₃ |
| Pentaerythrityl tetraacrylate with R″ identical and equal to H (PETRA); Pentaerythrityl tetramethacrylate with R″ identical and equal to CH₃ | (PETRA) | 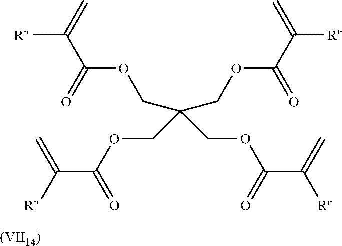 R″ = H or (C₁-C₄)alkyl such as CH₃ |
| N,O-Bismethacryloylethanolamine with R_{c} equal to ethylene and R″ identical and equal to CH₃ | (NOBE) | 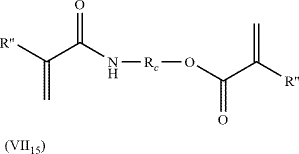 R_{c} = (C₁-C₆)alkylene such as ethylene R″ = H or (C₁-C₄)alkyl such as CH₃ |
| N, N′-Methylenebisacrylamide with R_{c} = CH₂ (MDAA); or N,N-1,2-Ethanediylbis(2-methyl-2-propenamide) N,N′-Ethylenebisacrylamide with R_{c} = CH2—CH2; N,N′-Butylenebisacrylamide with R_{c} = CH2—CH2—CH2—CH2; N,N′-Hexylenebisacrylamide with R_{c} = CH2—CH2—CH2—CH2—CH₂—CH₂ | (MDAA) | 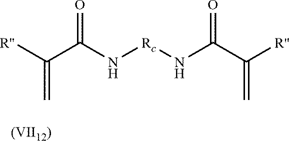 R_{c} = (C₁-C₆)alkylene R″ = H or (C₁-C₄)alkyl such as CH₃ |

In particular, the crosslinking agents of the invention are chosen from (VII₆), (VII₇) and (VII₈), such as ethylene glycol diacrylate; diethylene glycol diacrylate; tetra(ethylene glycol) diacrylate and diethylene glycol diacrylate (DEGDA), preferably DEGDA.

According to another particular embodiment of the invention, the crosslinking agent(s) are chosen from homopolymers or copolymers derived from the monomers of formulae (VI) and (VII) as defined previously; preferably the homopolymers and copolymers derived from the monomer of formula (VII), more preferentially with W representing a divalent C₁-C₁₀ alkyl group and w = 2 or a trivalent C₁-C₁₀ alkyl group and w = 3, said alkyl groups being interrupted with one or more heteroatoms such as O, more preferentially divalent C₁-C₈ alkyl and w = 2. In particular, the homopolymers and copolymers derived from monomers chosen from (VII₆) (VII₇) and (VII₈) such as polyethylene glycol) diacrylate, for instance Acrylate-PEG3500-Acrylate sold by Aldrich JKA4048 and Multi arm Poly(Ethylene Glycol) Acrylate such as 4 arm-PEG10K-Acrylate sold by Aldrich JKA7068; N,N′-methylenebisacrylamide.

Preferably, the crosslinking agent(s) of the invention are chosen from (VII₆), (VII₇) and (VII₈), such as ethylene glycol diacrylate; diethylene glycol diacrylate; tetra(ethylene glycol) diacrylate; poly(ethylene glycol) diacrylate, for instance acrylate-PEG3500-acrylate; multiarm poly(ethylene glycol) acrylate; and N,N′-methylenebisacrylamide; more preferentially ethylene glycol diacrylate.

### Optionally One or More Neutralizers VIII

According to one embodiment, the dispersion (A) or the composition (B) used in the preparation of the dispersion (A) comprises at least one neutralizer viii) in particular chosen from organic or inorganic alkaline agents , preferably inorganic, in particular the hydroxides of alkali or alkaline earth metals such as sodium or potassium hydroxide.

The term “neutralized” refers to any compound which can neutralize the pH to a pH value close to neutral pH, i.e. 7. To do this, use may be made of an organic or inorganic acid, or an alkaline agent (also known as a base), as defined below, notably alkali metal or alkaline-earth metal hydroxides such as sodium or potassium hydroxide or hydroxides of transition metals such as zinc.

More particularly, the neutralizer is added to composition (B) until the pH of said composition is obtained from 5 to 8, in particular 6.5 to 7.5 such as 7.

The mineral (or inorganic) alkaline agent(s) are preferably chosen from aqueous ammonia, alkali metal carbonates or bicarbonates such as sodium or potassium carbonate and sodium or potassium bicarbonate, sodium hydroxide or potassium hydroxide, or mixtures thereof.

According to an advantageous embodiment of the invention, the alkaline agent(s) are organic amines, i.e. they contain at least one substituted amine group (secondary or tertiary amine) or unsubstituted amine group (primary amine).

The organic alkaline agent(s) are more preferentially chosen from organic amines with a pKb at 25° C. of less than 12, preferably of less than 10 and even more advantageously of less than 6. If the alkaline agent(s) include several amine groups, it is the pKb corresponding to the function having the highest basicity.

Hybrid compounds that may be mentioned include the salts of the amines mentioned previously with acids as defined previously such as carbonic acid or hydrochloric acid.

The organic alkaline agent(s) are chosen, for example, from alkanolamines, oxyethylenated and/or oxypropylenated ethylenediamines, amino acids and the compounds of formula (VIII) below:

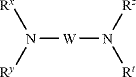

in which formula (VIII):
· W is a divalent C₁-C₆ alkylene radical optionally substituted with a hydroxyl group or a C₁-C₆ alkyl radical, and/or optionally interrupted with one or more heteroatoms such as oxygen or NR^{u};
· R^{X}, R^{y,} R^{z} R^{t} and R^{u}, which may be identical or different, represent a hydrogen

Examples of such amines that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C1-C8 alkyl groups bearing one or more hydroxyl radicals.

Alkanolamines such as monoalkanolamines, dialkanolamines or trialkanolamines comprising from one to three identical or different C1-C4 hydroxyalkyl radicals are in particular suitable for performing the invention.

Among compounds of this type, mention may be made of monoethanolamine, diethanolamine, triethanolamine, monoisopropanolamine, diisopropanolamine, N dimethylaminoethanolamine, 2-amino-2-methyl-1-propanol, triisopropanolamine, 2 amino-2-methyl-1,3-propanediol, 3-amino-1,2-propanediol, 3-dimethylamino-1,2-propanediol and tris(hydroxymethylamino)methane.

More particularly, the amino acids that may be used are of natural or synthetic origin, in their L, D or racemic form, and include at least one acid function chosen more particularly from carboxylic acid, sulfonic acid, phosphonic acid and phosphoric acid functions. The amino acids may be in neutral or ionic form. As amino acids that may be used in the present invention, mention may notably be made of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

More particularly, the neutralizer(s) of the invention are chosen from:
* alkaline agents notably chosen from mineral or inorganic alkaline agents such as LiOH, NaOH, KOH, Ca(OH)2, NH4OH or Zn(OH)2; and
* organic alkaline agents such as (C1-C8)alkylamines bearing primary, secondary or tertiary amine groups, notably trimethyl or butylamine, or (C1-C8)alkylamines bearing primary, secondary or tertiary groups including one or more nitrogen or oxygen atoms and/or including one or more hydroxyl groups; mention may notably be made of amino-2-methyl-2-propanol, triethanolamine and dimethylamino-2-propanol. Mention may also be made of lysine or 3-(dimethylamino)propylamine.

### Optionally One or More Radical Initiators IX

According to a particular embodiment of the invention, the process for preparing the dispersion (A) and the material (C) uses one or more radical initiators.

The term “radical initiators” in particular refers to free-radical initiators generated thermally or from light sources (see, for example, Macromol. Rapid Commun., Christian Decker, 23, 1067-1093 (2002); Encyclopedia of Polymer Science and Technology, “Photopolymerization, Free Radical”, http://onlinelibrary.wiley.com/doi/10.1002/0471440264.pst490/pdf; ibid, “Photopolymerization, Cationic”, http://onlinelibrary.wiley.com/doi/10.1002/0471440264.pst49⅟pdf; Macromol. Symp., 143, 45-63 (1999)). These photoactive compounds are not chemical oxidizing agents such as peroxides, including hydrogen peroxide or hydrogen peroxide-generating systems. Two major families may be distinguished:
the type I family, in which the photoactive compounds bring about, under irradiation, a unimolecular cleavage of the covalent bond to give a free-radical compound also symbolized by a “dot”, and
the type II family, in which the photoactive compounds, under irradiation, lead to a bimolecular reaction in which the excited state of the photoactive compounds interacts with a second molecule (or co-initiator) to generate free radicals.

More particularly, the radical initiators are chosen from the compounds of formulae (A), (B), (C), (D), (E) and (F), and also the organic or mineral acid salts thereof, the optical, geometrical or tautomeric isomers thereof and the solvates thereof such as the hydrates:

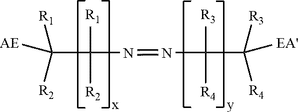

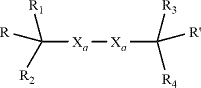

in which formula (A) or (B):
· R₁, R₂, R₃ and R₄, which may be identical or different, represent a hydrogen atom or a linear or branched (C₁-C₈)alkyl group which is optionally substituted, a linear or branched (C₁-C₈)alkoxy group or an optionally substituted aryl group, such as phenyl; or else R₁ and R₂ and/or R₃ and R₄ form, together with the carbon atoms which bear them, an optionally substituted 3- to 7-membered (hetero)cycloalkyl, in particular a (C₃-C₆)cycloalkyl, such as cyclohexyl; preferably, R₁, R₂, R₃ and R₄, which may be identical or different, represent a hydrogen atom or a linear or branched (C₁-C₆)alkyl group; x and y, which may be identical or different, represent an integer inclusively between 0 and 6 and preferably x and y =0;
· R and R′, which may be identical or different, preferably identical, represent i) a radical EW or EW′ as defined below, or a group chosen from ii) optionally substituted linear or branched (C₁-C₈)alkyl,iii) optionally substituted aryl or iv) optionally substituted aryl(C₁-C₈)alkyl; or else R with R¹ and/or R′ with R₃ form, together with the carbon atoms which bear them, a group C(X¹), with R₂ and R₄ being as defined previously, or R₂ and R₄, which may be identical or different, represent a group R₅-(X²)w⁻ in which w has the value 0 or 1, Rs represents a hydrogen atom, a linear or branched (C₁-C₈)alkyl group, an optionally substituted (hetero)aryl group, such as phenyl, or a (hetero)cycloalkyl group which is optionally substituted, notably with a (C₁-C₆)alkyl group, such as cyclohexyl optionally substituted with a (C₁-C₆)alkyl group, and X² is as defined below;
· EW and EW′, which may be identical or different, preferably identical, represent an electron-withdrawing group, which is preferably electron-withdrawing via a mesomeric effect -M, such as cyano, —C(X¹)—X²—Rₐ, phosph(on)ate, sulf(on)ate, nitro or nitroso; more particularly EW = EW′ = CN;
· Xₐ, which may be identical or different, represent a heteroatom chosen from oxygen and sulfur, a —C(O)—O— or —O—C(O)— group or a —O—C(O)—O— or —O—C(O)—O—group; preferably, Xₐ represent an oxygen atom;
X¹ and X², which may be identical or different, represent a heteroatom chosen from oxygen, sulfur and amino N(R″) with R″ being a hydrogen atom or a linear or branched (C₁-C₆)alkyl group; preferably, X¹ and X² represent an oxygen atom;

| | | | |
|---|---|---|---|
| 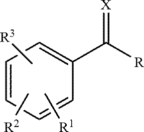 | 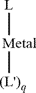 | 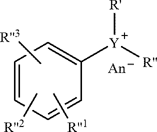 | 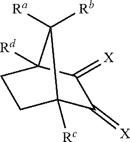 |
| (C) | (D) | (E) | (F) |

optionally substituted with a (C1-C6)alkyl group; in which formula (C), (D), (E) or (F):
· R represents a group chosen from:
   i) (C₁-C₁₀)alkyl, which is optionally substituted, preferably with one or more atoms or groups chosen from halogen, hydroxyl, (C₁-C₁₀)alkoxy, 5- to 10-membered (hetero)cycloalkyl such as morpholinyl, and amino RₐR_{b}N— with Rₐ and R_{b}, which may be identical or different, representing a hydrogen atom or a (C₁-C₁₀)alkyl group, or alternatively Rₐ and R_{b} form, together with the nitrogen atom which bears them, a heteroaryl or heterocycloalkyl group such as morpholino;
   ii) (C₁-C₁₀)alkoxy, which is optionally substituted, preferably with the same substituents as for i) (C₁-C₁₀)alkyl;
   iii) hydroxyl;
   iv) optionally substituted (hetero)aryl such as optionally substituted phenyl of formula 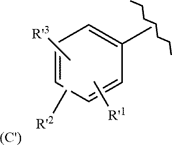
   with R′¹, R′² and R′³, which may be identical or different, being as defined for R¹, R² and R³ and representing the point of attachment to the rest of the molecule;
   v) (hetero)cycloalkyl which is optionally substituted, preferably with a hydroxyl group or a (C₁-C₁₀)alkyl group;
   vi) R⁴—(X)ₙ—C(X)—(X)_{n’}— with R⁴ representing an optionally substituted (C₁-C₁₀)alkyl group, an optionally substituted (hetero)aryl group such as optionally substituted phenyl of formula (C′), or an optionally substituted (hetero)cycloalkyl group, n and n′, which may be identical or different, being 0 or 1;
   vii) R_{c}R_{d}P(X)— with R_{c} representing an optionally substituted (C₁-C₁₀)alkyl group or an optionally substituted (hetero)aryl group, and R_{d} representing an optionally substituted (hetero)aryl group;
   viii) or else R¹ with R ortho to the group C(X)-R or R″ and R″¹ ortho to the group R′—Y+—R″ form, together with the atoms which bear them, a (hetero)cycle fused to the phenyl or (hetero)aryl fused to the phenyl, optionally substituted notably on the non-aromatic part with one or more oxo or thiooxo groups; preferably, R¹ with R ortho to the group C(X)-R form, together with the atoms which bear them and the fused phenyl ring, an anthraquinone group (G): 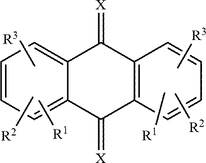
· R¹, R² or R³, which may be identical or different, represent i) a hydrogen atom, ii) a halogen atom such as chlorine, iii) an optionally substituted (C₁-C₁₀)alkyl, iv) (C₁-C₁₀)alkoxy which is optionally substituted notably with a hydroxyl group, v) optionally substituted (hetero)aryl, vi) optionally substituted (hetero)cycloalkyl, vii) carboxyl, viii) cyano, ix) nitro, x) nitroso, xi) —S(O)_{P}—OM with p being equal to 1 or 2, M representing a hydrogen atom, an alkali metal or alkaline-earth metal, xii) R⁴R⁵N-; xiii) R⁴—(X)ₙ—C(X)—(X)_{n’}— with R⁴, n and n′ as defined previously, R⁵ is as defined for R⁴ or else R⁴ and R⁵ form, together with the nitrogen atom that bears them, an optionally substituted heterocycloalkyl or heteroaryl such as morpholino, which may be identical or different, being equal to 0 or 1, xiv) hydroxyl, or xv) thiol;
· R″¹, R″² or R″³, which may be identical or different, are as defined for R¹, R² and R³, preferably are chosen from a hydrogen atom or R⁴—Y— with R⁴ being as previously defined and preferably a phenyl group;
· or else R and R¹, which are contiguous, form, together with the carbon atoms which bear them, an optionally unsaturated and optionally substituted (hetero)cycloalkyl group, preferably cycloalkyl which is optionally substituted, in particular with one or more oxo groups and/or optionally fused with an aryl group such as benzo;
· or else two contiguous substituents R¹, R² and/or R′¹, R′² together form a group derived from maleic anhydride such as —C(X)—X—C(X)—;
· X, which may be identical or different, represents an oxygen or sulfur atom or a group NR⁵ with R⁵ as defined previously, preferably representing a hydrogen atom or a (C₁-C₁₀)alkyl group; more particularly, X represents an oxygen atom;
· Y is as defined for X; preferably, Y represents a sulfur atom;
· Metal represents a transition metal such as iron or chromium, preferably Fe, said metal possibly being cationic, in which case the initiator of formula (D) comprises a number of anionic counterions An⁻ as defined previously, making it possible to achieve the electrical neutrality of the molecule;
· L and L′, which may be identical or different, represent a transition metal ligand preferably chosen from the following electron donors: C(X) with X as defined previously, cyano CN, (C₁-C₆)alkenyl, optionally substituted (hetero)aryl, such as bipyridinyl, amines such as the amines R⁴R⁵R⁶N with R⁴ and R⁵ as defined previously and R⁶ representing a hydrogen atom, or a group as defined for R₄, phosphine R⁴R⁵R⁶P such as tri(hetero)arylphosphine, (hetero)cycloalkyl which is preferably unsaturated such as cyclopentadiene, carbene such as arduengo carbenes;
· q represents an integer inclusively between 1 and 6, making it possible to achieve stability of the metal complex, i.e. so as to obtain an electron number around the Metal equal to 16 or 18 electrons (this is also referred to as a 16- or 18-electron coordination sphere);
· R′ and R″, which may be identical or different, represent an optionally substituted (hetero)aryl group;
· An- represents an anionic counterion as defined previously, preferably chosen from (Hal)₆P⁻′ or (Hal)₆Sb⁻, with Hal, which may be identical or different, representing a halogen atom such as fluorine; and
· R^{a}, R^{b}, R^{c} or R^{d}, which may be identical or different, represent a hydrogen atom or a (C₁-C₁₀)alkyl group.

Radical initiators ix) that may also be mentioned include the photoactive dye compounds known as “photosensitizing dyes”, such as ethyl eosin, eosin Y, fluorescein, rose bengal, methylene blue, erythrosine, phloxime, thionine, riboflavin and methylene green.

According to a particular embodiment of the invention, a combination of initiating compounds is used.

According to a particularly advantageous embodiment of the invention, the free-radical initiator(s) ix) which are useful for the polymerization are of formula (A) or (B) as defined previously. More particularly, the free-radical initiator(s) ix) are of formula (A).

According to another particular embodiment of the invention, the free-radical initiator(s) ix) which are useful for the polymerization are of formula (B) as defined previously, preferentially with Xa representing a group O, R with R₁ and R′ with R₃ form, together with the carbon atom that bears them, an oxo group and R₂ and R₄ represent a group R₅—(O)_{w}— in which w is equal to 1, R⁵ representing an optionally substituted (hetero)aryl group such as phenyl or a (hetero)cycloalkyl group which is optionally substituted, notably with a (C₁-C₆)alkyl group such as cyclohexyl optionally substituted with a (C₁-C₆)alkyl group.

According to a particular embodiment of the invention, the radical initiator ix) is AIBN (azobisisobutyronitrile). This initiator generates free radicals i) under the influence of heat at a temperature of greater than or equal to 45° C., preferentially at a temperature of greater than or equal to 55° C., more particularly at 60° C.; and/or ii) photochemically.

According to another particular embodiment of the invention, the radical initiator ix) is ABDV (2,2′-azobis(2,4-dimethylvaleronitrile)). This agent can be used under thermally “milder” polymerization conditions.

Preferentially, when ABDV is used, the polymerization process is performed at a temperature of greater than or equal to 28° C. and preferentially at a temperature of greater than or equal to 35° C., such as 40° C.

The polymerization is preferentially performed in the presence of initiators ix) chosen from peroxide or azo initiators, redox couples and photochemical initiators. Mention may notably be made more preferentially of initiators ix) chosen from:
peroxides, in particular chosen from tert-butyl peroxy-2-ethylhexanoate: Trigonox 21S; 2,5-dimethyl-2,5-bis(2-ethylhexanoylperoxy)hexane: Trigonox 141; tert-butyl peroxypivalate: Trigonox 25C75 from AkzoNobel, or alkali metal or alkaline-earth metal persulfate such as potassium persulfate (KPS); or
azo compounds, in particular chosen from AIBN: azobisisobutyronitrile; V50: 2,2′-azobis(2-amidinopropane) dihalide such as dihydrochloride.

The polymerization is preferably performed at a temperature of between 0° C. and 80° C. and more particularly between 25° C. and 70° C.

According to a particular embodiment, the polymerization is performed at low temperature, i.e. at a temperature of between 1 and 5° C.

### Method for Preparing the Dispersion (A) and the Material (C)

Without this being limiting, in general, the dispersion according to the instant disclosure may be prepared in the following manner:
The polymerization is performed in “dispersion” by precipitation of the polymer being formed, with protection of the formed particles with one or more stabilizers ii) as defined previously, preferably only one type of stabilizer ii) chosen from d) and e) as defined previously.
In a first step,
   the stabilizing polymer (or stabilizing agent ii)) is prepared by mixing the monomer (s) constituting the stabilizing polymer d), or e) as defined above with one or more radical initiators ix), in a medium called synthesis medium, and by polymerizing these monomers, the synthesis medium preferably comprises iii) one or more liquid fatty substances as defined above ,; then one adds to the stabilizing polymer ii) thus formed, the monomers constituting a first family of particles i) in particular the monomers a1) and / or a2), and a3), preferably a1) and a3), more preferably a ″ ′ 1) and a3) as defined above, and the polymerization of these monomers is carried out by the presence of radical initiator (s) ix); then- In a second step, a composition (B) is prepared containing:,
      A) monomers such as:
         A1) a mixture of monomers (M) containing:
            monomers a1) as defined above, preferably (C1-C4) (C1-C4) alkyl (alkyl) acrylate in which the (C1-C4) alkyl group (s) are substituted with a hydroxy, such as the monomers of formula (I′) as defined above, more preferably 2-hydroxyethyl acrylate;
            and / or monomers a2) as defined above and
            monomers a3) as defined above, preferably of formula (5) such as (meth) acrylic acids, preferably acrylic acid;
            said mixture (M) preferably containing only monomers a1) and a3); or
         A2) monomers a3) as defined above preferably of formula (5) and more particularly acrylic acid;
         said composition (B) further containing
      B) one or more radical initiators ix) in particular alkali metal or alkaline earth metal persulphate (s) such as potassium persulphate,
      C) optionally one or more polyols iv) liquid at 20° C. and 1 atmosphere as defined above, preferably chosen from (C2-C6) alkanediol and (C2-C6) alkane -triol such as glycerol and / or water v),
      D) optionally one or more crosslinkers vii) as defined above, in particular chosen from (VII6), (VII′6), (VII₇) and (VII8), such as Ethylene Glycol Diacrylate, Tetra (ethylene glycol) diacrylate, and diacrylate of diethylene glycol (DEGDA), preferably DEGDA,
      E) optionally one or more neutralizers viii) in particular chosen from organic or inorganic alkaline agents, preferably inorganic such as sodium hydroxide or potassium hydroxide, possibly water v),
         it being understood that composition (B) contains C) and / or water v), this composition (B) is added to the medium obtained in the preceding step, the polymerization of these monomers being preferably carried out with stirring and preferably with heating for a period of particularly between 1 minute and 1 day, more particularly between 30 minutes and 10 hours, such as 7 hours at a temperature below 100° C. and above 70° C. such as 90° C.; then the medium is allowed to return to ambient temperature; then
         optionally, when composition (B) does not contain a polyol, it is added during the 1st step or during a 3rd step consisting in adding to the medium one or more polyol (s) liquid (s) at 20° C. and 1 atmosphere iv ) as defined above, preferably chosen from (C2-C6) alkanediol and (C2-C6) alkane-triol such as glycerol. ;
         being It is therefore understood that said method comprises at least one step implementing at least one polyol iv) as defined above, either in the 1st step, or in the 2nd step, or in the 3rd step.

According to one embodiment, composition (B) preferably comprises A + B + D and optionally C and / or E and water v);

According to a particular embodiment, composition (B) comprises: A + B + C + D + E and water v).

According to a particular embodiment, composition (B) comprises A + B + C + D + E and does not contain water v).

In particular, the synthesis medium of the 1st step is nonpolar and volatile organic, preferably chosen from alkanes such as heptane, cyclohexane, Cetiol UT or the mixture of C9-C12 alkanes, preferably of natural origin, in particular linear or branched alkanes, C9-C12. This latter mixture is in particular known under the name INCI C9-C12 ALKANE E511470, CAS 68608-12-8, VEGELIGHT SILK® sold by BioSynthlsVegelight Silk or isododecane, preferably isododecane; or a mixture of:
an apolar solvent as defined above, in particular isododecane;
a polar solvent chosen in particular from esters such as (C1-C4) alkylate (C1-C4) alkyl, for example ethyl acetate;
when the synthesis of the 1st step is completed, the additional solvent is removed and a mixture of i) and ii) is obtained in the liquid fatty substance;

When the synthesis medium from step 1 is a volatile liquid hydrocarbon fatty substance iii), the polymerization can be carried out directly in said oil which therefore also acts as a synthesis solvent.

According to a particular form of the invention, the synthesis medium of the 1st step is a mixture of liquid fatty substances such as iii) and in particular isododecane with an additional solvent chosen in particular from esters with a hydrocarbon-based or branched aliphatic chain, having from 3 to 8 carbon atoms in total such as ethyl acetate, methyl acetate, propyl acetate, n-butyl acetate and in particular ethyl acetate, said additional solvent being chosen to have a boiling point lower than that of the liquid fatty substance. When the synthesis of the stabilizing agents ii) and of the particles i) from step 1 is completed, the additional solvent is removed and a mixture i) and ii) is obtained in the liquid fatty substance.

The monomers are preferably present in the synthesis medium of the 1st step, before polymerization, in an amount of 15% to 45% by weight. All of the monomers can be present in the solvent before the start of the reaction, or part of the monomers can be added as the polymerization reaction progresses.

The polymerization is preferably carried out at a temperature ranging from 70° C. to 110° C. and at atmospheric pressure.

In the event of the presence of cosmetic active agent (s) vi) chosen from f) to j) as defined above in dispersion (A), it (s) little (fri) t be added during the first step; according to another variant, said cosmetic active agent (s) vi) is (are) added during the 2nd step or after the 2nd step; according to another variant, said cosmetic active agent (s) vi) is (are) added during the 3rd step or after the 3rd step, preferably during the 3rd step; if the active ingredient (s) are soluble in water, they are preferably found in composition (B) if said composition (B) as defined above contains water v).

According to one embodiment, composition (B) contains A1) a mixture of monomers (M) containing:
monomers a1) as defined above, preferably (C1-C4) (C1-C4) alkyl (alkyl) acrylate in which the (C1-C4) alkyl group (s) are substituted with a hydroxy, such as the monomers of formula (I′) as defined above, more preferably 2-hydroxyethyl acrylate;
and / or monomers a2) as defined above
And monomers a3) as defined above, preferably of formula (5) such as (meth) acrylic acids, preferably acrylic acid;
said mixture (M) preferably containing only monomers a1) preferably chosen from monomers of formula (I′) more preferably 2-hydroxyethyl (meth) acrylate and monomers a3) preferably chosen from monomers of formula (I′) more preferably acrylic acid.

According to one embodiment, composition (B) contains A2) monomers a3) as defined above preferably of formula (5) and more particularly acrylic acid.

The stabilizing agent ii) is preferably present in the mixture before polymerization of the monomers of the polymer of the particles i). However, it is also possible to add it continuously, in particular when the monomers of the particles i) are also added continuously.

One can use from 0.1% to 50% by weight, particularly from 0.3 to 30% by weight, more particularly from 0.5% to 10% by weight of the stabilizing agent (s) relative to to the total weight of monomers used (stabilizing agents ii) + polymer particles i)), and preferably from 0.9% to 6.3% by weight, relative to the total weight of monomers used.

The dispersion of polymer particles (A) advantageously comprises from 8% to 40% by weight of dry matter, relative to the total weight of said dispersion, and preferably from 15% to 30% by weight per relative to the total weight of said dispersion.

The composition according to the invention preferably comprises a dry matter content (or active material) of polymer particles i) + dispersant polymers ii) ranging from 5% to 60% by weight, relative to the weight total of the dispersion (A), and preferably ranging from 8% to 40% by weight, in particular 15% to 30% by weight, relative to the total weight of the dispersion (A).

In a particular method of preparation, is prepared, in a first step, the statistical stabilizing polymer ii). This stabilizing polymer is soluble in an organic nonpolar alkane solvent such as isododecane.

Then, the at least two particles of different chemical composition are sequentially synthesized i) in the presence of the stabilizing polymer ii).

The stabilizing polymer ii) can be prepared by radical polymerization optionally in the presence of polymerization initiator vi) as defined above.

According to one form of the invention, the particles i1) are first prepared from the polymerization of monomers a1) and / or a2), and a3), preferably a1) and a3), more preferably a″′1) and a3) in the first step then the particles i2) resulting from the polymerization of a mixture of monomers a1) and / or a2), and a3) different from that used for i1 are prepared ), or of monomers a3) as defined above.

The dispersions (A) according to the invention are therefore ultimately formed of polymer particles, of fairly large diameter (preferably greater than 50 nm), and will lead to film-forming deposits, and resistant to fatty substances at the observation temperature (25° C.).

In addition, said dispersion being in an oily medium and may contain water v), it becomes easy to formulate it in cosmetic compositions based on oily medium commonly used in cosmetics, in particular in the phases fatty emulsions, but also in the aqueous phases of emulsions to make it possible to dissolve the water-soluble or hydrophilic active agents.

According to a particular embodiment of the invention, the method is carried out according to the following 3 steps:
For the preparation of these new dispersions, the following process can also be implemented:

### The Polymer Material (C)

The invention also relates to a polymeric material (C), obtained by evaporation of water v) — if present in the medium — and of the liquid fatty substance (s) iii) of the dispersion (A ) as defined above.

According to a particular embodiment of the invention, the material (C) comprises one or more polyols in an amount of greater than or equal to 5% by weight and less than 95% by weight relative to the total weight of the material; in particular, the amount of polyol(s) is between 10% and 90% by weight relative to the total weight of the material, more particularly between 20% and 85% by weight, more particularly between 30% and 80% by weight, relative to the total weight of the material (C).

### Process for Treating Keratin Materials Using the Dispersion (A)

According to an advantageous variant of the invention, the process of the invention is a process for treating keratin fibres, in particular human keratin fibres, preferably the hair, which involves the application to said fibres of at least one dispersion (A) as defined previously.

According to a particular embodiment of the invention, the process for treating keratin materials is a process for treating the skin and/or the eyelashes or the eyebrows involving the application to the skin and/or the eyelashes or the eyebrows of the dispersion (A) as defined previously, followed by a step of drying naturally or of drying using standard devices used in cosmetics as defined previously, preferably naturally.

According to a particular embodiment of the invention, the process for treating keratin materials is a process for making up the skin and/or the eyelashes or the eyebrows using a step of applying the dispersion (A) optionally comprising at least one cosmetic active agent vi) chosen from the ingredients f) to j) as defined previously, notably chosen from the pigments g), and the skin-active agents h), in particular moisturizers such as glycerol and/or antioxidants, preferably moisturizers such as glycerol, and optionally comprising at least one neutralizer viii) as defined previously.

The dispersion (A) according to the invention may also comprise a cosmetic additive chosen from fragrances, preserving agents, fillers, waxes, vitamins, ceramides, free-radical scavengers, polymers other than a), b), c), d) and e), and thickeners.

Preferentially, the first step of the process for treating keratin materials of the invention is the application of the dispersion (A) to the keratin materials, and said dispersion is then left to dry naturally.

According to an advantageous variant, the polymer material (C) is obtained directly on the substrate, in particular on the keratin materials, notably the skin, once the water, the solvent(s) and the liquid fatty substances have evaporated off naturally or dried using a device that is suitable for drying keratin materials, notably the skin, such as a hairdryer, from the dispersion (A).

According to an advantageous variant, once the dispersion (A) has been applied to the keratin materials, notably the skin, it is left to dry for at least 30 seconds, particularly between 1 minute and 30 minutes, more particularly between 5 minutes and 10 minutes.

According to another advantageous variant, the dispersion (A) is applied using a spatula notably without stirring the dispersion beforehand, until filling and levelling of the surface with said spatula is obtained, the excess is removed mechanically using said spatula, followed by a step of drying or of leaving to dry naturally as defined previously.

According to a particular embodiment of the invention, the process involves an additional step of removing the polymer material (C) by scraping, notably with the nail of one hand, the surface of the part treated with the dispersion (A) during the first or second step, followed by removing said polymer material (C) like a film between the thumb and the index finger.

The dispersion (A) according to the invention finds an application in caring for and/or making up the skin and/or the lips and/or for care, notably as an anti-wrinkle treatment.

### Filler(s)

The skin tightening composition may include one filler typically in an amount of about 1 to about 20 wt.%, based on the total weight of the skin tightening composition. The filler may be present in the skin tightening composition in an amount of about about 1 to about 20 wt. %, about 1.5 to about 20 wt. %, about 2 to about 20 wt. %, about 2.5 to about 20 wt.%, about 3 to about 20 wt.%, about 3.5 to about 20 wt.%, about 4 to about 20 wt. %, about 4.5 to about 20 wt. %, about 5 to about 20 wt. %, about 6 to about 20 wt. %, about 7 to about 20 wt. %, about 8 to about 20 wt.%; about 1.5 to about 13 wt. %, about 1 to about 13 wt. %,about 1.5 to about 13 wt. %, about 2 to about 13 wt.%, about 2.5 to about 13 wt. %, about 3 to about 13 wt. %, about 3.5 to about 13 wt.%, about 4 to about 13 wt. %, about 4.5 to about 13 wt. %, about 5 to about 13 wt. %, about 6 to about 13 wt. %, about 7 to about 13 wt. %, about 8 to about 13 wt.%; about 0.5 to about 11 wt. %, about 1 to about 11 wt. %, about 1.5 to about 11 wt. %, about 2 to about 11 wt. %, about 2.5 to about 11 wt. %, about 3 to about 11 wt. %, about 3.5 to about 11 wt. %, about 4 to about 11 wt. %, about 4.5 to about 11 wt. %, about 5 to about 11 wt.%, about 6 to about 11 wt. %, about 7 to about 11 wt. %, about 8 to about 11 wt.%; about 0.5 to about 9 wt. %, about 1 to about 9 wt. %, about 1.5 to about 9 wt. %, about 2 to about 9 wt. %, about 2.5 to about 9 wt. %, about 3 to about 9 wt. %, about 3.5 to about 9 wt. %, about 4 to about 9 wt. %, about 4.5 to about 9 wt. %, about 5 to about 9 wt. %, about 6 to about 9 wt. %, about 7 to about 9 wt.%; about 0.5 to about 7 wt. %, about 1 to about 7 wt. %, about 1.5 to about 7 wt. %, about 2 to about 7 wt. %, about 2.5 to about 7 wt. %, about 3 to about 7 wt. %, about 3.5 to about 7 wt. %, about 4 to about 7 wt. %, about 4.5 to about 7 wt.%, or about 5 to about 7 wt.%, including all ranges and subranges thereof, based on the total weight of the skin tightening composition.

The one or more filler may be hydrophobic silica (such as, silica sylilate), silica, nylon-12, cellulose, methacrylate crosspolymer (such as, methyl methacrylate crosspolymer), silicone powder (such as, polymethylsisesquioxane), and a combination thereof.

In some instances, the skin tightening composition preferably includes a hydrophobic silica, such as silica silylate. Hydrophobic silica are often provided in the form of particles, porous material obtained by replacing (by drying) the liquid component of a silica gel with air. They are generally synthesized via a sol-gel process in a liquid medium and then dried, usually by extraction with a supercritical fluid; the one most commonly used being supercritical CO₂. This type of drying makes it possible to avoid shrinkage of the pores and of the material. In some cases, the particles dissolve when combined with solvents.

The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalize the OH groups with silyl groups Si--Rn, for example trimethylsilyl groups. As regards the preparation of hydrophobic silica aerogel particles modified at the surface by silylation, reference may be made U.S. Pat. No. 7,470,725, incorporated herein by reference in its entirety. In some cases, useful hydrophobic silica particles are surface-modified with trimethylsilyl groups. In at least one instance, the hydrophobic silica is aerogel.

Hydrophobic silica include those that exhibit a specific surface area per unit of mass (SM) ranging from 500 to 1500 m²/g, from 600 to 1200 m²/g, or 600 to 800 m²/g, and a size, expressed as the volume-mean diameter (D[0.5]), ranging from 1 to 1500 µm, from 1 to 1000 µm, or from 1 to 100 µm, in particular from 1 to 30 µm, from 5 to 25 µm, or from 5 to 20 µm, and in some cases from 5 to 15 µm. In some cases, the hydrophobic silica particles used in the skin tightening composition have a size, expressed as the volume-mean diameter (D[0.5]), ranging from 1 to 30 µm, from 5 to 25 µm, from 5 to 20 µm or from 5 to 15 µm. In some instances, the hydrophobic silica particles have a specific surface area per unit of mass (SM) ranging from 600 to 800 m²/g and a size expressed as the volume-mean diameter (D[0.5]) ranging from 5 to 20 µm or from 5 to 15 µm.

The specific surface area per unit of mass can be determined by the nitrogen absorption method, known as the BET (Brunauer-Emmett-Teller) method, described in The Journal of the American Chemical Society, Vol. 60, page 309, February 1938 and corresponding to the international standard ISO 5794/1 (appendix D). The BET specific surface area corresponds to the total specific surface area of the particles under consideration.

The sizes of hydrophobic silica particles (e.g., aerogel particles) can be measured by static light scattering using a commercial particle size analyzer of MasterSizer 2000 type from Malvern. The data are processed on the basis of the Mie scattering theory. This theory, which is exact for isotropic particles, makes it possible to determine, in the case of non-spherical particles, an “effective” particle diameter. This theory is described in particular in the publication by Van de Hulst, H. C., “Light Scattering by Small Particles”, Chapters 9 and 10, Wiley, New York, 1957.

The hydrophobic silica particles may advantageously have a tapped density ρ ranging from 0.02 g/cm³ to 0.10 g/cm³, from 0.03 g/cm³ to 0.10 g/cm³, from 0.04 g/cm³ to 0.10 g/cm³, or from 0.05 g/cm³ to 0.08 g/cm³. The density ρ, known as the tapped density, may be assessed according to the following protocol: 40 g of powder are poured into a measuring cylinder; the measuring cylinder is then placed on a Stay 2003 machine from Stampf Volumeter; the measuring cylinder is then subjected to a series of 2500 packing motions (this operation is repeated until the difference in volume between two consecutive tests is less than 2 percent); the final volume Vf of packed powder is then measured directly on the measuring cylinder. The tapped density is determined by the ratio w/Vf, in this instance 40/Vf (Vf being expressed in cm³ and w in g).

In some cases, the hydrophobic silica particles (e.g., aerogel particles) may have a specific surface area per unit of volume SV ranging from 5 to 100 m²/cm³, from 10 to 90 m²/cm³, from 15 to 40 m²/cm³, from 20 to 85 m²/cm³, or from 24 to 80 m²/cm³. The specific surface area per unit of volume is given by the relationship: S_{V}=S_{M}×ρ, where ρ is the tapped density, expressed in g/cm³, and S_{M} is the specific surface area per unit of weight, expressed in m²/g, as defined above.

The hydrophobic silica particles may have an oil-absorbing capacity, measured at the wet point, ranging from 5 to 18 ml/g, from 6 to 15 ml/g, or from 8 to 12 ml/g. The absorption capacity measured at the wet point, denoted Wp, corresponds to the amount of oil which is necessary to add to 100 g of particles in order to obtain a homogeneous paste. It is measured according to the “wet point” method or method of determination of oil uptake of a powder described in the standard NF T 30-022.

Mention may be made of hydrophobic silica sold under the name VM-2260 (INCI name: Silica silylate) by Dow Corning, the particles of which have a mean size of approximately 1000 microns and a specific surface area per unit of mass ranging from 600 to 800 m²/g. Mention may also be made of the aerogels sold by Cabot under the references Aerogel TLD 201, Aerogel OGD 201, Aerogel TLD 203, Enova.RTM. Aerogel MT 1100 and Enova Aerogel MT 1200. Additionally, the hydrophobic silica aerogel particles sold under the name VM-2270 (INCI name: Silica silylate), by the company Dow Corning, the particles having an average size ranging from 5-15 microns and a specific surface area per unit of mass ranging from 600 to 800 m²/g may be useful.

The one or more filler may be nylon-12, cellulose, methacrylate crosspolymer (such as, methyl methacrylate crosspolymer), silicone powder (such as, polymethylsisesquioxane), silica, silica silylate, fumed silica and a combination thereof.

Additional fillers may include polyvalent silicates such as magnesium silicate, calcium silicate, aluminum silicate, a polyvalent silicate clay, montmorillonite, bentonite, smectite, and combinations thereof. For instance, the filler may comprise magnesium aluminum silicate.

In one embodiments, the one or more fillers may be chosen from silica silylate, fumed silica, zeolite, natural clay, synthetic clay, kaolin, hectorite, organically modified hectorite, pentaerythrityl tetraisostearate (and) disteardimonium hectorite (and) propylene carbonate, an activated clay, disteardimonium hectorite, stearalkonium hectorite, quaternium-18 bentonite, quaternium-18 hectorite, benzalkonium bentonite, and a mixture thereof.

In one or more embodiments, the weight ratio of the total amount of i) of the dispersion A to the total amount of filler (film forming polymers: filler) is between around 1:1 to 7:1. In one or more embodiments of the skin tightening composition, the weight ratio of the total amount of i) of the dispersion A to the total amount of filler (film forming polymers : filler) is between around 1:1 to 7:1, 1:1 to 6.5:1, 1:1 to 6:1, 1:1 to 5.5:1, 1:1 to 5:1, 1:1 to 4.5:1, 1:1 to 4:1, 1:1 to 3.5:1, 1:1 to 3.5:1, 1:1 to 3:1, 1:1 to 2.5:1, 1:1 to 2:1, 1:1 to 1.5:1, or any ranges and subranges thereof.

### Fatty Compounds

In addition to the volatile hydrocarbon oils mentioned above, the skin tightening compositions may optionally include one or more fatty compounds, in particular, non-volatile fatty compound. The total amount of fatty compounds in the skin tightening compositions, if present, may vary but is typically about 0.1 to about 20 wt.%, based on the total weight of the skin tightening composition. In some cases, the total amount of emulsifying fatty compounds in the skin tightening compositions is about 0.1 to about 15 wt.%, about 0.1 to about 10 wt.%, about 0.1 to about 5 wt.%, about 1 to about 20 wt.%, about 1 to about 15 wt.%, about 1 to about 10 wt.%, about 1 to about 5 wt.%; about 2 to about 20 wt.%, about 2 to about 15 wt.%, about 2 to about 10 wt.%, about 2 to about 5 wt.%; about 4 to about 20 wt.%, about 4 to about 15 wt.%, about 4 to about 10 wt.%, about 4 to about 5 wt.%, including ranges and subranges thereof, based on the total weight of the skin tightening composition.

Non-limiting examples of non-volatile oils include:
i. hydrocarbon oils of animal origin such as perhydrosqualene;
ii. plant hydrocarbon oils, such as liquid triglycerides of fatty acids, for example sunflower oil, corn oil, soybean oil, marrow oil, grapeseed oil, sesame oil, hazelnut oil, apricot oil, macadamia oil, castor oil, avocado oil, caprylic/capric acid triglycerides;
iii. oils of formula R₉COOR₁₀ in which R₉ represents a higher fatty acid residue containing from 7 to 19 carbon atoms and R₁₀ represents a branched hydrocarbon chain containing from 3 to 20 carbon atoms, such as, for example, Purcellin oil;
iv. linear or branched hydrocarbons of mineral or synthetic origin, such as non-volatile liquid paraffins and derivatives thereof, petroleum jelly (petrolatum), polydecenes, and hydrogenated polyisobutene such as parleam;
v. synthetic esters and ethers such as isopropyl myristate, octanoates, decanoates or ricinoleates of alcohols or of polyalcohols;
vi. fatty alcohols such as octyidodecanol or oleyl alcohol;
vii. partially hydrocarbonated and/or siliconated fluoro oils;
viii. silicone oils such as linear, non-volatile polydimethylsiloxanes (dimethicone) which are liquid or pasty at room temperature, phenyldimethicones, phenyltrimethicones and polymethylphenylsiloxanes; and combinations thereof.

In some instances, the one or more fatty substances may be selected form polyolefins (petrolatum), waxes, squalane, squalene, hydrogenated polyisobutene, hydrogenated polydecene, polybutene, mineral oil, pentahydrosqualene, dimethicone, and a combination thereof.

Additional fatty compounds that are worth mentioning include fatty alcohols, fatty esters, fatty alcohols derivatives, fatty acid derivatives, such as those discussed below.

### Fatty Alcohols

The one or more fatty compounds may be glycerolated and/or oxyalkylenated, include from 8 to 30 carbon atoms, and/or be saturated or unsaturated. The fatty alcohols useful herein include those having from about 8 to about 30 carbon atoms, from about 12 to about 22 carbon atoms, and from about 14 to about 22 carbon atoms. These fatty alcohols can be straight or branched chain alcohols and can be saturated or unsaturated. Non-limiting examples of fatty alcohols include decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, cis4-t-butylcyclohexanol, myricyl alcohol and a combination thereof. In some cases, the fatty alcohols comprise at least one of or may be chosen from myristyl alcohol, cetyl alcohol, stearyl alcohol, isostearyl alcohol, oleyl alcohol, isotridecyl alcohol, and a combination thereof.

The saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

The unsaturated liquid fatty alcohol may include in their structure at least one double or triple bond. For example, the fatty alcohols may include several double bonds (such as 2 or 3 double bond), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

Non-limiting examples of solid fatty alcohols include linear or branched, saturated or unsaturated alcohols containing from 8 to 30 carbon atoms, for example, myristyl alcohol, cetyl alcohol, stearyl alcohol and their combination, cetylstearyl alcohol.

### Fatty Esters

The fatty compounds of the skin tightening composition may be liquid or solid fatty esters at 25° C., 1 atm. The fatty esters may include esters from a C₆-C₃₂ fatty acid and/or a C₆-C₃₂ fatty alcohol. For example, the fatty compounds may include or be chosen from fatty acid monoesters and diesters, polyol esters, polyglycerol esters, polyglycerol polyricinoleate, polyglycerol poly-12-hydroxystearate, polyglycerol dimerate isostearate, ethylhexanoate, polyglycerol esters, and a combination thereof. These esters may be esters of saturated or unsaturated, linear or branched C₁-C₂₆ aliphatic mono or polyacids and of saturated or unsaturated, linear or branched C₁-C₂₅ aliphatic mono or polyalcohols, the total number of carbon atoms in the esters being greater than or equal to 10. In one instance, the fatty compounds comprise one or more fatty acid monoesters. For the esters of monoalcohols, at least one of the alcohol or the acid from which the esters result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate and isostearyl neopentanoate.

In some instances, the fatty esters are cetyl esters, such as esters of saturated fatty acids and fatty alcohols. For example, the fatty esters may include or be chosen from cetyl palmitate, cetyl stearate, myristyl myristate, myristyl stearate, cetyl myristate, stearyl stearate, cetearyl ethylhexanoate, and combinations thereof. In one instance, the fatty esters may be one or more of or chosen from isopropyl isostearate, n-propyl myristate, isopropyl myristate, hexyl laurate, hexadecyl isostearate, hexydecyl laurate, hexyldecyl octanoate, n-propyl palmitate, isopropyl palmitate, and combinations thereof. In another instance, the fatty esters include or may be chosen from diisobutyl adipate, 2-hexyldecyl adipate, di-2-heptylundecyl adipate, monoisostearic acid N-alkyl glycol, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprate, triethyl citrate, 2-ethylhexyl succinate, isocetyl stearate, butyl stearate, diisopropyl sebacate, di-2-ethylhexyl sebacate, cetyl lactate, myristyl lactate, isopropyl palm itate, 2-ethylhexyl palm itate, 2-hexyldecyl palm itate, 2-heptylundecyl palm itate, cholesteryl 12-hydroxystearate, dipentaerythritol fatty acid ester, isopropyl myristate, octyldodecyl myristate, 2-hexyldecyl myristate, myristyl myristate, hexyldecyl dimethyloctanoate, ethyl laurate, hexyl laurate, diisostearyl malate, dicaprylyl carbonate, cetearyl ethylhexanoate, and combinations thereof. In yet a further instance, the skin-tightening composition includes one or more of or may have fatty compounds chosen from cetearyl alcohol, cetearyl ethylhexanoate, isopropyl myristate, and combinations thereof.

Non-limiting examples of solid fatty acid esters and/or fatty acid esters that may be mentioned include solid esters obtained from C₉-C₂₆ fatty acids and from C₉-C₂₅ fatty alcohols. Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

Non-limiting examples of liquid fatty acid include triglyceride oils of plant or synthetic origin, such as liquid fatty acid triglycerides containing 6 to 30 carbon atoms, for instance heptanoic or octanoic acid triglycerides, or alternatively, e.g., sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, coconut oil, camellina oil, tamanu oil, babassu oil and pracaxi oil, jojoba oil, shea butter oil, and combinations thereof. In one instance, the one or more fatty compounds include at least one of or are selected from fatty acid triglycerides, oils, mineral oil, alkanes, fatty alcohols, fatty acids, fatty alcohol derivatives, alkoxylated fatty acids, polyethylene glycol esters of fatty acids, propylene glycol esters of fatty acids, butylene glycol esters of fatty acids, esters of neopentyl glycol and fatty acids, polyglycerol/glycerol esters of fatty acids, glycol diesters, diesters of ethylene glycol and fatty acids, esters of fatty acids and fatty alcohols, esters of short chain alcohols and fatty acids, esters of fatty alcohols, hydroxy-substituted fatty acids, waxes, and a combination thereof. In another instance, fatty compounds of the skin-tightening composition includes one or more fatty acid triglycerides, such as caprylic/capric triglyceride.

### Fatty Alcohol Derivatives

The skin-tightening compositions may, in some instances, include fatty alcohol derivatives such as alkyl ethers of fatty alcohols, alkoxylated fatty alcohols, alkyl ethers of alkoxylated fatty alcohols, esters of fatty alcohols and a combination thereof. Non-limiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcochol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a combination of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a combination thereof. Liquid fatty ethers may be chosen from liquid dialkyl ethers such as dicaprylyl ether. The non-liquid fatty ethers may also be chosen from dialkyl ethers and in particular dicetyl ether and distearyl ether, alone or as a combination.

Esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate; diisopropyl sebacate; diisopropyl adipate; di-n-propyl adipate; triisopropyl citrate; glyceryl trilactate; glyceryl trioctanoate; neopentyl glycol diheptanoate; and diethylene glycol diisononanoate.

### Fatty Acid Derivatives

The skin-tightening compositions may, in some instances, include fatty acid derivatives. The fatty acid derivatives are defined herein to include fatty acid esters of the fatty alcohols as defined above, fatty acid esters of the fatty alcohol derivatives as discussed above when such fatty alcohol derivatives have an esterifiable hydroxyl group, fatty acid esters of alcohols other than the fatty alcohols and the fatty alcohol derivatives described above, hydroxy-substituted fatty acids, and a combination thereof. Non-limiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a combination thereof.

### Non-Ionic Surfactant(s)

The skin tightening composition may, optionally, include one or more nonionic surfactants. Although the skin tightening composition is typically an emulsion when containing one or more nonionic surfactants, the skin tightening composition may alternatively be anhydrous when containing such nonionic surfactants.

The nonionic surfactant(s) may include one or more of peg-30 dipolyhydroxystearate, polyglyceryl-4 diisostearate/polyhydroxystearate/sebacate, polyglyceryl-4 isostearate, polyglyceryl-2 dipolyhydroxystearate, dimethicone (and) peg/ppg-18/18 dimethicone, lauryl peg-9 polydimethylsiloxyethyl dimethicone, and a combination thereof. The nonionic surfactant can be, for example, selected from alcohols, alpha-diols, alkylphenols and esters of fatty acids, these compounds being ethoxylated, propoxylated or glycerolated and having at least one fatty chain comprising, for example, from 8 to 18 carbon atoms. The number of ethylene oxide or propylene oxide groups of the foregoing compounds may range from 2 to 50, and the number of glycerol groups may range from 1 to 30. Mention may be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C6-C24)alkylpolyglycosides; N-(C6-C24)alkylglucamine derivatives, amine oxides such as (C10-C14)alkylamine oxides or N-(C10-C14)acylaminopropylmorpholine oxides; and combinations thereof. Maltose derivatives may also be mentioned.

The nonionic surfactants may be chosen from polyoxyalkylenated or polyglycerolated nonionic surfactants. The oxyalkylene units are more preferably oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and combinations thereof.

Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups—such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and combinations thereof. As glyceryl esters of fatty acids, glyceryl stearate (glyceryl mono-, di- and/or tristearate) (CTFA name: glyceryl stearate) or glyceryl ricinoleate and combinations thereof can in particular be cited. As glyceryl esters of C₈-C₂₄ alkoxylated fatty acids, polyethoxylated glyceryl stearate (glyceryl mono-, di- and/or tristearate) such as PEG-20 glyceryl stearate can for example be cited. Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

Typically, the amount of nonionic surfactants included in the skin tightening compositions, when present, ranges from about 0.5 to about 10 wt.%, about 0.5 to about 8 wt.%, about 0.5 to about 6 wt.%, about 0.5 to about 5 wt.%, about 0.5 to about 4 wt.%;about 1 to about 10 wt.%, about 1 to about 8 wt.%, about 1 to about 6 wt.%, about 1 to about 5 wt.%, about 1 to about 4 wt.%; about 2 to about 10 wt.%, about 2 to about 8 wt.%, about 2 to about 6 wt.%, about 2 to about 5 wt.%, about 2 to about 4 wt.%; about 3 to about 10 wt.%, about 3 to about 8 wt.%, about 3 to about 6 wt.%, about 3 to about 5 wt.%, about 3 to about 4 wt.%, including all ranges and subranges thereof, based on the total weight of the skin tightening composition.

### Dispersant(s)

The skin tightening composition may, optionally, include one or more dispersant. The dispersant may be chosen from olyoxyethylene glycol ethers or esters (POE/PEG ethers or esters) or polyoxypropylene glycol ethers or esters (PPG ethers or esters), from sugar ethers or esters, from glycerol or polyglycerol ethers or esters and from ethoxylated glyceride esters (POE glyceryl esters), polyhydroxystearic acid, or a combination thereof.

The dispersant may be selected such that it protects various ingredients of the skin tightening composition, such as coloring particles, that are solid at room temperature and atmospheric pressure against their aggregation or flocculation when it is placed in contact with an aqueous composition. More generally, the dispersant may be a surfactant, an oligomer, a polymer or a combination of several thereof, bearing one or more functionalities that have strong affinity for the surface of the compounds to be dispersed. In some instances, the dispersant may be physically adsorbed onto the surface of the particles to be dispersed. In at least one instance, the dispersant is selected from those having a hydrophilicity with an HLB of 10 or less, 7 or less, or 6 or less. The term “HLB of 10 or less” means a surfactant having, at 25° C., an HLB balance (hydrophilic-lipophilic balance), within the Griffin meaning, of less than or equal to 10. The dispersant may be nonionic and/or chosen from polyoxyethylene glycol ethers or esters (POE/PEG ethers or esters) or polyoxypropylene glycol ethers or esters (PPG ethers or esters), from sugar ethers or esters, from glycerol or polyglycerol ethers or esters and from ethoxylated glyceride esters (POE glyceryl esters) or from combinations thereof.

Typically, the amount of dispersants included in the skin tightening compositions, when present, ranges from about 0.1 to about 10 wt.%, 0.5 to about 10 wt.%, about 0.5 to about 8 wt.%, about 0.5 to about 6 wt.%, about 0.5 to about 5 wt.%, about 0.5 to about 4 wt.%;about 1 to about 10 wt.%, about 1 to about 8 wt.%, about 1 to about 6 wt.%, about 1 to about 5 wt.%, about 1 to about 4 wt.%; about 1.5 to about 10 wt.%, about 1.5 to about 8 wt.%, about 1.5 to about 6 wt.%, about 1.5 to about 5 wt.%, about 1.5 to about 4 wt.%, including all ranges and subranges thereof, based on the total weight of the skin tightening composition.

### Water-Soluble Solvents

The skin tightening composition may optionally include one or more water-soluble solvents. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water soluble solvent has a solubility of at least 60%, 70%, 80%, or 90%. Non-limiting examples of water-soluble solvents include, for example, glycerin, C₁₋₄ alcohols, organic solvents, fatty alcohols, fatty ethers, fatty esters, polyols, glycols, and any a combination thereof. In some instances, the skin tightening composition includes one or more C₁₋₄ alcohols, for example, ethanol.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a combination thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a combination thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a combination thereof.

The total amount of the water-soluble solvents in the skin tightening composition, if present, may vary but is typically about 0.01 to about 25 wt.%, based on the total weight of the skin tightening composition. In some cases, the total amount of water-soluble solvents is about 0.01 to about 20 wt.%, about 0.01 to about 15 wt.%, about 0.01 to about 10 wt.%, about 0.1 to about 25 wt.%, about 0.1 to about 20 wt.%, about 0.1 to about 15 wt.%, about 0.1 to about 10 wt.%, about 1 to about 25 wt.%, about 1 to about 20 wt.%, about 1 to about 15 wt.%, about 1 to about 10 wt.%, or about 1 to about 8 wt.%, including all ranges and subranges thereof, based on the total weight of the skin tightening composition.

### Soft Focus Powder

The skin tightening compositions may, optionally, include soft focus powder. Soft focus powders are materials providing a blurring effect, typically due to their light-scattering properties on the skin. Such powders typically have high diffuse reflectance, low specular reflectance, and high diffuse transmittance. Soft focus powders give the skin a smoother appearance, for example, by reducing the difference in luminosity between the valley and the edges of wrinkles and imperfections.

Non-limiting examples of soft focus powders include powders of natural or synthetic origin such as mica, titanated mica, alumina, titanium dioxide, serecite, composite talc/titanium dioxide/alumina/silica powders, polyamide, poly(methyl (meth)acrylate), polyethylene powder, polymethylsilsesquioxane powder, sodium acrylates crosspolymer-2 and a combination thereof. Additional non-limiting examples include calcium aluminum borosilicate (LUXSIL), PMMA (Microsphere M-100), polyethylene (POLYETHYLENE Cl 2080), methyl methacrylate crosspolymer (COVABEADS LH85), nylon-12 (ORGASOL 2002), or ethylene/acrylic acid copolymer (FLOBEADS EA209). In some instances, the skin tightening compositions include at least one soft focus powder selected from the group consisting of silica which may or may not be coated, fumed silica, silica silylate, composite talc/titanium dioxide/alumina/silica powders, polyamide (nylon), poly(methyl (meth)acrylate), polyethylene powder, polymethylsilsesquioxane powder, waxes, such as copernicia cerifera (carnauba) wax, dimethicone/vinyl dimethicone crosspolymer, nylon-12, cellulose, polylactic acid, boron nitride, and a combination thereof. The copernicia cerifera (carnauba) wax can be provided as a dispersion non-water and alcohol. The dimethicone/vinyl dimethicone crosspolymer can be provided as silicone dispersion (INCI: Dimethicone/vinyl dimethicone crosspolymer (and) C12-14 Pareth-12). In some instances, the soft focus powder is (or includes) sodium acrylates crosspolymer-2, which is commercially available as AQUAKEEP 10SH-NFC as sodium acrylates crosspolymer-2 (and) water (and) silica.

The total amount of soft focus powder, if present, can vary but is typically about 0.1 to about 20 wt.%, based on the total weight of the skin tightening composition. In some cases, the total amount of soft focus powder is about 0.1 to about 20 wt.%, about 0.1 to about 18 wt.%, about 0.1 to about 16 wt.%, about 0.1 to about 15 wt.%, about 0.1 to about 14 wt.%, about 0.1 to about 12 wt.%, about 0.1 to about 10 wt.%, about 0.1 to about 5 wt.%, about 1 to about 20 wt.%, about 1 to about 18 wt.%, about 1 to about 16 wt.%, about 1 to about 15 wt.%, about 1 to about 14 wt.%, about 1 to about 12 wt.%, about 1 to about 10 wt.%, or about 1 to about 8 wt.%, about 2 to about 20 wt.%, about 2 to about 18 wt.%, about 2 to about 16 wt.%, about 2 to about 14 wt.%, about 2 to about 12 wt.%, about 2 to about 10 wt.%, about 2 to about 8 wt.%, about 4 to about 20 wt.%, about 4 to about 18 wt.%, about 4 to about 16 wt.%, about 4 to about 14 wt.%, about 4 to about 12 wt.%, about 4 to about 10 wt.%, about 4 to about 8 wt.%, including ranges and subranges thereof, based on the total weight of skin tightening composition.

### Optional Components

In one or more embodiments, the skin tightening compositions described herein may contain one or more additional ingredients (additives and miscellaneous ingredients). Examples include, but are not limited to surfactants, emulsifiers, thickeners (such as polysaccharide-based thickeners), other polymers, proteins, hydrolyzed proteins, amino acids, fragrance, pH adjusters, and preservatives. Additional details regarding such additional ingredients follows below.

The skin tightening composition according to the disclosure can comprise any additional ingredients suitable for use in skin tightening compositions. Such ingredients may include, but are not limited to, cosmetically acceptable solvents, silicone compounds, rheology modifying agents such as acrylic polymers, cationic, nonionic, amphoteric or zwitterionic surfactants or combinations thereof, anionic, cationic, nonionic, amphoteric or zwitterionic polymers or combinations, humectants and moisturizing agents, fatty substances other than the claimed fatty substances, emulsifying agents other than fatty substances, fillers, structuring agents, propellants, shine agents, conditioning agents, antioxidants or reducing agents, penetrants, sequestrants, fragrances, buffers, dispersants, conditioning agents, for instance volatile or non-volatile, modified or unmodified silicones, ceramides, preserving agents, opacifiers, sunscreen agents, and antistatic agents. Acids, for example citric acid, can affect the pH of the system resulting in loss of lift.

The skin tightening composition may also contain acid and alkali pH adjusters, which are well known in the art. Such pH adjusters include, but are not limited to, sodium metasilicate, silicate compounds, citric acid, ascorbic acid, and carbonate compounds.

### Methods

The skin tightening compositions are particularly useful for improving the appearance of skin, especially the skin of a human. When the skin tightening compositions are applied to the skin, they provide an immediate improvement to the appearance of the skin that is long lasting. The skin tightening compositions are particularly useful for method of:
reducing the appearance of fine lines of the skin;
reducing the appearance of wrinkles of the skin;
improving the tone of skin and/or improving the evenness of skin tone;
improving skin softness and/or smoothness;
reducing the appearance of eye bags;
reducing the appearance of dark circles around and/or below the eyes;
reducing the appearance of pores and/or scars; and/or
increasing the radiance, luminosity, and/or glow of the skin.

Typically, an effective amount of a skin tightening composition is applied to the skin to be treated, for example, the skin of the face and/or neck. In some instances, it may be desirable to apply the skin tightening composition to the skin around (or below) the eyes. The skin tightening compositions can be applied with the hands or may be applied using a brush, sponge, tissue, cotton swab, fabric, or applicator (e.g., pen or other device), etc. The amount needed to achieve the desired effect can be ascertained by the consumer.

### EXAMPLES

Implementation of the present disclosure is provided by way of the following examples. The following examples serve to elucidate aspects of the technology without being limiting in nature.

In each of the following examples, the amounts of components given are in terms of active material (AM).

### Example 1

### Step 1: Preparation of the Oily Dispersion (A) Comprising i) and Stabilizer ii), Acrylic Acid, Methyl Acrylate, Ethyl Acrylate and Isobornyl Acrylate in Isododecane

The oily dispersions used in Examples 1′ and 2′ below were prepared according to the same preparation process as in Example 4 of International Patent Application WO 2015/091513, the amounts being adapted as defined in Table 1 below.

The compositions of Examples 1′ and 2′ were prepared in the same manner according to Table 1 below. All the percentages of reagents described in the examples are weight percentages, given as g per 100 g of composition.

TABLE 1

| Preparation steps | Ingredients | Example 1′ | Example 2′ |
|---|---|---|---|
| Step 1: Preparation of the oily dispersion | Isobornyl acrylate | 2.1 | 2.2 |
| | Methyl acrylate | 1.1 | 1.1 |
| | Ethyl acrylate | 6.7 | 6.8 |
| | Acrylic acid | 1.1 | 1.1 |
| | Isododecane | 11.0 | 11.2 |
| Sum of the monomers | About 20% | 22 | 22.4 |
| Step 2 Preparation of the aqueous phase preparation B | 2-Hydroxyethyl acrylate | 3.3 | 6.1 |
| | Acrylic acid | 3.3 | 0.7 |
| | Sodium hydroxide | 1.4 | 0.3 |
| | Potassium persulfate | 0.2 | 0.2 |
| | Di(ethylene glycol) diacrylate | 0.7 | 0.6 |
| | Water | 24.0 | 25.9 |

In the second step, sodium hydroxide in pellet form is dissolved in water with magnetic stirring (250 rpm). The exothermic reaction is controlled by means of an ice bath.

The monomer(s) are then gradually added with stirring, while cooling.

Finally, the polymerization initiator and the crosslinking agent, if any, are introduced. The mixture is homogenized with a Speed Mixer 3500 RPM for 2 minutes, twice.

In total, two aqueous compositions B1′ and B2′were prepared. The compositions according to the invention Ex. 1′ were combined with B1′, Ex. 2′ with B2′.

TABLE 2

| Reagents: | Aqueous phase B1′ | Aqueous phase B2′ |
|---|---|---|
| Acrylic acid | 10.05 | 2.00 |
| 2-Hydroxyethyl acrylate | 10.05 | 18.00 |
| Sodium hydroxide | 4.26 | 0.83 |
| Diethylene glycol diacrylate (DEGDA) | 2.18 | 1.76 |
| Potassium persulfate (KPS) | 0.61 | 0.49 |
| Water | qs 100 | qs 100 |
| Acrylic acid | 10.05 | 2.00 |
| 2-Hydroxyethyl acrylate, weight% | 10.05 | 18.00 |
| Molar % neutralization/acrylic acid | 75 | 75 |
| Molar % crosslinking/monomer(s) | 4.5 | 4.5 |
| Molar % initiator/monomer(s) | 1.0 | 1.0 |

Data in g per 100 g of composition unless otherwise indicated (weight%)

### Between Step 2 and Step 3: Addition of the Aqueous Phase to the Oily Dispersion From Step 1

The aqueous phases at 60% by weight are added to 40% by weight of oily dispersion with stirring at room temperature (25° C.). Next, the stirred mixture is heated for a time of several hours (about 7 hours) at a temperature below 100° C. and above 70° C., such as 90° C. The medium is allowed to return to room temperature.

### Example 2 (Inventive Examples)

Exemplary compositions were prepared in accordance with aspects of the disclosure and are reported in Table 3 below. Several prototypes of compositions were prepared at lab scale using the following protocol.

Formulations A-P were prepared using the weight percentages of the materials listed in Table 1 using the following process.
1. Glycerin was incorporated into Dispersion A by mixing the two materials at 2750 rpm for 5 mins in a speedmixer.
2. Polyhydroxystearic acid and isododecane were added to the dispersion from step 1 and mized at 2750 rpm for 3 min.
3. Finally, silica silylate was added and the mixture was further mixed at 2750 rpm for 3 min.
4. The formula was allowed to rest for 18-24 hours before performing any tests. The weight percentage of the Dispersion A and dispersant, polyhydroxystearic acid were kept constant at 53.45% and 1.99%, while the amount of glycerin, isododecane and silica silylate was changed resulting in formulas with different textures and properties.

TABLE 3

Composition of formulas A-P by weight percent of each raw material

| FLA # | GLYCERIN | DISPERSION A | POLYHYDROXYS TEARIC ACID | ISODODECANE | SILICA SILYLATE |
|---|---|---|---|---|---|
| A | 6.50 | 53.45 | 1.99 | 35.03 | 3.03 |
| B | 6.50 | 53.45 | 1.99 | 27.37 | 10.69 |
| C | 6.50 | 53.45 | 1.99 | 35.92 | 2.14 |
| D | 6.50 | 53.45 | 1.99 | 36.72 | 1.34 |
| E | 6.50 | 53.45 | 1.99 | 36.99 | 1.07 |
| F | 6.50 | 53.45 | 1.99 | 33.78 | 4.28 |
| G | 6.50 | 53.45 | 1.99 | 34.77 | 3.29 |
| H | 6.50 | 53.45 | 1.99 | 34.50 | 3.56 |
| I | 9.60 | 53.45 | 1.99 | 31.67 | 3.29 |
| J | 19.25 | 53.45 | 1.99 | 22.02 | 3.29 |
| K | 6.50 | 53.45 | 1.99 | 35.39 | 2.67 |
| L | 9.60 | 53.45 | 1.99 | 32.29 | 2.67 |
| M | 19.25 | 53.45 | 1.99 | 22.64 | 2.67 |
| N | 19.25 | 53.45 | 1.99 | 23.17 | 2.14 |
| O | 19.25 | 53.45 | 1.99 | 23.53 | 1.78 |
| P | 19.25 | 53.45 | 1.99 | 23.97 | 1.34 |

### Example 3 Film Properties of Formulas A-P

Mechanical and optical properties were measured for the films of the formulas that were fluid enough to allow spreading (Table 4). Formulas B, D, F, H, I, J, and M were too thick and had a balm like texture which would require high shear to apply and hence could not form a uniform film on a substrate.

a) INTERNAL STRESS: For each sample with a texture conducive to spreading, a film was prepared on a flexible nitrile substrate by applying 10 mg/cm² of the formula. The sample were allowed to dry at room temperature for 24 hours. The decrease in the area of the substrate due to the internal stress of the film was measured and assigned a score from 1-5, ranging between (1) none/negligible, (2) low, (3) mild, (4) moderate and (5) high.

b) FILM THICKNESS: For each sample with a texture conducive to spreading, a film was prepared on a flexible substrate of polyurethane coated textile, using the 8 mL path of a drawdown bar. The samples were allowed to dry at room temperature for 24 hours. The thickness of the substrate with and without the film was measured using digital calipers, and the film thickness was calculated using the following equation Film Thickness (d) = Thickness of substrate with film (df) - Thickness od substrate (d0)

The calculated film thickness was then assigned a score from 1-5, ranging between (1) none/negligible, (2) low, (3) mild, (4) moderate and (5) high.

c) OPTICAL PROPERTIES: For each sample with a texture conducive to spreading, a film was prepared on a transparent plastic sheet, using the 8 mL path of a drawdown bar. The samples were allowed to dry at room temperature for 24 hours. Transparency and haze were measured using a BYK Hazeguard apparatus and gloss at 60° was measured using a BYK gloss meter.

TABLE 4

Film Properties

| FLA# | INTERNAL STRESS SCORE | FILM THICKNESS SCORE | TRANSPARENCY | HAZE | GLOSS AT 60° |
|---|---|---|---|---|---|
| A | 4 | 2 | 93.3 | 88.2 | 14.8 |
| C | 2 | 2 | 87.8 | 68.9 | 16.3 |
| E | 1 | 3 | 91.8 | 47.7 | 22.9 |
| G | 5 | 2 | 93.0 | 72.0 | 63.5 |
| K | 5 | 4 | 92.0 | 77.5 | 2.9 |
| L | 2 | 4 | 92.1 | 71.5 | 15.8 |
| N | 3 | 5 | 91.6 | 72.0 | 19.3 |
| O | 3 | 5 | 91.9 | 51.8 | 21.1 |
| P | 1 | 5 | 91.9 | 49.4 | 25.0 |

*Scale: (1) none/negligible, (2) low, (3) mild, (4) moderate and (5) high.

### Example 4 Formulas With Additional Film Formers

Compositions T-V were prepared using the weight percentages of the materials listed in Table 5 using the following process.
1. Glycerin was incorporated into Dispersion A by mixing the two materials at 2750 rpm for 5 mins in a speedmixer.
2. Additional film former, polyhydroxystearic acid and isododecane were added to the dispersion from step 1 and mixed at 2750 rpm for 3 min.
3. Finally, Silica silylate was added and the mixture was further mixed at 2750 rpm for 3 min.
4. The formula was allowed to rest for 18-24 hours before performing any tests.

TABLE 5

Composition of formulas T-V by weight percent of each raw material

| FLA # | OTHER FILM FORMER | GLYCERIN | DISPERSION A | POLYHYDROXY STEARIC ACID | ISODODECANE | SILICA SILYLATE |
|---|---|---|---|---|---|---|
| T | ACRYLATES/ISOBORNYL ACRYLATE COPOLYMER | 19.25 | 53.45 | 1.99 | 23.31 | 2 |
| U | ACRYLIC ACID/ ISOBUTYL ACRYLATE/ ISOBORNYL ACRYLATE COPOLYMER | 19.25 | 53.45 | 1.99 | 23.31 | 2 |
| V | HYDROGENATED STYRENE/ ISOBUTENE COPOLYMER | 19.25 | 53.45 | 1.99 | 23.31 | 2 |

a) FILM PROPERTIES: Film properties were analyzed using the methods explained in Example 2 and are presented in table 6.

TABLE 6

| FLA # | INTERNAL STRESS SCORE | FILM THICKNESS SCORE | TRANSPARENCY | HAZE | GLOSS AT 60° |
|---|---|---|---|---|---|
| T | 2 | 5 | 91.80 | 59.00 | 19.30 |
| U | 1 | 5 | 91.90 | 57.00 | 19.30 |
| V | 1 | 5 | 91.80 | 67.30 | 16.40 |

### Example 5 Formulas With Pigments

Pigmented compositions Q-S (Table 7) were prepared using the base architecture of composition O, with 5% of the volatile oil (isododecane) being replaced by pigments. The formulations were prepared using the following process:
5. Glycerin was incorporated into Dispersion A by mixing the two materials at 2750 rpm for 5 mins in a speedmixer.
6. Polyhydroxystearic acid and isododecane were added to the dispersion from step 1 and mixed at 2750 rpm for 3 min.
7. Silica silylate was added and the mixture was further mixed at 2750 rpm for 3 min.
8. Finally, pigments were added to the white cream obtained after step 4, and mixed at 2750 rpm for 3 min.
9. The formula was allowed to rest for 18-24 hours before performing any tests.

TABLE 7

Composition of formulas Q-S by weight percent of each raw material

| FLA # | PIGMENT TYPE | GLYCERIN | DISPERSION A | POLYHYDROXY STEARIC ACID | ISODODEC ANE | SILICA SILYLATE | PIGMENTS |
|---|---|---|---|---|---|---|---|
| Q | Hydrophilic-Algin Treated | 19.25 | 53.45 | 1.99 | 18.53 | 1.78 | 5 |
| R | Hydrophilic-Silica Shell | 19.25 | 53.45 | 1.99 | 18.53 | 1.78 | 5 |
| S | Hydrophobic-Alumina Flakes | 19.25 | 53.45 | 1.99 | 18.53 | 1.78 | 5 |

FILM ANALYSIS: The internal stress and thickness of the films of pigmented formulas was measured and scored using the process explained in Example 2 and presented in Table 8.

TABLE 8

| FLA# | INTERNAL STRESS SCORE | FILM THICKNESS SCORE |
|---|---|---|
| Q | 4 | 2 |
| R | 3 | 2 |
| S | 1 | 3 |

*Scale: (1) none/negligible, (2) low, (3) mild, (4) moderate and (5) high.

The mechanical performance, as indicated by the internal stress score, is higher for the formulas with hydrophilic pigments. Hydrophobic pigments take residence in the continuous oil phase which is the film forming phase of the formula, thus causing defects in the formulas. Although, no visual cracking of the film is observed upon stretching, the film with hydrophobic film formers is weakened due to the disruption of the network by pigments.

### Example 6 Emulsion

An emulsion based formulation W was prepared using the weight percentages of the raw materials (RM) listed in Table 9:

TABLE 9

Composition of formula W by weight percent of each raw material

| RM | % WT. | ROLE |
|---|---|---|
| MAGNESIUM SULFATE | 0.25 | Charge |
| DE-IONIZED WATER | 16.13 | Solvent |
| PHENOXYETHANOL | 0.7 | Preservative |
| CAPRYLYL GLYCOL | 0.5 | Preservative |
| DENATURED ALCOHOL | 2 | Preservative |
| LAURYL PEG-9 POLYDIMETHYLSILOXYETHYL DIMETHICONE | 1.49 | Surfactant |
| DIMETHICONE (and) PEG/PPG-18/18 DIMETHICONE | 1.99 | Surfactant |
| DISPERSION A | 53.4 | Film Former |
| GLYCERIN | 7.8 | Volume/Elasticity enhancer |
| POLYHYDROXYSTEARIC ACID | 1.5 | Dispersant |
| ISODODECANE | 12.74 | Solvent |
| SILICA SILYLATE | 1.5 | Filler |

The resulting formulation was a cream which forms a film with transparency at 91.7% and Haze at 77.7%, as analyzed using the method described in Example 2(c). These numbers indicate a good bluring and soft focus cosmetics effect.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the instant disclosure concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the instant disclosure to the form disclosed herein. Also, it is intended to the appended claims be construed to include alternative embodiments.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the skin tightening compositions of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

As used herein, the terms “comprising,” “having,” and “including” are used in their open, non-limiting sense.

The terms “a,” “an,” and “the” are understood to encompass the plural as well as the singular. Thus, the term “a combination thereof” also relates to “combinations thereof.” Throughout the disclosure, the term “a combination thereof” is used, following a list of elements as shown in the following example where letters A-F represent the elements: “one or more elements selected from the group consisting of A, B, C, D, E, F, and a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from the group consisting of A, B, C, D, E, F, and a combination of any two or more of A, B, C, D, E, and F.”

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/- 5% of the indicated number.

The term “treat” (and its grammatical variations) as used herein refers to the application of the compositions of the present disclosure onto the surface of keratinous substrates such as skin, in particular, the skin of the head, face, and neck.

The term “substantially free” or “essentially free” as used herein means that there is less than about 2% by weight of a specific material added to a composition, based on the total weight of the compositions. Nonetheless, the compositions may include less than about 1 wt.%, less than about 0.5 wt.%, less than about 0.1 wt.%, or none of the specified material. All of the components set forth herein may be optionally included or excluded from the compositions/method/kits. When excluded, the compositions/methods/kits may be free or essentially free of the component. For example, a particular composition may be free or essentially free of alkoxylated compounds, for example, ethoxylated thickening agents and/or ethoxylated surfactants. Likewise, a particular composition may be free or essentially free of sulfates, such as sulfate surfactants.

The term “film-forming polymer”, “film former”, “film forming agent” are used interchangeably.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%- 11% and “about 2%” means 1.8%- 2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

The terms “weight percent” and “wt%” may be used interchangeably and mean percent by weight, based on the total weight of a composition, article or material, except as may be specified with respect to, for example, a phase, or a system that is a component of a composition, article or material. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

What is claimed is:

1. A skin tightening composition comprising:
A. a dispersion (A), which comprises:
i) at least two particle (s) of different chemical structures consisting of polymer (s) obtained by polymerization of monomers chosen from ethylenic monomers of:
a₁) (C₁-C₄)alkyl (C₁-C₄)(alkyl)acrylate in which the (C₁-C₄) alkyl group (s) are optionally substituted by one or more group (s) chosen from hydroxy, and (di) (C₁ -C₄) (alkyl) amino,
a₂) poly[oxy(C₁-C₄)alkylene] (C₁-C₄)(alkyl)acrylate,
a₃) ethylenic monomers comprising one or more groups selected from carboxy, anhydride, phosphoric acid, and sulfonic acid;
it being understood that each particle results from the polymerization of one or more ethylenic monomer(s) a3); and
ii) one or more polymeric stabilizing agent (s) chosen from:
d) ethylenic polymers of (C1-C6) (alkyl) acrylate of (C3-C12) cycloalkyl monomers, preferably ethylenic homopolymers of (C1-C6) (alkyl) acrylate of (C3-C12) cycloalkyl, more preferably ethylenic homopolymers of (C3-C12) cycloalkyl (meth) acrylate; and
e) copolymers of ethylenic monomers of:
e1) (C1-C6) (C3-C12) cycloalkyl (alkyl) acrylate, and
e2) (C₁-C₄)alkyl (C₁-C₄)(alkyl)acrylate, preferably copolymers of (C3-C12) cycloalkyl (meth) acrylate and (C1- C4) alkyl (meth) acrylate; and
iii) one or more liquid fatty substances at 20° C. and 1 atmosphere, preferably volatile hydrocarbon (s); and iv) one or more polyol (s) liquid (s) at 20° C. and 1 atmosphere; and v) possibly water; and vi) optionally one or more cosmetic active (s) other than iii) and iv), chosen from f) dyes, g) pigments; h) active agents for caring for keratin materials, in particular the skin, in particular antioxidants and moisturizers other than iv), i) UV filters (A) and / or (B), or j) mixtures of f) to i).
B. one ore more polyols, in addition to the one or more polyol(s) from A)iv), which may be the same or different than the one from A)iv);
C. one or more volatile hydrocarbon, in addition to the one or more volatile hydrocarbon(s) from A)iii), which may be the same or different than the one from A)iii);
D. one or more fillers; and
wherein the weight ratio of the total amount of i) of the dispersion A to the total amount of the amount of polyols B is between around 1:1 to 4:1;
wherein the weight ratio of the total amount of i) of the dispersion A to the total amount of filler D (dispersion A: filler) is between around 1:1 to 10:1;
wherein all percentages by weight are based on the total weight of the skin tightening composition.

2. The skin tightening composition of claim 1, wherein the dispersion (A) comprises at least one particle i1) consisting of copolymer derived from monomers a₁) and / or a₂) and a₃) and at least one particle i2) of different chemical composition of i1) chosen from a copolymer derived from monomers a₁) and / or a₂) and a₃), a copolymer derived from monomers a₃), a homopolymer derived from monomers a₃).

3. The skin tightening composition of claim 1, wherein the dispersion (A) comprises at least two particle (s) of different chemical structures i) containing the particles A1 consisting of copolymers of ethylenic monomers:
a₁) (C₁-C₄)alkyl (C₁-C₄)(alkyl)acrylate in which the (C₁-C₄) alkyl group (s) are optionally substituted by one or more group (s) chosen from hydroxy, and (di) (C₁-C₄) (alkyl) amino; and/or
a₂) poly[oxy(C₁-C₄)alkylene] (C₁-C₄)(alkyl)acrylate, and
a₃) ethylenic monomers comprising one or more carboxy, anhydride, phosphoric acid, sulfonic acid, preferably consisting of copolymers of ethylenic monomers a₁) and a₃), or a₂) and a₃), more preferably a₁) and a₃).

4. The skin tightening composition of claim 1, wherein the dispersion (A) comprises at least two particle(s) of different chemical structures i) containing particles A′1 consisting of copolymers of ethylenic monomers a′₁) (C₁-C₄) (alkyl) (C₁-C₄) alkyl acrylate and a₃) ethylenic monomers comprising one or more carboxy, anhydride, phosphoric acid, sulfonic acid, and / or groups containing particles A′2 consisting of copolymers of ethylenic monomers a″1) (C1-C4) (C1-C4) alkyl (alkyl) acrylate substituted with one or more groups chosen from hydroxy, (di) ((C1- C4) alkyl) amino, preferably substituted with a hydroxy group such as 2-hydroxyethylacrylate (HEA) and a₃) ethylenic monomers comprising one or more carboxy, anhydride, phosphoric acid, sulfonic acid groups; preferably, the particles i) of the dispersion (A) and more preferably the particles i) of the dispersion (A) are a mixture of particles A′1 and A′2, in particular in a mass ratio (mass of particles A′1 / mass of particles A′2) between 0.3 and 3, more particularly between 0.5 and 2.8, even more preferably between 0.6 and 2.

5. The skin tightening composition of claim 1, wherein the dispersion (A) comprises the polymer (s) constituting the particles i) is (are) chosen from ethylenic copolymers resulting from the polymerization of monomer of formula (I) with ethylenic monomers comprising one or more carboxy, anhydride, phosphoric acid, sulfonic acid a3): 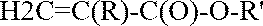
Formula (I) in which:
R representing a hydrogen atom or a (C₁-C₄) alkyl group such as methyl, and
R ‘representing a (C1-C4) alkyl group such as methyl or ethyl, preferably the compound (s) of formula (I) is (are) chosen from C₁-C₄ alkyl (meth) acrylates such as methyl (meth) acrylate, (meth) acrylate ethyl, n-propyl (meth) acrylate, isopropyl (meth) acrylate, n-butyl (meth) acrylate, or tert-butyl (meth) acrylate; more preferably chosen from methyl (meth) acrylate, ethyl (meth) acrylate, even more preferably the compound (s) of formula (I) represents (s) a C₁-C₄ alkyl acrylate such as methyl acrylate.

6. The skin tightening composition of claim 1, wherein the dispersion A is present from at least about 5 wt.% based on the total weight of the skin tightening composition.

7. The skin tightening composition of claim 1, wherein the one or more polyols is selected from glycerine, propandiol, butylene glycol, propylene glycol and mixture thereof.

8. The skin tightening composition of claim 1, wherein the one or more polyols is present from about 1 to about 50 wt.% based on the total weight of the skin tightening composition.

9. The skin tightening composition of claim 1, wherein the one or more volatile hydrocarbon is chosen from isoparaffin, isohexadecane, isododecane, isodecane, undecane, tridecane, dodecane, isohexyl, isodecyl, neopentanoate, and a mixture thereof.

10. The skin tightening composition of claim 1, wherein the one or more volatile hydrocarbon is present from about 10 to about 85 wt.% based on the total weight of the skin tightening composition.

11. The skin tightening composition of claim 1, wherein the one or more fillers is chosen from silica silylate, fumed silica, zeolite, natural clay, synthetic clay, kaolin, hectorite, organically modified hectorite, pentaerythrityl tetraisostearate (and) disteardimonium hectorite (and) propylene carbonate, an activated clay, disteardimonium hectorite, stearalkonium hectorite, quaternium-18 bentonite, quaternium-18 hectorite, benzalkonium bentonite, and a mixture thereof.

12. The skin tightening composition of claim 1, wherein the one or more fillers is present from about 1 to about 20 wt.% based on the total weight of the skin tightening composition.

13. The skin tightening composition of claim 1, wherein the weight ratio of the total amount of i) of the dispersion A to the total amount of filler (film forming polymers : filler) is between around 1:1 to 7:1.

14. The skin tightening composition of claim 1, wherein the weight ratio of the total amount of i) of dispersion A to the total amount of the amount of polyols B is between around 1:1 to 4:1;.

15. The skin tightening composition of claim 1, further comprising one or more pigments, in addition to the one or more pigments from A)vi)g, which may be the same of different than the one from A)vi)g.

16. The skin tightening composition of claim 1, further comprising one or more surfactant.

17. A method for improving the appearance of skin comprising applying a skin tightening composition of claim 1 to the skin.

18. A skin tightening composition comprising:
a. at least 5 wt.% of a dispersion of a film forming hydrophobic polymer encapsulating a hydrogel in a volatile organic non-polar solvent;
b. from about 1 to about 50 wt.% of one ore more organic polyols;
c. from about 10 to about 85 wt.% of one or more volatile hydrocarbons;
d. from about 1 to about 20 wt.% of one or more fillers;
e. optionally one or more pigments; and
wherein the weight ratio of the total amount of the film forming hydrophobic polymer to the total amount of filler (film forming polymers: filler) is between around 1:1 to 10:1;
wherein the weight ratio of the total amount of the film forming hydrophobic polymer to the total amount of the total amount of polyols is between around 1:1 to 4:1 ;
wherein all percentages by weight are based on the total weight of the skin tightening composition.

19. A skin tightening composition comprising:
A. at least 5 wt.% of a dispersion of acrylates based polymer encapsulating a hydrogel in a volatile organic non-polar solvent;
B. from about 1 to about 50 wt.% of glycerine;
C. from about 30 to about 85 wt.% of one or more volatile hydrocarbon;
wherein the weight ratio of the total amount of the dispersion of acrylates based polymer encapsulating a hydrogel in a volatile organic non-polar solvent to the total amount of filler (film forming polymers : filler) is between around 1:1 to 8:1;
D. from about 1 to about 20 wt.% of one or more fillers;
wherein the weight ratio of the total amount of the a dispersion of acrylates based polymer encapsulating a hydrogel in a volatile organic non-polar solvent to to the total amount of the total amount of polyols is between around 1:1 to 4:1 ;
wherein all percentages by weight are based on the total weight of the skin tightening composition.
